SEQUENCE LISTING (49 sequences)

SEQ ID NO: 1 - DNA, 321 bp, Mus musculus
  source 1..321: mol_type = genomic DNA
gacatccagatgacacaatcttcatcctccttttctgtatctctaggagacagagtcaccattacttgcaaggcaagtgaggacatatataatcgattagcctggtatcagcagaaaccaggaaatgctcctaggctcttaatatcaggtgtaagcagtttggaagctggaattccttcaagattcagtggcagtggatctggaaaggattacactctcagcatttccagtcttcagactgaagatgttgctgcttattactgtcaacagtattggagttctccgtacacgttcggaggggggaccaaactggaaataaaa

SEQ ID NO: 2 - protein, 107 aa, Mus musculus
DIQMTQSSSSFSVSLGDRVTITCKASEDIYNRLAWYQQKPGNAPRLLISGVSSLEAGIPSRFSGSGSGKDYTLSISSLQTEDVAAYYCQQYWSSPYTFGGGTKLEIK

SEQ ID NO: 3 - DNA, 363 bp, Mus musculus
  source 1..363: mol_type = genomic DNA
gaagtgaagctggtggagtctgggggaggcttagtgcagcctggagggtccctgaaactctcctgtgcagcctctggattcactttcaggaccgcttttatgtcttggattcgccaaactccagagaagaggctggaattggtcgcagaaattagtgatagtggtggcagcacctacttttcagacactgtaaagggccgatttaccatctccagagacaatgccaagaacagtctgtacctgcaaatgagtagtctgaagtctgaggacacggccatgtattactgtgcaagacggggacagctcggactacggggattctttgactgctggggccaaggcaccactctcacagtctcctca

SEQ ID NO: 4 - protein, 121 aa, Mus musculus
EVKLVESGGGLVQPGGSLKLSCAASGFTFRTAFMSWIRQTPEKRLELVAEISDSGGSTYFSDTVKGRFTISRDNAKNSLYLQMSSLKSEDTAMYYCARRGQLGLRGFFDCWGQGTTLTVSS

SEQ ID NO: 5 - protein, 8 aa, Mus musculus
EDIYNRLA

SEQ ID NO: 6 - protein, 7 aa, Mus musculus
GVSSLEA

SEQ ID NO: 7 - protein, 9 aa, Mus musculus
QQYWSSPYT

SEQ ID NO: 8 - protein, 10 aa, Mus musculus
GFTFRTAFMS

SEQ ID NO: 9 - protein, 17 aa, Mus musculus
EISDSGGSTYFSDTVKG

SEQ ID NO: 10 - protein, 12 aa, Mus musculus
RGQLGLRGFFDC

SEQ ID NO: 11 - DNA, 318 bp, Mus musculus
  source 1..318: mol_type = genomic DNA
gatatccagatgacacagactacatcctccctgtctgcctctctgggagacagagtcaccatcgattgcagggcgagtcaggacatcaacaattatttaaactggtatcaacagaaaccagatggaactgttaaactcctgatccactacacatcaagattacactcaggagtcccatcaaggttcagaggcagtgggtctggaacagatttttctctcaccattaccaacctggaacaagaagatgttgccacttacttttgtcaacagggtaatacgatgtggacgttcggtggaggcaccaagttggatatcaaa

SEQ ID NO: 12 - protein, 106 aa, Mus musculus
DIQMTQTTSSLSASLGDRVTIDCRASQDINNYLNWYQQKPDGTVKLLIHYTSRLHSGVPSRFRGSGSGTDFSLTITNLEQEDVATYFCQQGNTMWTFGGGTKLDIK

SEQ ID NO: 13 - DNA, 354 bp, Mus musculus
  source 1..354: mol_type = genomic DNA
gaagtgaaactggtggagtctgggggaggcttagtggagcctggaacgtctctgaaactctcctgtgcagcctctgaattcactttcagtaattatggaatgtcttgggttcgccagacttcagacaagaggctggagtgggtcgcatccattagtagtggtaatggggtttattattcagacaatgtaaagggccgattcaccatctccagagagaatgccaagaacaccctgtatttgcaaatgagtagtctgaagtctgaagacacggccttatattactgtgcaagagttgatggtagttatgtatatttcgatgtctggggctcagggaccacggtcaccgtctcctca

SEQ ID NO: 14 - protein, 118 aa, Mus musculus
EVKLVESGGGLVEPGTSLKLSCAASEFTFSNYGMSWVRQTSDKRLEWVASISSGNGVYYSDNVKGRFTISRENAKNTLYLQMSSLKSEDTALYYCARVDGSYVYFDVWGSGTTVTVSS

SEQ ID NO: 15 - protein, 8 aa, Mus musculus
QDINNYLN

SEQ ID NO: 16 - protein, 7 aa, Mus musculus
YTSRLHS

SEQ ID NO: 17 - protein, 8 aa, Mus musculus
QQGNTMWT

SEQ ID NO: 18 - protein, 10 aa, Mus musculus
EFTFSNYGMS

SEQ ID NO: 19 - protein, 16 aa, Mus musculus
SISSGNGVYYSDNVKG

SEQ ID NO: 20 - protein, 10 aa, Mus musculus
VDGSYVYFDV

SEQ ID NO: 21 - protein, 107 aa, Mus musculus
DIQMTQSPSSLSASVGDRVTITCKASEDIYNRLAWYQQKPGKAPKLLLYGVSSLEAGVPSRFSGSGSGTDYTLTISSLQPEDFATYYCQQYWSSPYTFGQGTKLEIK

SEQ ID NO: 22 - protein, 107 aa, Mus musculus
DIQMTQSPSSLSASVGDRVTITCKASEDIYNRLAWYQQKPGKAPKLLISGVSSLEAGIPSRFSGSGSGTDYTLTISSLQPEDFAAYYCQQYWSSPYTFGQGTKLEIK

SEQ ID NO: 23 - protein, 107 aa, Mus musculus
DIQMTQSPSSLSASVGDRVTITCKASEDIYNRLAWYQQKPGNAPKLLISGVSSLEAGIPSRFSGSGSGTDYTLTISSLQPEDVAAYYCQQYWSSPYTFGQGTKLEIK

SEQ ID NO: 24 - protein, 107 aa, Mus musculus
DIQMTQSPSSLSASVGDRVTITCKASEDIYNRLAWYQQKPGNAPKLLISGVSSLEAGIPSRFSGSGSGKDYTLTISSLQPEDVAAYYCQQYWSSPYTFGQGTKLEIK

SEQ ID NO: 25 - protein, 121 aa, Mus musculus
EVKLVESGGGLVQPGGSLKLSCAASGFTFRTAFMSWIRQTPEKRLELVAEISDSGGSTYFSDTVKGRFTISRDNAKNSLYLQMSSLKSEDTAMYYCARRGQLGLRGFFDSWGQGTTLTVSS

SEQ ID NO: 26 - protein, 121 aa, Mus musculus
EVQLVESGGGLVQPGGSLRLSCAASGFTFRTAFMSWVRQAPGKGLEWVSEISDSGGSTYFSDTVKGRFTISRDNAKNSLYLQMNSLRAEDTAVYYCARRGQLGLRGFFDSWGQGTLVTVSS

SEQ ID NO: 27 - protein, 121 aa, Mus musculus
EVQLVESGGGLVQPGGSLRLSCAASGFTFRTAFMSWIRQAPGKGLELVAEISDSGGSTYFSDTVKGRFTISRDNAKNSLYLQMNSLRAEDTAVYYCARRGQLGLRGFFDSWGQGTLVTVSS

SEQ ID NO: 28 - protein, 121 aa, Mus musculus
EVQLVESGGGLVQPGGSLRLSCAASGFTFRTAFMSWIRQAPEKRLELVAEISDSGGSTYFSDTVKGRFTISRDNAKNSLYLQMNSLRAEDTAMYYCARRGQLGLRGFFDSWGQGTLVTVSS

SEQ ID NO: 29 - DNA, 22 bp, synthetic construct
  source 1..22: mol_type = other DNA
tccagaacacgatgaggcagac

SEQ ID NO: 30 - DNA, 22 bp, synthetic construct
  source 1..22: mol_type = other DNA
gtagacgacaccagcaacaagg

SEQ ID NO: 31 - DNA, 20 bp, synthetic construct
  source 1..20: mol_type = other DNA
ggcgacctggaagtccaact

SEQ ID NO: 32 - DNA, 20 bp, synthetic construct
  source 1..20: mol_type = other DNA
ccatcagcaccacagccttc

SEQ ID NO: 33 - DNA, 26 bp, synthetic construct
  source 1..26: mol_type = other DNA
ctgctcactggatggtgggaagatgg

SEQ ID NO: 34 - DNA, 20 bp, synthetic construct
  source 1..20: mol_type = other DNA
agctgggaaggtgtgcacac

SEQ ID NO: 35 - DNA, 33 bp, synthetic construct
  source 1..33: mol_type = other DNA
cggggattctttgactcctggggccaaggcacc

SEQ ID NO: 36 - DNA, 33 bp, synthetic construct
  source 1..33: mol_type = other DNA
ggtgccttggccccaggagtcaaagaatccccg

SEQ ID NO: 37 - protein, 12 aa, synthetic construct
RGQLGLRGFFDS

SEQ ID NO: 38 - DNA, 37 bp, synthetic construct
  source 1..37: mol_type = other DNA
agcactagtgccgccaccatgaagtttccttctcaac

SEQ ID NO: 39 - DNA, 35 bp, synthetic construct
  source 1..35: mol_type = other DNA
gggcgccgctacagtccgttttatttccagtttgg

SEQ ID NO: 40 - DNA, 35 bp, synthetic construct
  source 1..35: mol_type = other DNA
agctctagagccgccaccatgaatttcgggctcag

SEQ ID NO: 41 - DNA, 32 bp, synthetic construct
  source 1..32: mol_type = other DNA
ggtgctagctgaggagactgtgagagtggtgc

SEQ ID NO: 42 - DNA, 37 bp, synthetic construct
  source 1..37: mol_type = other DNA
agaactagtgccgccaccatgtcctctgctcagttcc

SEQ ID NO: 43 - DNA, 37 bp, synthetic construct
  source 1..37: mol_type = other DNA
ctgggcgccgctacagtccgtttgatatccaacttgg

SEQ ID NO: 44 - DNA, 36 bp, synthetic construct
  source 1..36: mol_type = other DNA
agctctagagccgccaccatgaacttcgggctcagc

SEQ ID NO: 45 - DNA, 36 bp, synthetic construct
  source 1..36: mol_type = other DNA
gtgctagctgaggagacggtgaccgtggtccctgag

SEQ ID NO: 46 - protein, 95 aa, Homo sapiens
DIQMTQSPSSLSASVGDRVTITCRASQGISNSLAWYQQKPGKAPKLLLYAASRLESGVPSRFSGSGSGTDYTLTISSLQPEDFATYYCQQYYSTP

SEQ ID NO: 47 - protein, 98 aa, Homo sapiens
EVQLVESGGGLVQPGGSLRLSCAASGFTFSSYEMNWVRQAPGKGLEWVSYISSSGSTIYYADSVKGRFTISRDNAKNSLYLQMNSLRAEDTAVYYCAR

SEQ ID NO: 48 - protein, 12 aa, Homo sapiens
YTFGQGTKLEIK

SEQ ID NO: 49 - protein, 13 aa, Homo sapiens
DSWGQGTLVTVSS

(19) United States
(12) Patent Application Publication
(10) Pub. No.: US 2026/0285974 A1
(43) Pub. Date: Sep. 24, 2026

(54) Anti human CD166 antibodies and methods of use therefor

(71) Applicant: MedAbome, Inc., Fremont, CA (US)

(72) Inventors: Mason Lu, Fremont, CA (US); Qinhong Ma, Fremont, CA (US); Mary Q. Xu, Houston, TX (US); Daizong Li, Clayton, MO (US); Ke-Wei Zhao, El Monte, CA (US)

(21) Appl. No.: 19/084,767

(22) Filed: Mar. 20, 2025

(51) Int. Cl. C07K 16/28; A61K 39/00; A61K 47/68

(52) U.S. Cl. CPC C07K 16/2803; A61K 47/68031; A61K 2039/505; C07K 2317/24; C07K 2317/565; C07K 2317/567; C07K 2317/569; C07K 2317/76; C07K 2317/77; C07K 2317/92

(57) ABSTRACT

Disclosed are two monoclonal antibodies, MAb52-29.1 and MAb52-25.1 mAbs, along with their fragments and derivatives, that recognize two conformational epitopes of human CD166 (ALCAM, 4F2), which are highly expressed on malignant tumors but weakly expressed or absent in normal human cells and tissues. This specificity enhances their potential as therapeutic agents while minimizing off-target effects. The anti-CD166 mAbs and their fragments and derivatives can be conjugated to cytotoxic drug moieties to form antibody-drug conjugates (ADCs), effectively inhibiting tumor growth in animal models and demonstrating potential for clinical applications.

### SEQUENCE LISTING

The instant application contains a Sequence Listing which has been submitted electronically in XML format and is hereby incorporated by reference in its entirety. Said XML copy, is 46,302 bytes in size, was created on 2025 May 16, and is named 084,767.xml.

### FIELD OF THE INVENTION

The present invention relates to two monoclonal antibodies, MAb52-29.1 and MAb52-25.1 mAbs, and their derivatives that bind specifically with high affinities to distinct conformational epitopes of human CD166. These epitopes are overexpressed in a variety of cancers, but absent or expressed weakly in normal tissues.

### BACKGROUND

CD166 (cluster of differentiation 166), also known as Activated Leukocyte Cell Adhesion Molecule (ALCAM), is a type I transmembrane glycoprotein belonging to the immunoglobulin (Ig) superfamily. It is encoded by the ALCAM gene located on chromosome 3q13.11 in humans (Bowen et al., 1995). The protein consists of 583 amino acids and has a molecular weight of approximately 65~100 kDa as a result of glycosylation. CD166 features an extracellular domain (ECD) comprising five Ig-like domains that mediate cell-cell adhesion through homophilic interaction between two CD166 proteins, or through heterophilic interaction between CD166 and CD6 on adjacent cells. The cytoplasmic domain of CD166 interacts with cytoskeletal proteins like actin via adaptor molecules, facilitating signal transduction and cellular responses. CD166 is ubiquitously expressed in almost all cell types and is primarily recognized as a cell adhesion molecule, playing a pivotal role in mediating cell-cell interactions. Its key physiological activities are very diverse, include stabilizing tissue architecture, and facilitating cellular communication; T-cell activation and trafficking; regulating cell proliferation, migration, and survival; and enhancing “stemness” of stem and progenitor cells (van Kempen et al., 2001; Hassan et al., 2004; Cayrol et al., 2007; Chitteti et al., 2014; von Lersner et al., 2019; Ferragut et al., 2021).

Altered ALCAM gene or CD166 protein expression has been associated with the progression, increased metastatic chances, and poor prognosis in numerous malignancies, including melanoma, prostate cancer, breast cancer (BC), colorectal cancer (CRC), pancreatic ductal adenocarcinoma (PDAC), ovarian cancer (OC), glioblastoma, and others (Ihnen et al., 2008; Darvishi et al., 2020; Hong et al., 2010; Fujiwara et al., 2014; for reviews, see von Lersner et al., 2019; Yang et al., 2021). In recent years, the CD6-CD166 axis has been implicated in the pathogenesis of multiple autoimmune diseases, such as multiple sclerosis (MS), lupus nephritis (LN), rheumatoid arthritis (RA), inflammatory bowel disease, and Sjögren's syndrome (Gurrea-Rubio and Fox, 2022).

Given its overexpression in cancers and roles in tumorigenesis and progress, CD166 has become an emerging target for therapeutic intervention. Monoclonal antibodies targeting CD166 have been explored to disrupt tumor cell adhesion, inhibit cancer stem cell (CSC) properties, or deliver cytotoxic payloads. For instance, a single-chain antibody (scFv), named scFv173, against CD166 has been proposed to reduce CD166-mediated adhesion in breast cancer, both in vitro and in vivo (Wiiger et al., 2010). Another anti-CD166 scFv was equipped as immunoliposome to deliver toxic drugs to CD166-expressing prostate cancer cells (Roth et al., 2007). Lee et al. (2019) used a bispecific antibody-drug conjugate (ADC) comprising anti-EphA2 and anti-CD166 as a model to show improved internalization and anti-tumor potency compared with the monospecific ADCs. Additionally, CD166-based chimeric antigen receptor T (CAR-T) cell therapy shows cytotoxic effect on CD166-positive osteosarcoma cells in vitro and tumor regression in a mouse model (Wang et al., 2019). These studies demonstrate that CD166 might be a promising biomarker for cancer targeting therapy.

While CD166 targeted therapy has promising anti-tumor potency, its ubiquitous expression in the majority of cells and tissues raises concerns on off-target safety issues. Thus, use of CD166 as a target must specifically recognize cancer cells with no or very weak cross-reaction with normal cells or tissues. Thus, high-affinity anti-CD166 mAbs that selectively bind to tumor-specific conformational epitopes on the CD166 molecule were deemed desirable for development.

### SUMMARY

The present invention relates to the treatment of human malignant tumors expressing CD166 with antibody-drug conjugates (ADCs) wherein the antibodies, MAb52-29.1, MAb52-25.1 mAbs, or fragments or derivatives thereof. These antibodies were produced from hybridomas generated by live-cell immunization (LCI) and live-cell high-throughput screening (LC-HTS) technology, which has a proven advantage in generating mAbs specifically targeting cancer-related conformational epitopes that are rarely found in normal cells or tissues. Both mAbs and their chimeric or humanized derivatives bound to various types of cancer cell lines with high affinities while showing very weak interactions with one or two subpopulations of the cells in human peripheral blood mononuclear cells (PBMCs), bone marrow mononuclear cells (BMMCs), and cord blood mononuclear cells (CB-MNCs). Importantly, both mAbs did not react with normal human tissues, indicating potential tumor specificity. MAb52-29.1 mAb exhibited slightly higher affinity than MAb52-25.1 mAb, with K_{D} of approximately 1.12 nM for binding to the recombinant human CD166 protein.

MAb52-29.1 mAb has amino acid sequence of the variable region of the light chain (VL) shown as SEQ ID NO: 2 below. Three complementarity determining regions (CDRs 1 to 3, left to right, respectively shown as SEQ ID NOs: 5, 6, 7) are highlighted in bold and underlined below.

(SEQ ID NO: 2)
DIQMTQSSSSFSVSLGDRVTITCKASEDIYNRLAWYQQKPGNAPRLLIS
GVSSLEAGIPSRFSGSGSGKDYTLSISSLQTEDVAAYYCQQYWSSPYTF
GGGTKLEIK.

MAb52-29.1 mAb has amino acid sequence of the variable region of the heavy chain (VH) shown as SEQ ID NO: 4 below. Three complementarity determining regions (CDRs 1 to 3, left to right, respectively shown as SEQ ID NOs: 8, 9, 10) are highlighted in bold and underlined below.

(SEQ ID NO: 4)
EVKLVESGGGLVQPGGSLKLSCAASGFTFRTAFMSWIRQTPEKRLELVA
EISDSGGSTYFSDTVKGRFTISRDNAKNSLYLQMSSLKSEDTAMYYCAR
RGQLGLRGFFDCWGQGTTLTVSS.

MAb52-25.1 mAb has amino acid sequence of the variable region of the light chain (VL) shown as SEQ ID NO: 12 below. Three complementarity determining regions (CDRs 1 to 3, left to right, respectively shown as SEQ ID NOs: 15, 16, 17) are highlighted in bold and underlined below.

(SEQ ID NO: 12)
DIQMTQTTSSLSASLGDRVTIDCRASQDINNYLNWYQQKPDGTVKLLIH
YTSRLHSGVPSRFRGSGSGTDFSLTITNLEQEDVATYFCQQGNTMWTFG
GGTKLDIK

MAb52-25.1 mAb has amino acid sequence of the variable region of the heavy chain (VH) shown as SEQ ID NO: 14 below. Three complementarity determining regions (CDRs 1 to 3, left to right, respectively shown as SEQ ID NOs: 18, 19, 20) are highlighted in bold and underlined below.

(SEQ ID NO: 14)
EVKLVESGGGLVEPGTSLKLSCAASEFTFSNYGMSWVRQTSDKRLEWVA
SISSGNGVYYSDNVKGRFTISRENAKNTLYLQMSSLKSEDTALYYCARV
DGSYVYFDVWGSGTTVTVSS

When chimeric antibodies comprising the VL and VH sequences of MAb52-29.1 mAb or MAb52-25.1 mAb were conjugated with a cytotoxic agent to form ADCs, they exhibited potent antiproliferative effects across multiple cancer cell lines in vitro. Furthermore, MAb52-29.1-derived ADC effectively suppressed tumor growth in vivo across various cell line-derived xenograft (CDX) mouse models. An ADC derived from a humanized MAb52-29.1 variant, MAb52-29.1HuAbm2-MMAE, retained inhibitory effect on cancer cell growth, reinforcing its potential as a promising therapeutic agent for cancer treatment.

The designed amino acid sequence of the variable region of the light chain of MAb52-29.1HuAbm2, designated as HuVL2, is shown as SEQ ID NO: 22 below, and its three antigenic determinant regions (CDRs 1 to 3, left to right, respectively shown as SEQ ID NOs: 5, 6, 7) are highlighted in bold and underlined below.

(SEQ ID NO: 22)
DIQMTQSPSSLSASVGDRVTITCKASEDIYNRLAWYQQKPGKAPKLLIS
GVSSLEAGIPSRFSGSGSGTDYTLTISSLQPEDFAAYYCQQYWSSPYTF
GQGTKLEIK

The designed amino acid sequence of the variable region of the heavy chain of MAb52-29.1HuAbm2, designated as HuVH2, is shown as SEQ ID NO: 26 below, and three antigenic determinant regions (CDRs 1 to 3, left to right, respectively shown as SEQ ID NOs: 8, 9, 37) are highlighted in bold and underlined.

(SEQ ID NO: 26)
EVQLVESGGGLVQPGGSLRLSCAASGFTFRTAFMSWVRQAPGKGLEWVS
EISDSGGSTYFSDTVKGRFTISRDNAKNSLYLQMNSLRAEDTAVYYCAR
RGQLGLRGFFDSWGQGTLVTVSS

Additionally, MAb52-29.1 and MAb52-25.1 can be deployed as bispecific, multi-specific, or other fragments or antibody derivatives, or dual-drug ADCs, radionuclide antibody conjugates (RACs), CAR-T/NK/microphage/DC therapies, and proteolysis-targeting chimera (PROTAC), offering innovative solutions for cancer therapies.

MAb52-29.1 and MAb52-25.1 ADCs can also be administrated alone or in combination with other therapeutic approaches, such as immunotherapy, chemotherapy, or cell therapy, for the treatment of cancers, such as breast, lung, colorectal, gastric, pancreatic, or skin cancer, particularly those resistant to standard anti-cancer therapies.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1 to 1A-6 show the surface binding of MAb52-29.1 and MAb52-25.1 mAbs to 32 representative human solid tumor cell lines and three immortalized cell lines, Beas-2B, HUVEC, and HEK239FT, as determined by FACS analyses. The meaning of the abbreviations in the figures is: MFI: median fluorescence intensity; FITC: fluorescein isothiocyanate; [mAb]: concentration of monoclonal antibody; TNBC: triple-negative breast cancer; BC: breast cancer; GC: gastric cancer; CRC: colorectal cancer; HCC: hepatocellular carcinoma; PDAC: pancreatic ductal adenocarcinoma; NSCLC: non-small cell lung cancer; SCLC: small cell lung cancer; CC: cervical cancer.

FIGS. 1B-1 and 1B-2 depict the binding of MAb52-29.1 and MAb52-25.1 mAbs to 12 representative hematologic malignant cell lines. The meaning of the abbreviations in the figures is: AML: acute myeloid leukemia; CML: chronic myeloid leukemia; T-ALL: T cell acute lymphoblastic leukemia; B-ALL: B cell acute lymphoblastic leukemia; NHL-BL: non-Hodgkin lymphoma (Burkitt lymphoma).

FIGS. 2A to 2D show the cell staining results of human peripheral blood mononuclear cells (PBMCs), bone marrow mononuclear cells (BMMCs), cord blood mononuclear cells (CB-MNCs), and cynomolgus monkey PBMCs, stained with MAb52-29.1 or MAb52-25.1 mAb. The subpopulations were categorized based on forward scatter (FSC) versus side scatter (SSC).

FIG. 3A shows the IHC result of formalin-fixed, paraffin-embedded (FFPE) NSCLC section reacted with MAb52-29.1 mAb. Tissues were visualized under a microscope at 10× magnification. Blue staining corresponds to hematoxylin counterstaining, which visualizes cells and nuclei, while positive staining appears in brown.

FIG. 3B displays the IHC result of normal human tissue microarray (TMA) stained with MAb52-29.1 mAb and a table summarizing the staining results along with the corresponding tissue locations on the TMA slide. The TMA slide contains 90 different sections, with every three consecutive spots being the same tissue from three distinct donors. Placenta serves as a positive control.

FIG. 4 shows the results of two independent liquid chromatography with tandem mass spectrometry (LC-MS/MS) analyses of the MAb52-25.1 mAb immunoprecipitation (IP) samples. The tables list the proteins with the highest scores. Peptide coverage of CD166 sequence is highlighted in bold. Accession numbers correspond to UniProtKB/Swiss-Prot IDs. PEP: posterior error probability; PSMs: peptide spectrum matches; MW: molecular weight.

FIG. 5A illustrates dose-dependent bindings of MAb52-29.1 and MAb52-25.1 mAbs to the recombinant human CD166 extracellular domain (ECD) determined by an ELISA assay. Anti-6×His-tag mAb serves as a positive control to confirm proper coating of the antigen. Each well was immobilized with 10 ng of rhCD166-ECD-His. Binding of mAb to the antigens was probed with 1:4,000 diluted Peroxidase AffiniPure™ Goat Anti-Mouse IgG (Fcγ fragment specific). A450 represents absorbance at 450-nm wavelength.

FIG. 5B shows a competitive ELISA assay, where binding between the HRP-conjugated MAb52-29.1 mAb (MAb52-29.1 mAb-HRP) was not inhibited by MAb52-25.1 mAb, suggesting that they recognize different epitopes on CD166.

FIG. 5C shows no binding to the recombinant mouse CD166-ECD, rhCD166-ECD-His, by either MAb52-29.1 mAb or MAb52-25.1 mAb in an ELISA assay. A commercial anti-human CD166 mAb and anti-6×His tag mAb served as the assay controls.

FIG. 6 is a Western blot result showing MAb52-29.1 mAb detected two weak bands in the lane loaded with 1.0 μg rhCD166-ECD-His, but no band in cancer cell lysates under non-reducing condition. Anti-human β-actin mAb-HRP served as a loading control. Numbers indicate the molecular weights in kilodalton (kDa) of Broad Multi Color Pre-Stained Protein Standard (GenScript).

FIG. 7 presents the aligned sensorgram traces depicting the association and dissociation curves for MAb52-29.1 mAb binding to rhCD166-ECD-His, extracted from raw data analyzed by ForteBio Octet® QKe System. Anti-Mouse Fc Capture (AMC) biosensors were immobilized with 100 nM MAb52-29.1 mAb. The rhCD166-ECD-His protein was loaded at concentrations of 200, 100, 50, 25, 12.5, and 6.25 nM by 2-fold serial dilutions to allow association and dissociation for 15 min and 30 min, respectively.

FIG. 8 shows the relative expression levels of the ALCAM gene encoding CD166 in representative cancer cell lines, as determined by qRT-PCR analysis. Data were normalized to RPLP0 mRNA and expressed as fold change relative to healthy human PBMC controls. Values represent the mean±SD of triplicate samples. * P<0.05, ** P<0.01 (Student's t test).

FIG. 9 illustrates the effect of MAb52-29.1 mAb on the invasion of various cancer cell lines, where “ctrl” represents the baseline of isotype control mAb-treated groups. Positive values indicate increased invasion, while negative values indicate decreased invasion. Data are represented as the mean±SD of triplicate samples.

FIGS. 10A to 10H are fluorescent images showing internalization of FITC-labeled MAb52-29.1 mAb or MAb52-25.1 mAb by various human cancer cell lines. Cells were incubated with 1 μg/mL MAb52-29.1 mAb-FITC, MAb52-25.1 mAb-FITC, or an isotype control mAb-FITC for one hour at 4° C. After PBS wash, the cells were incubated at 37° C. with 5% CO₂ for 15 min, 30 min, or 2 hours. The dynamics of antibody binding and internalization of each antibody are shown vertically at four different incubation conditions, with 4° C. as the baseline of antibody binding. The pictures were taken at 40× magnification with the same exposure time. Scale bars indicate 100 μm.

FIG. 11 shows that binding between MAb52-29.1 mAb-HRP and rhCD166-ECD-His was inhibited by the unlabeled MAb52-29.1cAbm2 to a similar extent as the parental murine MAb52-29.1 mAb, as determined by a competitive ELISA assay. The plate was immobilized with 10 ng/well rhCD166-ECD-His. cAbm2: chimeric antibody with double cysteine mutations in each heavy chain constant region (Fc); [Ab]: antibody concentration.

FIG. 12 is the aligned sensorgram traces showing association and dissociation curves for MAb52-29.1cAbm2 binding to rhCD166-ECD-His, extracted from raw data by the ForteBio software. The Octet® QKe System was equipped with Anti-Human Fc Capture (AHC) biosensors to immobilize 100 nM MAb52-29.1cAbm2.

FIG. 13 illustrates the dose-dependent binding of MAb52-29.1cAbm2 to the surface of representative cell lines of various cancer types, as determined by FACS analysis.

FIG. 14 is the pictures of MAb52-29.1cAbm2 and MAb52-25.1cAbm2 with (+) or without (−) MMAE conjugation under both reducing and non-reducing conditions in SDS-PAGE. Each lane was loaded with approximately 2.0 μg protein. Left lane is the Broad Multi Color Pre-Stained Protein Standard (GenScript) with the molecular weights indicated in kDa.

FIGS. 15A to 15E illustrate the inhibitory effects of MAb52-29.1cAbm2-MMAE on the proliferation of various human cell lines, as determined by cell counting assays. The non-cell binding isotype control, isotype ctrl cAbm2-MMAE, served as the proliferation baseline for each cell line. A450: absorbance at 450 nm. Data represent the mean±SD of triplicate samples.

FIGS. 16A to 16F illustrate the antitumor effects of MAb52-29.1cAbm2-MMAE on six cancer cell line-derived xenograft (CDX) mouse models. MAb52-29.1cAbm2-MMAE, isotype control cAbm2-MMAE, or PBS was injected intraperitoneally (i.p.) with the dose as indicated on Day 0. Data represents average tumor volume±SD of five mice per group (n=5). PBS: Dulbecco's phosphate buffer saline; MMAE: monomethyl auristatin E.

FIG. 17A depicts the comparison of the parental murine mAb light chain variable region (MoVL) with the select human germline acceptor IGKV1-NL1*01 and four humanized MAb52-29.1 light chain VLs (HuVL1 to HuVL4). CDRs of MAb52-29.1 mAb are highlighted in bold and underlined. Grey and black bars on top of the sequences are the Kabat and Chothia definitions of CDRs, respectively. Back mutations in frameworks are italicized and highlighted.

FIG. 17B depicts the comparison of the parental murine mAb heavy chain variable region (MoVH) with the select human germline acceptor IGHV3-48*03 and four humanized MAb52-29.1 heavy chains VHs (HuVH1 to HuVH4). CDRs of MAb52-29.1 mAb are highlighted in bold and underlined. Grey and black bars on top of the sequences are the Kabat and Chothia definitions of CDRs, respectively. Back mutations in frameworks are italicized and highlighted. The C102S mutation in CDR-H3 is also highlighted in grey.

FIG. 17C shows different combinations of HuVL and HuVH pairs for expressing five different humanized variants, labeled as MAb52-29.1HuAbm2-1 to -5. The back mutations are listed and their differences are marked in bold. All heavy chains contain the C102S point mutation in CDR-H3.

FIG. 18 shows the binding to rhCD166-ECD-His by the humanized MAb52-29.1 variants in ELISA assays. MAb52-29.1HuAbm2-1 and -2 lost binding capability and MAb52-29.1HuAbm2-3 and -4 had reduced binding affinity, whereas MAb52-29.1HuAbm2-3 retained a similar binding curve as MAb52-29.1cAbm2.

FIG. 19 shows the aligned sensorgram traces depicting the association and dissociation curves and the data table of the binding kinetics between MAb52-29.1HuAbm2 and rhCD166-ECD-His at 2-fold serial dilutions (200, 100, 50, 25, 12.5, and 6.25 nM). The Octet® QKe System was equipped with Anti-Human Fc Capture (AHC) biosensors to immobilize 100 nM MAb52-29.1HuAbm2.

FIG. 20 compares the surface binding of MAb52-29.1cAbm2 and MAb52-29.1HuAbm2 to six representative cancer cell lines, as determined by FACS analysis. Each cell line represents a distinct cancer type. An isotype control chimeric antibody (Isotype ctrl cAbm2) served as the negative control.

FIGS. 21A to 21E are fluorescent images showing internalization of CF488-conjugated MAb52-29.1HuAbm2 by six different human cancer cell lines. Cells were incubated with 1 μg/mL MAb52-29.1HuAbm2-CF488, or an isotype control cAbm2-CF488 for one hour at 4° C. to obtain the baselines of antibody binding for each cell line. Antibody internalization was observed at 15 min, 30 min, and 2 hours after incubation at 37° C. The pictures were taken at 40× magnification with the same exposure time. Scale bars indicate 100 μm.

FIG. 22 shows the SDS-PAGE analysis of MAb52-29.1HuAbm2 with (+) or without (−) MMAE conjugation under both reducing and non-reducing conditions. Each lane was loaded with approximately 2.0 μg protein.

FIG. 23 shows in vitro cell counting assays comparing the efficacies of MMAE-conjugated MAb52-29.1HuAbm2 and MAb52-29.1cAbm2 in inhibiting the proliferation of four representative human cancer cell lines. Isotype control cAbm2-MMAE served as the proliferation baseline for each cell line. Data represent the mean±SD of triplicate samples.

### DETAILED DESCRIPTION

### Definitions

The section headings used herein are for organizational purposes only and are not to be construed as limiting the subject matter described. All patents and applications listed herein are incorporated by reference.

Unless defined otherwise, all technical and scientific terms used herein have the same meaning as is commonly understood by one of ordinary skill in the art. In the event that there is a plurality of definitions for a term herein, those in this section prevail unless stated otherwise.

The term “ADCs” refers to antibody-drug conjugates, wherein a binding agent, including a monoclonal antibody, fragment or derivative is conjugated with a drug. Suitable drugs in an ADC include any substance having biological or detectable activity, for example, therapeutic agents, detectable labels, binding agents, and prodrugs, which are metabolized to an active agent in vivo. Suitable drugs in the conjugates preferably include anti-tumor agents, including Monomethyl auristatin E (MMAE) or another anti-tumor agent, for example, a cytotoxin, including maytansine or a derivative thereof, or a derivative of Auristatin, epothilone or a derivative thereof, paclitaxel or a derivative thereof, or a vinca alkaloid compound; or further including: Combretastatin A-4 phosphate, Combretastatin A-4 and its derivatives, indole-sulfa compounds, vinca alkaloids compounds such as vinblastine, vincristine, vindesine, vinorelbine, vinflunine, vinglycinate, anhydrovinblastine, dolastatins 10 and analogues, halichondrin B and Eribulin, indole-3-oxalyl amides, substituted indol-3-oxalyl amides, podophyllotoxins, 7-diethylamino-3-(2′-benzoxazolyl)-coumarin (DBC), discodermolide, Laulimalide; DNA topoisomerase inhibitors such as camptothecin and its derivatives, mitoxantron; mitoguazone; nitrogen mustard analogues such as Chlorambucil, Chlomaphazine, cyclophosphamide, Estramustine, ifosfamide, Mustine, Nitromin, Melphalan, Novembichin, Phenamet, Phenesterine, Prednimustine, Trofosfamide, Uramustine; nitrosoureas such as Carmustine, streptozotocin, Fotemustine, Lomustine, Nimustine, Ranimustine; antibiotics such as the enediyne antibiotics, Dynemicin, Esperamicin, Neocarzinostatin, Aclacinomycin, Actinomycin, Anthroamycin, Azaserine, Bleomycins, actinomycin C, Carabicin, Idarubicin, Carzinophilin, Carminomycin, Actinomycin D, Daunorubicin, Doxorubicin, 6-diazo-5-oxo-L-norleucine, Adriamycin, Epirubicin, Esorubicin, Idarubicin, Marcellomycin, Mitomycins, Mycophenolic acid, Nogalamycin, Olivomycin, Peplomycin, Bofeimeisu, Puromycin, Adriamycin-Fe, Rodorubicin, Streptonigrin, Streptozocin, Tubercidin, Ubenimex, Zinostatin, Zorubicin; folic acid analogues such as Denopterin, Methotrexate, Pteropterin, Trimetrexate, Edatrexate; Purine analogues such as Fludarabine, 6-mercaptopurine, Thiamiprine, Thioguanine; pyrimidine analogues such as Ancitabine, Gemcitabine, Enoxaparin, Azacitidine, 6-Azauridine, Carmofur, Cytarabine, dideoxyuridine, deoxy-fluorouridine, Fluoruridine; androgens such as Calusterone, Dromostanolone propionate, Epitiostanol, Mepitiostance, Testolactone; anti-adrenal compounds such as Aminoglutethimide, Mitotane, Trilostane; trichothecenes such as T-2 toxin, verracurin A, Roridin A and Anguidine; arizidines such as Benzodopa, Carboquone, Meturedopa and Uredopa; platinum analogs such as Cisplatin, Carboplatin, Oxaliplatin, Miriplatin, Etoposide; anti-androgens such as Flutamide, Nilutamide, Bicalutamide, Leuprolide and Goserelin; protein kinase and proteasome inhibitors.

The binding agent is preferably bound to the anti-tumor agent using a linker, or other binding methods can be used. In some embodiments, linkers may be used in the conjugate bonding which can be composed of flexible residues like glycine and serine (one example being four glycine residues followed by a serine, repeated; or simply repeated glycine residues) so that the adjacent protein domains are free to move relative to one another. If one desires to maintain distance between domains, so they cannot interact, rigid linkers are preferred; one example being (repeating): glutamic acid, three alanine residues, lysine. Another rigid linker is (repeating): any amino acid residue and proline. Linkers can be either non-cleavable (e.g., thioether, SMCC, PEG linkers), or cleavable linkers, such as valine-citrulline (Val-Cit, VC) dipeptide, glutamic acid-valine-citrulline (Glu-Val-Cit, GVC) tripeptide and disulfide linkers. U.S. Pat. No. 9,310,373 “Molecular conjugate” discloses a number of hydrazide thiol linkers, and the making of conjugates with them. U.S. Pat. No. 8,518,891 discloses a linker with an aminoarylmethyl or aminoheteroaryl moiety. Bi-functional linkers have also been described. See U.S. Pat. No. 11,040,084.

The binding agent or antibody in the ADC can be a whole antibody or a fragment thereof. The binding agents of the invention include MAb52-29.1 mAb, MAb52-25.1 mAb, and their derivatives, such as full-length antibodies, antibody fragments, fusion proteins, chimeric antibody receptor (CAR), including single-chain variable fragments (scFv) fused to intracellular signaling domains, e.g., the zeta chain of CD3 (CD3ζ). CAR is preferably expressed in CAR immune effector cells including but not limited to T cells, natural killer (NK) cells, microphages, and dendritic cells (DCs).

The term binding agent(s) includes an antibody (both are used interchangeably in singular or plural form) which is an immunoglobulin molecule capable of binding to a target antigen, such as an antigen on lung cancer cells, through at least one antigen recognition site, located in the variable region of the immunoglobulin molecule. As used herein, the term “antibody” encompasses not only intact (i.e., full-length) monoclonal antibodies like MAb52-29.1 or MAb52-25.1, but also antigen-binding fragments thereof (such as Fab, Fab′, F(ab′)₂, Fv, Fd, rIgG, single chain (scFv) or sc(Fv)₂, mutants thereof, fusion proteins comprising an antibody portion, humanized antibodies (like MAb52-29.1HuAbm2), chimeric antibodies (like MAb52-29.1cAbm2 or MAb52-25.1cAbm2), diabodies, multi-specific antibodies (e.g., bispecific antibodies), single domain antigen binding (SDAB) molecules, a VH or VL domain, or a VHH domain, and any other modified configuration of the immunoglobulin molecule that comprises an antigen recognition site of the required specificity, including glycosylation variants of antibodies, amino acid sequence variants of antibodies, and covalently modified antibodies. Also included are antibody-drug conjugates.

An antibody includes antibodies of any class, such as IgD, IgE, IgG, IgA, or IgM (or a sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgAQ1 and IgA2.

Antibodies described herein can be murine, rat, human, or any other origin (including chimeric or humanized antibodies, including as described in U.S. Pat. No. 7,317,091B2, and including such antibodies generated by affinity maturation). In some examples, the antibody comprises a modified constant region, such as a constant region that is immunologically inert, e.g., does not trigger complement mediated lysis, or does not stimulate antibody-dependent cell mediated cytotoxicity (ADCC).

Humanized antibodies refer to forms of non-human (e.g. murine) antibodies that are specific chimeric immunoglobulins, immunoglobulin chains, or antigen-binding fragments thereof that contain sequences derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody will also optimally comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody. Humanized antibodies may also involve affinity maturation. See Jones et al., Nature, 321:522-525 (1986); Riechmann et al., Nature, 332:323-327 (1988); Verhoeyen et al., Science, 239:1534-1536 (1988).

A chimeric antibody includes a heavy chain constant region and a light chain constant region from a human antibody. See, e.g., Morrison et al. (1984) Proc. Natl. Acad. Sci. USA 81, 6851; Neuberger et al. (1984) Nature 312, 604; and Takeda et al. (1984) Nature 314:452. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region. See U.S. Pat. No. 4,816,567.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the invention. In addition, the term “comprising” is to be interpreted synonymously with the phrases “having at least” or “including at least”. When used in the context of a process, the term “comprising” means that the process includes at least the recited steps but may include additional steps. When used in the context of a compound, composition or device, the term “comprising” means that the compound, composition or device includes at least the recited features or components but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Additionally, the phrase “consisting essentially of” will be understood to include those elements specifically recited and those additional elements that do not materially affect the basic and novel characteristics of the claimed technology. The phrase “consisting of” excludes any element not specified.

### Formulations

Antibodies and Binding Agents and ADCs can be prepared in a formulation for administration to a subject. A lyophilized formulation is preferred, which as a first step, requires preparing a pre-lyophilized formulation. The amount of ADC in the pre-lyophilized formulation is determined taking into account the desired dose volumes, mode(s) of administration, etc. The protein is generally present in solution. For example, the protein may be present in a pH-buffered solution at a pH from about 4-8, and preferably from about 5-7. Exemplary buffers include histidine, phosphate, Tris, citrate, succinate and other organic acids. The buffer concentration can be from about 1 mM to about 20 mM, or from about 3 mM to about 15 mM, depending, for example, on the buffer and the desired isotonicity of the formulation (e.g. of the reconstituted formulation). The preferred buffer is histidine as it can have lyoprotective properties. Succinate is also a useful buffer.

The lyoprotectant is added to the pre-lyophilized formulation. In preferred embodiments, the lyoprotectant is a non-reducing sugar such as sucrose or trehalose. The amount of lyoprotectant in the pre-lyophilized formulation is generally such that, upon reconstitution, the resulting formulation will be isotonic, as preferred, though hypertonic reconstituted formulations may also be suitable. In addition, the amount of lyoprotectant must not be too low such that an unacceptable amount of degradation/aggregation of the protein occurs upon lyophilization.

Where the lyoprotectant is a sugar (such as sucrose or trehalose) and the protein is an antibody, exemplary lyoprotectant concentrations in the pre-lyophilized formulation are from about 10 mM to about 400 mM, and preferably from about 30 mM to about 300 mM, and most preferably from about 50 mM to about 100 mM.

The ratio of protein to lyoprotectant is selected for each protein and lyoprotectant combination. In the case of an antibody as the protein of choice and a sugar (e.g., sucrose or trehalose) as the lyoprotectant for generating an isotonic reconstituted formulation with a high protein concentration, the molar ratio of lyoprotectant to antibody may be from about 100 to about 1500 moles lyoprotectant to 1 mole antibody, and preferably from about 200 to about 1000 moles of lyoprotectant to 1 mole antibody, including from about 200 to about 600 moles of lyoprotectant to 1 mole antibody.

In preferred embodiments, it has been found to be desirable to add a surfactant to the pre-lyophilized formulation. Alternatively, or in addition, the surfactant may be added to the lyophilized formulation and/or the reconstituted formulation. Exemplary surfactants include nonionic surfactants such as polysorbates (e.g. polysorbates 20 or 80); poloxamers (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palnidopropyl-, or isostearamidopropyl-betaine (e.g lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethyl glycol, polypropyl glycol, and copolymers of ethylene and propylene glycol (e.g. Pluronics, PF68 etc). The amount of surfactant added is such that it reduces aggregation of the reconstituted protein and minimizes the formation of particulates after reconstitution. For example, the surfactant may be present in the pre-lyophilized formulation in an amount from about 0.001-0.5%, and preferably from about 0.005-0.05%.

A mixture of the lyoprotectant (such as sucrose or trehalose) and a bulking agent (e.g. mannitol or glycine) may be used in the preparation of the pre-lyophilization formulation. The bulking agent may allow for the production of a uniform lyophilized cake without excessive pockets therein.

Other pharmaceutically acceptable carriers, excipients or stabilizers such as those described in Remington's Pharmaceutical Sciences 16th edition, Osol, A. Ed. (1980) may be included in the pre-lyophilized formulation (and/or the lyophilized formulation and/or the reconstituted formulation) provided that they do not adversely affect the desired characteristics of the formulation. Acceptable carriers, excipients or stabilizers are nontoxic to recipients at the dosages and concentrations employed and include; additional buffering agents; preservatives; co-solvents; antioxidants including ascorbic acid and methionine; chelating agents such as EDTA; metal complexes (e.g. Zn-protein complexes); biodegradable polymers such as polyesters; and/or salt-forming counterions such as sodium. In addition, various adjuvants such as are commonly used in the art may be included. Considerations for the inclusion of various components in pharmaceutical compositions are described, e.g., in Gilman et al. (Eds.) (1990), Goodman and Gilman's: The Pharmacological Basis of Therapeutics, 8th Ed., Pergamon Press, which is incorporated herein by reference in its entirety.

The pharmaceutical compositions and formulations described herein are preferably stable, so as to retain its physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period.

The formulations to be used for in vivo administration must be sterile. This is readily accomplished by filtration through sterile filtration membranes, prior to, or following, lyophilization and reconstitution. Alternatively, sterility of the entire mixture may be accomplished by autoclaving the ingredients, except for protein, at about 120° C. for about 30 minutes.

After the protein, lyoprotectant and other optional components are mixed together, the formulation is lyophilized. Many different freeze-dryers are available for this purpose such as

Hull50® (Hull, USA) or GT20® (Leybold-Heraeus, Germany) freeze-dryers. Freeze-drying is accomplished by freezing the formulation and subsequently subliming ice from the frozen content at a temperature suitable for primary drying. Under this condition, the product temperature is below the eutectic point or the collapse temperature of the formulation.

Typically, the shelf temperature for the primary drying will range from about-30 to 25° C. (provided the product remains frozen during primary drying) at a suitable pressure, ranging typically from about 50 to 250 mTorr. The formulation, size and type of the container holding the sample (e.g., glass vial) and the volume of liquid will mainly dictate the time required for drying, which can range from a few hours to several days (e.g. 40-60 hours). A secondary drying stage may be carried out at about 0-40° C., depending primarily on the type and size of container and the type of protein employed. For example, the shelf temperature throughout the entire water removal phase of lyophilization may be from about 15-30° C. (e.g., about 20° C.). The time and pressure required for secondary drying will be that which produces a suitable lyophilized cake, dependent, e.g., on the temperature and other parameters. The secondary drying time is dictated by the desired residual moisture level in the product and typically takes at least about 5 hours (e.g. 10-15 hours). The pressure may be the same as that employed during the primary drying step. Freeze-drying conditions can be varied depending on the formulation and vial size.

In some instances, it may be desirable to lyophilize the protein formulation in the container in which reconstitution of the protein is to be carried out in order to avoid a transfer step. The container in this instance may, for example, be a 3, 5, 10, 20, 50 or 100 cc vial. As a general proposition, lyophilization will result in a lyophilized formulation in which the moisture content thereof is less than about 5%, and preferably less than about 3%.

At the desired stage, typically when it is time to administer the protein to the patient, the lyophilized formulation may be reconstituted with a diluent such that the protein concentration in the reconstituted formulation is preferably similar to that of the pre-lyophilized formulation.

Reconstitution generally takes place at a temperature of about 25° C. to ensure complete hydration, although other temperatures may be employed as desired. The time required for reconstitution will depend, e.g., on the type of diluent, amount of excipient(s) and protein. Exemplary diluents include sterile water, bacteriostatic water for injection (BWFI), a pH buffered solution (e.g. phosphate-buffered saline), sterile saline solution, Ringer's solution or dextrose solution. The diluent optionally contains a preservative. Exemplary preservatives have been described above, with aromatic alcohols such as benzyl or phenol alcohol being the preferred preservatives. The amount of preservative employed is determined by assessing different preservative concentrations for compatibility with the protein and preservative efficacy testing. For example, if the preservative is an aromatic alcohol (such as benzyl alcohol), it can be present in an amount from about 0.1-2.0% and preferably from about 0.5-1.5%, but most preferably about 1.0-1.2%.

Alternatively, a non-lyophilized formulation may be used, including ADCs and any of the well-known carriers, excipients, buffers, stabilizers, preservatives, adjuvants and other additives described herein and well known in the art.

### Dosages and Administration

The ADC formulations described above can be administered to a subject (e.g., a human) in need of the treatment via a suitable route, such as administration by intravenous, intramuscular, intraperitoneal, intracerebrospinal, subcutaneous, intracutaneous, intraarticular, intrasynovial, intrathecal, intradermal, intratumoral, intranodal, intramedulla, oral, inhalation or topical routes;

or it may be administered orally, by inhalation spray, topically, rectally, nasally, buccally, vaginally or via an implanted reservoir; and in any case, as a bolus or by continuous infusion over a period of time; or via injectable depot routes of administration such as using 1-, 3-, or 6-month depot injectable or biodegradable materials and methods.

Commercially available nebulizers for liquid formulations, including jet nebulizers and ultrasonic nebulizers are useful for administration. Liquid formulations can be directly nebulized and lyophilized powder can be nebulized after reconstitution. Alternatively, ADCs can be aerosolized using a fluorocarbon formulation and a metered dose inhaler, or inhaled as a lyophilized and milled powder. Where CAR is deployed in the invention, compositions of immune effector cells (e.g., T cells, NK cells) may be injected directly into a tumor, lymph node, or site of infection, or elsewhere.

The subject to be treated by the methods described herein can be a mammal, more preferably a human. Mammals include, but are not limited to, farm animals, sport animals, pets, primates, horses, dogs, cats, mice and rats.

An “effective amount” refers to the amount of ADC required to confer therapeutic effect on the subject, either alone or in combination with one or more other active agents. Effective amounts vary, depending on the particular condition being treated, the severity of the condition, the individual patient parameters including age, physical condition, size, gender and weight, the duration of the treatment, the nature of concurrent therapy (if any), the specific route of administration and like factors, all of which are well known to those of ordinary skill in the art and can be addressed with no more than routine experimentation. It is generally preferred that a maximum dose of the individual components or combinations thereof be used, that is, the highest safe dose according to sound medical judgment. A lower dose or tolerable dose for medical reasons, psychological reasons or other reasons, is also appropriate.

Empirical considerations, such as the ADC half-life, generally will contribute to the determination of the dosage. For example, ADCs that are compatible with the human immune system, e.g., those including humanized antibodies or fully human antibodies, may be used to prolong half-life of the antibody and to prevent the ADC being attacked by the host's immune system. Frequency of administration may be determined and adjusted over the course of therapy, and is generally, but not necessarily, based on treatment and/or suppression and/or amelioration and/or delay of cancer. Alternatively, sustained continuous release formulations of antibody may be appropriate. Various formulations and devices for achieving sustained release are known in the art.

In one example, dosages for an ADC as described herein may be determined empirically in individuals who have been given one or more administration(s) of the antibody. Individuals are given incremental dosages of the antibody. To assess efficacy of the ADC, an indicator of the disease (e.g., tumor growth) can be followed according to routine practice.

Generally, for administration of any of the ADCs described herein, an initial candidate dosage can be extrapolated from the experiments described below. For repeated administrations over several days or longer, depending on the condition, the treatment is sustained until a desired suppression of symptoms occurs or until sufficient therapeutic levels are achieved to alleviate the cancer. An exemplary dosing regimen comprises administering an initial higher dose, followed by a lower maintenance dose. However, other dosage regimens may be useful, depending on the pattern of pharmacokinetic decay that the practitioner wishes to achieve. For example, dosing from one-four times a week is contemplated. In some embodiments, dosing frequency is once every week, every 2 weeks, every 3 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

Conventional methods, known to those of ordinary skill in the art of medicine, can be used to administer the pharmaceutical composition to the subject, depending upon the treatment goal and the cancer site.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like).

For intravenous injection, water soluble ADCs can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients.

Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an ADC is administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., WO 00/53211 and U.S. Pat. No. 5,981,568.

In another embodiment of the present disclosure, an article of manufacture is provided which contains any of the pharmaceutical compositions and formulations described herein (e.g., comprising an ADC) and provides instructions for its use and/or reconstitution. The article of manufacture comprises a container. Suitable containers include, for example, bottles, vials (e.g., dual chamber vials), syringes (such as dual chamber syringes) and test tubes. The container may be formed from a variety of materials such as glass or plastic. The container holds the formulation and the label on, or associated with, the container may indicate directions for reconstitution and/or use. For example, the label may indicate that the formulation is reconstituted to particular protein concentrations. The container holding the formulation may be a multi-use vial, which allows for repeat administrations (e.g., from 2-6 administrations) of the reconstituted formulation. The article of manufacture may further comprise a second container comprising a suitable diluent (e.g., BWFI). Upon mixing of the diluent and the lyophilized formulation, the final protein concentration in the reconstituted formulation will generally be at least 50 mg/mL. The article of manufacture may further include other materials desirable from a commercial and user standpoint, including other buffers, diluents, filters, needles, syringes, and package inserts with instructions for use.

### EXAMPLES

The following Examples are not limiting but only exemplary. Unless otherwise specified, all cell lines were purchased from American Type Culture Collection (ATCC, Manassas, VA) and cultured in Dulbecco s Modified Eagle's Medium (DMEM; Thermo Fisher Scientific, Waltham, MA) supplemented with 10% (v/v) Gibco fetal bovine serum (FBS; Thermo Fisher Scientific) at 37° C. in a humidified atmosphere with 5% CO₂. Standard methods were used to manipulate DNA and RNA described in Sambrook et al., Molecular cloning: A laboratory manual; Cold Spring Harbor

Laboratory Press, Cold Spring Harbor, New York, 1989. All commercial reagents and kits were used according to the manufacturer's instructions unless otherwise stated.

### Example 1

### Generation and Selection of MAb52-29.1 mAb and MAb52-25.1 mAb Specifically Bound to Human Cancer Cell Lines

### A. Generation of Hybridoma Clones by Live-Cell Immunization and Live-Cell Screening

To obtain mAbs specifically targeting human malignant tumors, a mixture of three triple-negative breast cancer (TNBC) cell lines, BT-20, BT549, and Hs 578T, was used for live-cell immunization (LCI) and live-cell high-throughput screening (LC-HTS) as described previously (see: U.S. Pat. No. 11,939,391B2, US20210139602A1, WO2014146487A1, WO2017114204). Hybridoma culture supernatants were screened for binding to the surfaces of these three cell lines and counter-screened with human peripheral blood mononuclear cells (PBMCs) from healthy adult donors (Stanford Blood Center, Palo Alto, CA) using a fluorescence-activated cell sorting (FACS) assay. Briefly, cancer cell lines cultured in DMEM/10% (v/v) FBS were detached using 0.2% (w/v) Ethylenediaminetetraacetic acid (EDTA) and mixed with PBS. The cells were collected by centrifugation at 500×g and blocked with ice-cold PBS containing 1.0% (w/v) bovine serum albumin (BSA; Thermo Fisher Scientific). Hybridoma culture supernatants were incubated with the cells at 4° C. for 30 minutes (min). Surface antibody binding was detected using 1:800 diluted Fluorescein AffiniPure™ Goat Anti-Mouse IgG, Fcγ Fragment Specific (Jackson ImmunoResearch Laboratories, West Grove, PA). After three washes with ice-cold PBS/1.0% (w/v) BSA, cells were fixed with 4.0% (w/v) paraformaldehyde. FACS was performed using BD FACSCalibur™ Flow Cytometer equipped with High Throughput Sampler (HTS), and data were analyzed using FlowJo™ 10.8 software (Becton Dickinson, San Jose, CA).

Hybridoma colonies that exhibited high fluorescence signals in binding to TNBC cell lines, but very weak or no binding signal to PBMCs in the FACS assay, were selected for expansion, weaning from conditional medium, and subcloning following standard protocols (Kohler & Milstein, Nature 1975, 256:495-497; Winter & Milstein, Nature 1991, 349:293-299). MAbs secreted by these hybridoma subclones were purified from the culture supernatants by affinity chromatography using MabSelect™ SuRe™ LX Protein A resin (Cytiva, Wilmington, DE) and buffer-exchanged into phosphate-buffered saline (PBS, pH7.2; Cytiva). Antibody concentrations were determined by absorbance at 280 nM (A280) using a Nanodrop™ 2000 Spectrophotometer (Thermo Fisher Scientific). The purity and integrity of the mAbs were assessed by sodium dodecyl-sulfate polyacrylamide gel electrophoresis (SDS-PAGE) on 4-12% SurePAGE™ Bis-Tris gels (GenScript, Piscataway, NJ) stained with SimplyBlue™ SafeStain (Thermo Fisher Scientific). The isotype of each mAb was determined using IsoStrip™ Mouse Monoclonal Antibody Isotyping Kit (Roche Molecular Systems, Pleasanton, CA).

B. FACS Analysis of mAbs in Binding to Cancer Cell Surfaces

The reactivity of purified mAbs was confirmed by assessing their binding to representative cancer cell lines from different cancer types with FACS assays. Purified mAbs with serial dilutions were incubated with individual cell lines at 4° C. for 30 min, followed by detection with Fluorescein AffiniPure™ Goat Anti-Mouse IgG, Fcγ Fragment Specific. To evaluate specificity and potential off-target effects, multiple batches of fresh PBMCs from distinct healthy donors were stained in parallel as negative controls. Additionally, frozen human bone marrow mononuclear cells (BMMCs) from healthy donors and human cord blood mononuclear cells (CB-MNCs) purchased from HumanCells Bioscience (Milpitas, CA) were carefully thawed and stained to assess the safety profile of the mAbs as therapeutic candidates.

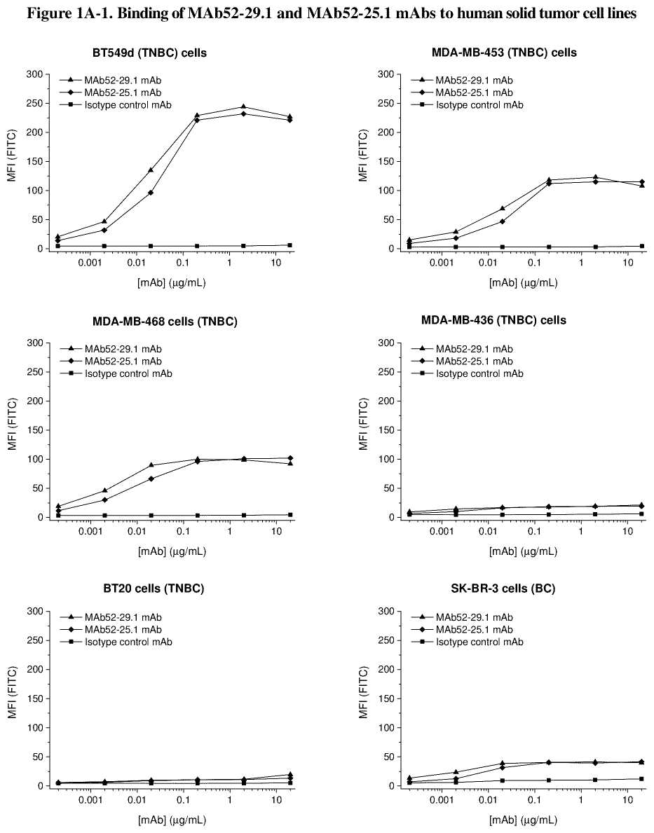

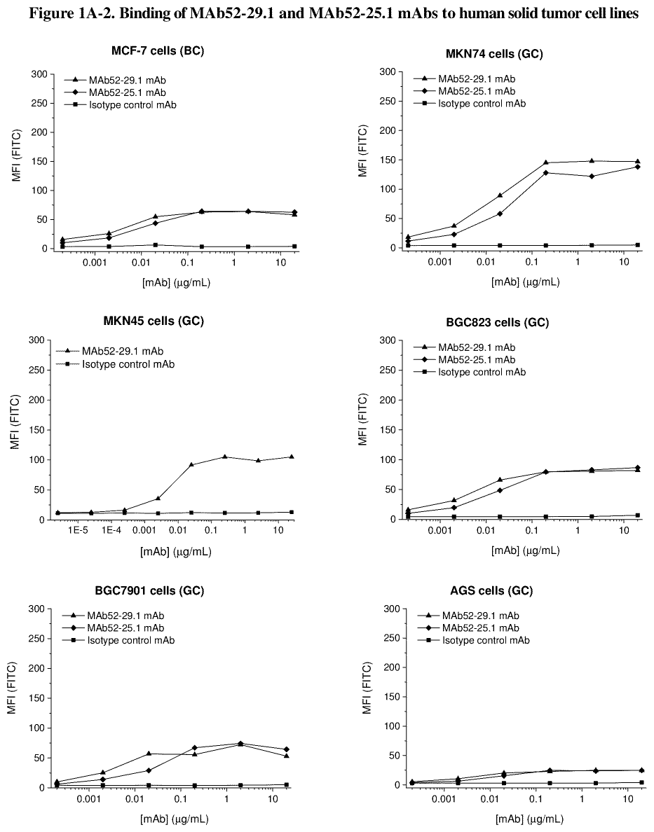

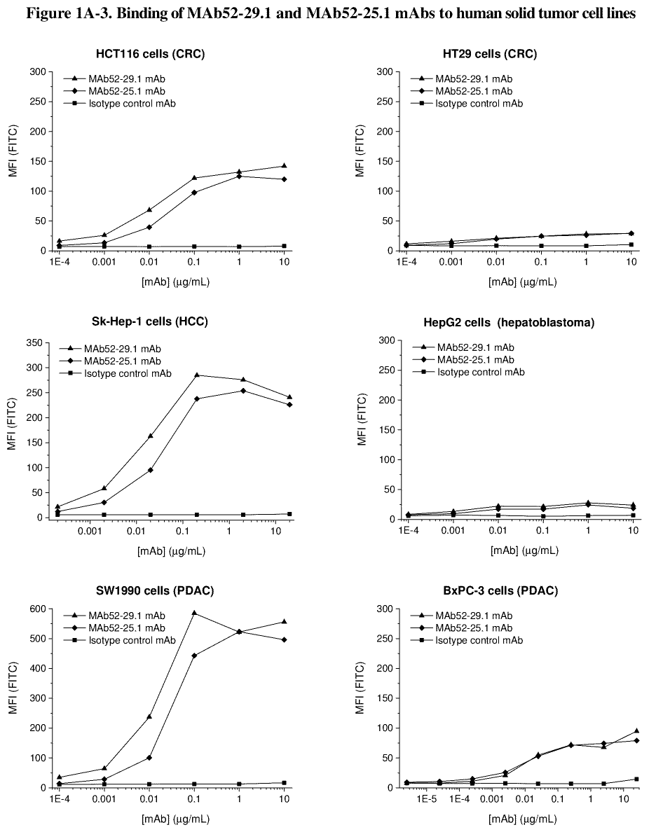

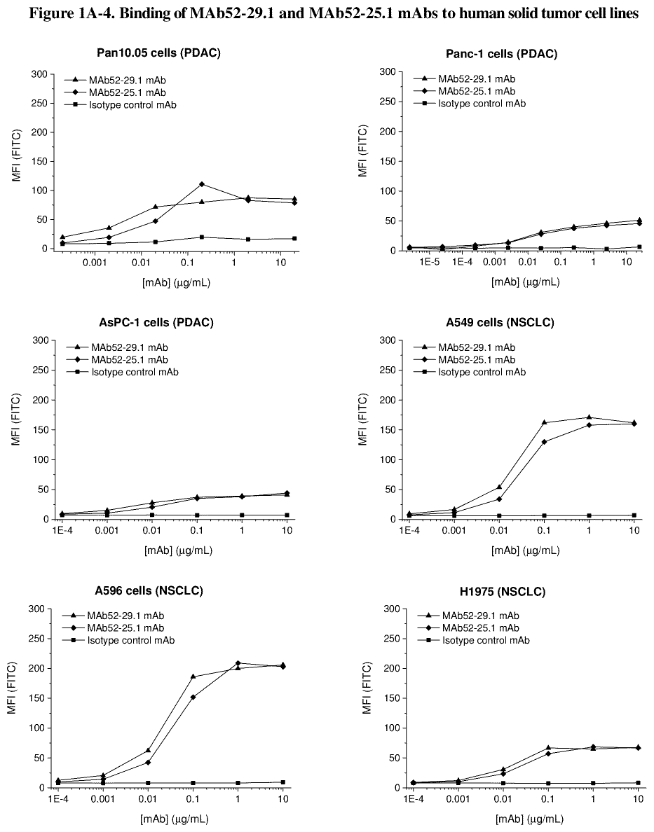

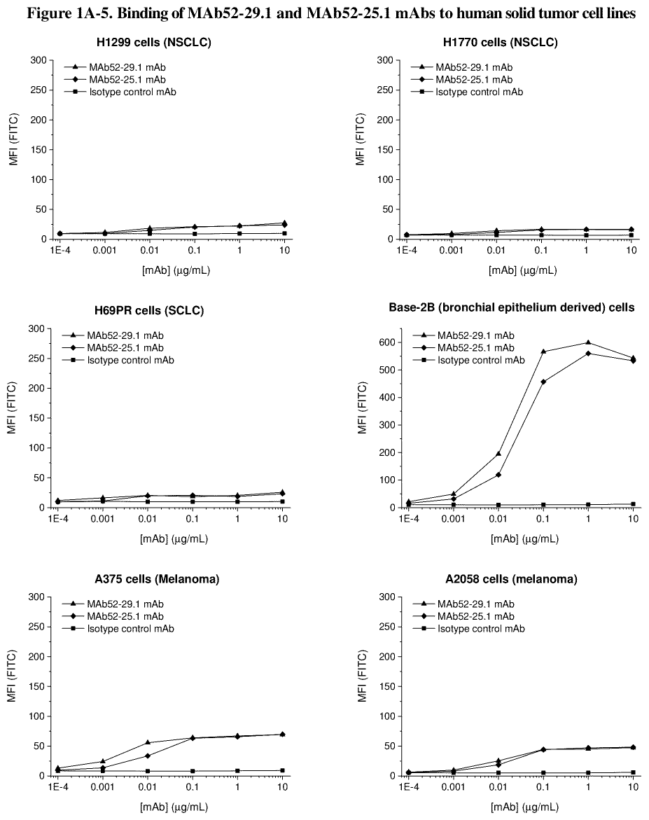

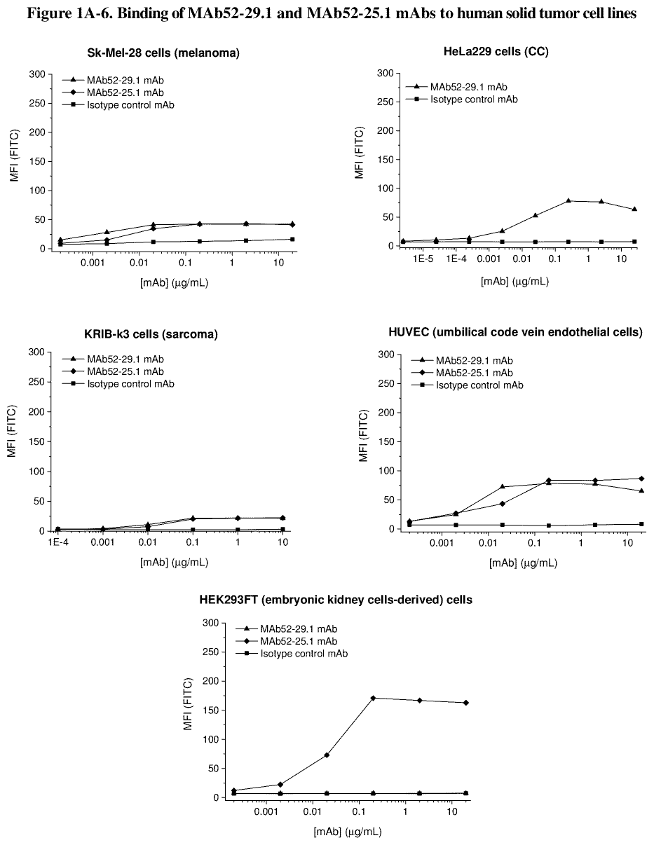

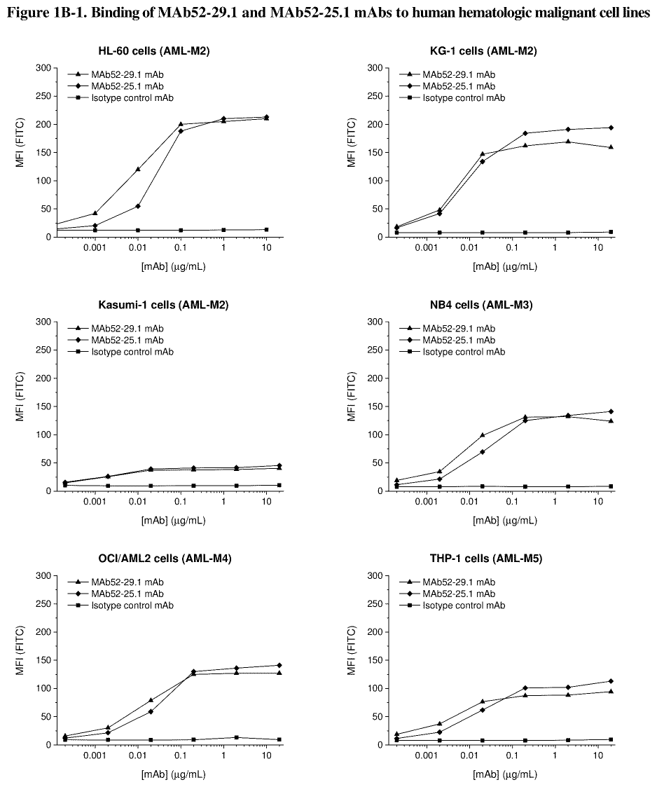

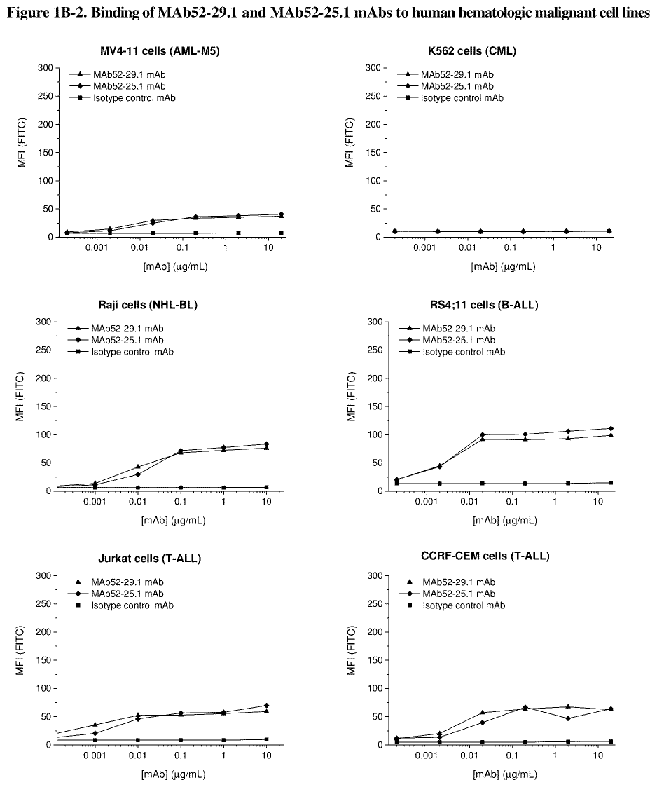

Among all purified mAbs, MAb52-29.1 mAb and MAb52-25.1 mAb exhibited high binding capacities to a majority of the cancer cell lines tested (FIG. 1A and FIG. 1B), suggesting that their targets are widely expressed across both solid tumors and hematologic malignancies, and may play a role in cancer cell proliferation and metastasis. MAb52-29.1 mAb showed a slower off-rate than MAb52-25.1 mAb in binding to many cancer cell lines, implicating a slightly higher binding affinity. MAb52-25.1 mAb reacted with HEK293FT cells, which did not express the target epitope of MAb52-29.1 mAb.

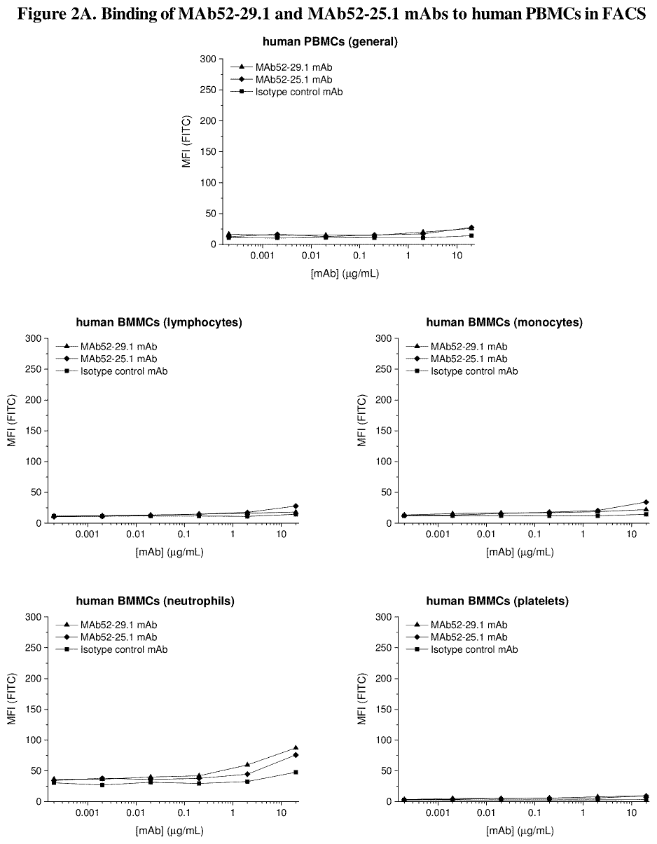

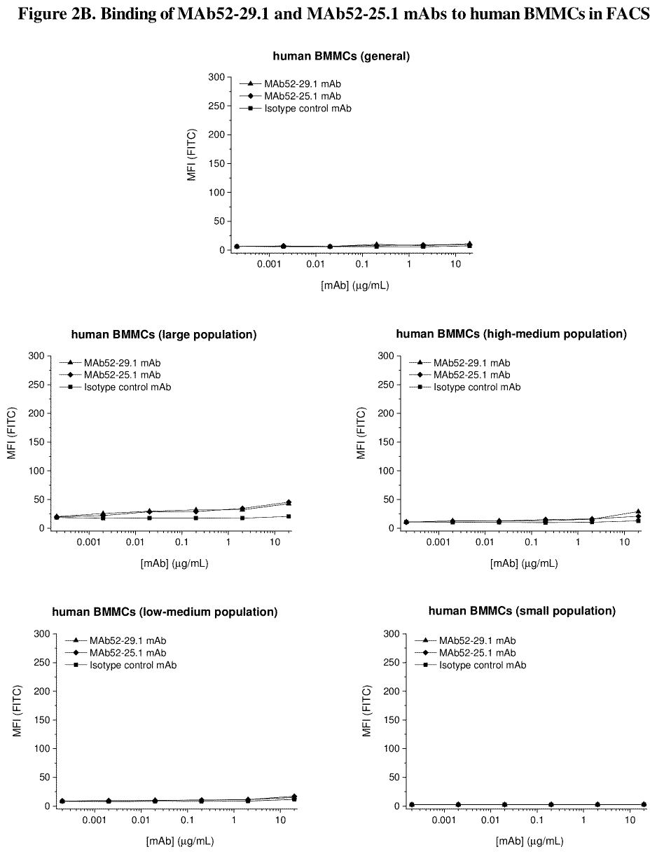

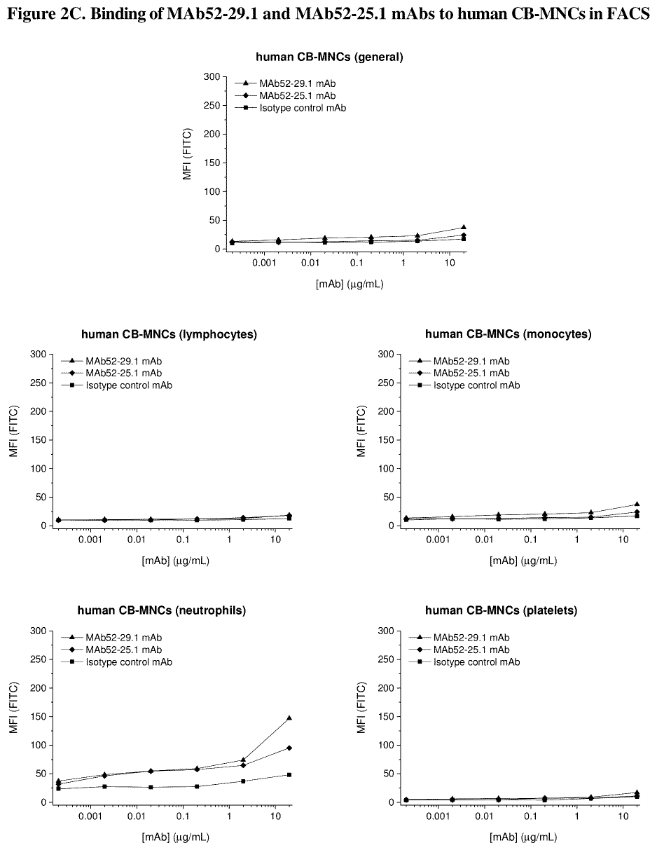

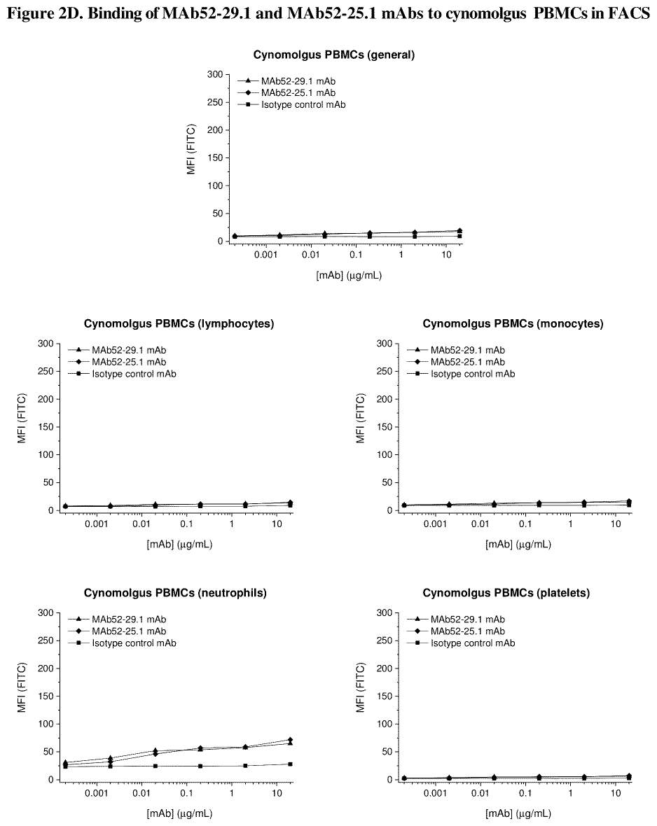

In FACS analyses with normal human cells, both mAbs exhibited very weak binding, primarily to what appeared to be neutrophils in human PBMCs (FIG. 2A) and CB-MNCs (FIG. 2C), and the large-cell subpopulation of BMMCs (FIG. 2B). Their interactions with CB-MNCs were slightly stronger, suggesting that their targets may be expressed in early-stage cells (FIG. 2C). Additionally, both mAbs showed slight cross-reactivity with what were likely neutrophils isolated from cynomolgus monkeys, displaying a slightly higher affinity than their binding to human neutrophils (FIG. 2D). This finding suggests that the targets may be conserved between humans and monkeys. Alternatively, the observed binding to neutrophils could be non-specific, given the complex matrix structures present on the surface of these cells.

Isotype typing revealed that both MAb52-29.1 and MAb52-25.1 mAbs consist of murine kappa light chains and IgG1 heavy chains, and therefore, a murine IgG1-Kappa isotype control mAb was included in all binding assays for these mAbs thereafter.

### C. IHC Assay of MAb52-29.1 mAb in Binding to Normal Human Tissues

An immunohistochemistry (IHC) assay was performed using FDA standard formalin-fixed paraffin-embedded (FFPE) tissue microarray (TMA) slides (Biochain, Newark, CA) to evaluate whether MAb52-29.1 mAb cross-reacts with normal human tissues. Each TMA slide contains 30 different human tissue samples, each donated from three distinct donors. Among them, 29 are normal tissues, and the placenta sections serve as a positive control. Lung cancer (NSCLC) FFPE sections were used to validate the IHC protocol and the optimal concentration of MAb52-29.1 mAb for the assay. Tissue sections were deparaffinized, rehydrated through graded alcohols, and subjected to heat-induced antigen retrieval (HIER), followed by blocking with 10% (v/v) horse serum (Vector Laboratories, Newark, CA). Sections were incubated overnight at 4° C. with 100 μg/mL MAb52-29.1 mAb. Antibody binding was detected using VECTASTAIN® Elite® ABC-HRP Kit, Peroxidase (Mouse IgG) from Vector Laboratories. Samples were counterstained with Mayer's hematoxylin solution (MilliporeSigma, St. Louis, MO), mounted in VectaMount® AQ Mounting Medium (Vector Laboratories), and coverslipped. Tissue sections were imaged and scanned using the BZ-X800 All-in-One Fluorescence Microscope (Keyence, Itasca, IL).

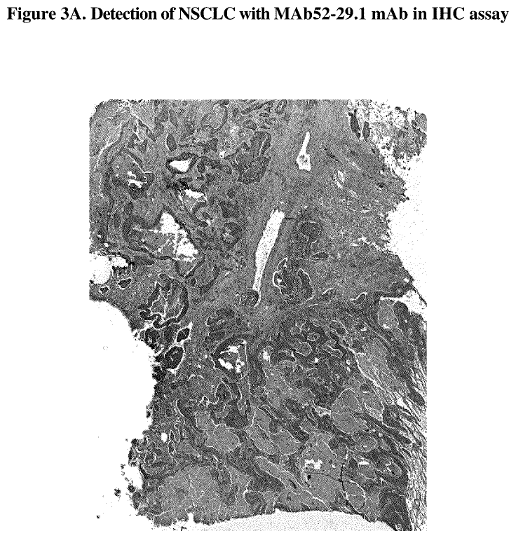

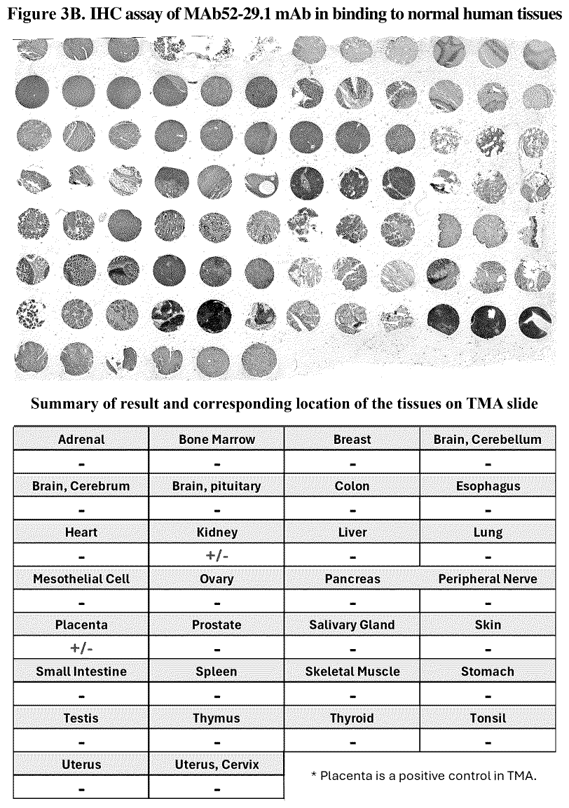

IHC analysis revealed that lung cancer tissue sections stained with 100 μg/mL (or 0.67 μM) of MAb52-29.1 mAb exhibited a strong brown precipitate, indicating high expression levels of the target antigen (FIG. 3A). In contrast, MAb52-29.1 mAb showed no staining or only weak background signals in 28 of 29 normal tissue types examined on the normal TMA slide (FIG. 3B). The only exception was the kidney, where weak signals were observed. However, this was likely due to the reaction between endogenous biotin in the kidney and the horse radish peroxidase (HRP) conjugated to the secondary antibody, a common artifact observed in IHC with therapeutic antibodies.

These findings confirm a clear distinction in target protein expression between cancerous and normal tissues, highlighting the potential of MAb52-29.1 mAb, and presumably MAb52-25.1 mAb, as promising therapeutic candidates for cancer diagnosis and treatment.

### Example 2

### Identification and Verification of Human CD166 as the Target of MAb52-25.1 mAb and MAb52-29.1 mAb

### A. Target Identification by Immunoprecipitation (IP) and Mass Spectrometry (MS)

To identify the target of MAb52-25.1 mAb, the mAb was conjugated to Dynabeads™ Protein-A (Thermo Fisher Scientific) for immunoprecipitation (IP), or to HRP with Lightning-Link® HRP Conjugation Kit (Abcam, Waltham, MA), according to the manufacturers' protocols. Cancer cell lines were lysed with radioimmunoprecipitation assay (RIPA) buffer (Thermo Fisher Scientific), and the lysates of mixed cancer cell lines were incubated with the Dynabeads™-mAb complex at room temperature (RT) for 30 min. The complex was washed sequentially with 0.5% (v/v) TritonX-100/PBS and PBS. The immunoprecipitated (IP) protein samples were separated by SDS-PAGE under a non-reducing condition and transferred to a nitrocellulose membrane (MilliporeSigma). The membrane was blocked with PBS containing 0.05% (v/v) Tween®-20 (PBST) and 1% (w/v) BSA and probed with MAb52-25.1 mAb-HRP, followed by detection using SuperSignal™ West Pico PLUS Chemiluminescent Substrate (Thermo Fisher Scientific).

Nevertheless, MAb52-25.1 mAb-HRP failed to detect any specific bands in the Western blot analysis, suggesting that its target is likely a conformational epitope that was denatured during the Western blot process. This indicates that this mAb most likely recognizes a three-dimensional structure rather than linear peptide sequences. To further investigate, the IP samples were digested directly with trypsin and Lys-C protease and analyzed by liquid chromatography with tandem mass spectrometry (LC-MS/MS, Innomics, San Jose, CA). The resulting data were searched against the latest reviewed human database from Uniprot (https://www.uniprot.org/) using Mascot. Recombinant proteins were purchased to assess their interaction with the mAbs using ELISA assays.

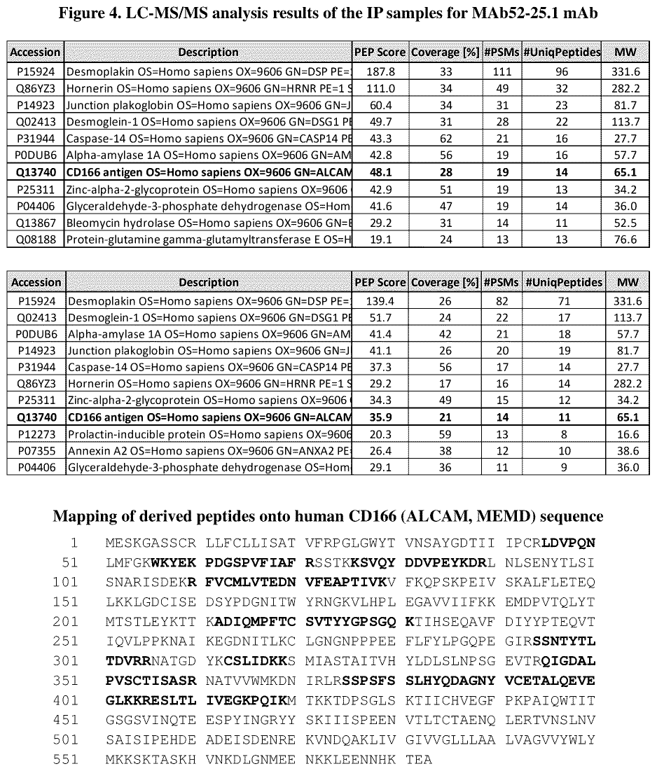

FIG. 4 lists the most confidently identified proteins detected from the MAb52-25.1 mAb-precipitated samples by LC-MS/MS. CD166, also known as ALCAM (UniProtKB/Swiss-Prot: Q13740), had 28% sequence coverages was purchased for ELISA assays. Other proteins identified were excluded based on their properties and subcellular locations.

### B. Binding of Both MAb52-25.1 mAb and MAb52-29.1 mAb to Human CD166 in ELISA Assays

An ELISA assay using a recombinant human CD166 extracellular domain (ECD, Trp28-Ala526) with a C-terminal His tag, namely rhCD166-ECD-His, expressed in HEK293 cells (Sino Biological US, Wayne, PA) was performed to assess its reactivity with MAb52-25.1 mAb as well as MAb52-29.1 mAb, due to their similar binding pattern to cell lines. The antigen was immobilized on Immulon® microtiter plates (Thermo Fisher Scientific) at 15 ng/well, washed with PBST and blocked with PBST/1% (w/v) BSA at RT for one hour. MAb52-29.1 mAb, MAb52-29.1 mAb, anti-6×His-tag mAb (Proteintech, Rosemont, IL), or an isotype control mAb was added in serial dilutions and incubated for one hour. After washing, the plates were probed with 1:4,000 diluted Peroxidase AffiniPure™ Goat Anti-Mouse IgG, Fcγ fragment specific (Jackson ImmunoResearch Laboratories) for 30-60 min. Following thorough wash, 3,3′,5,5′-Tetramethylbenzidine (TMB) was added to allow color development for 15 min and then stopped with 0.05 M sulfuric acid. Absorbance at 450 nm (A450) was measured using the SpectraMax Microplate Reader (Molecular Device, San Jose, CA).

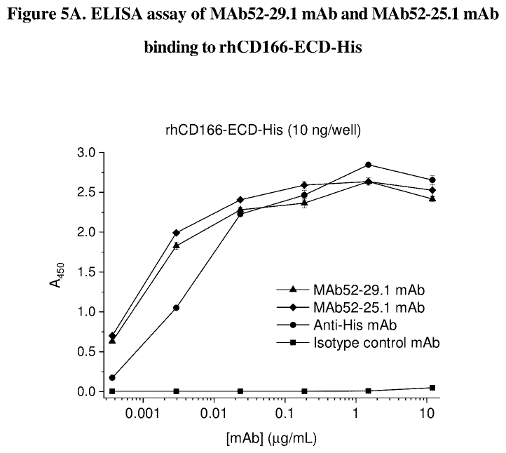

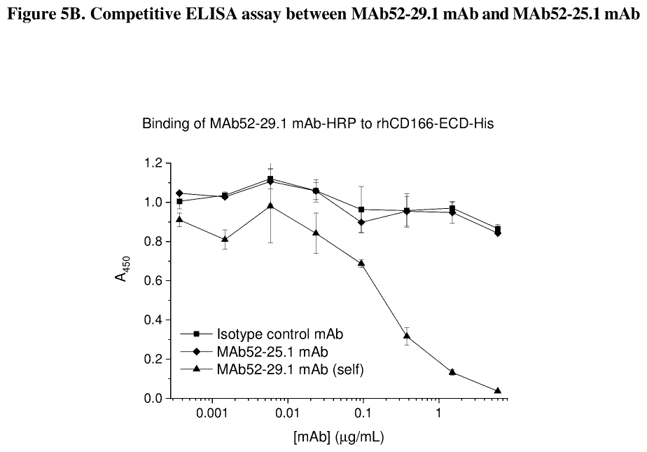

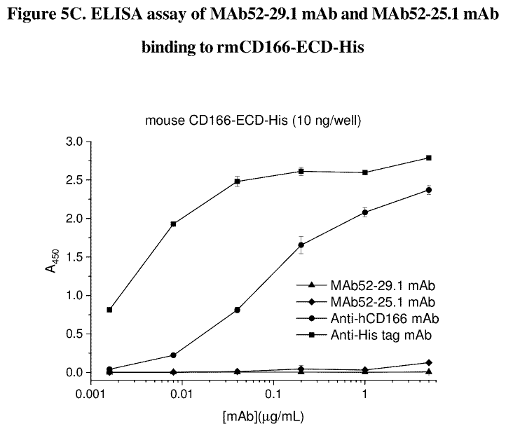

Both MAb52-25.1 mAb and MAb52-29.1 mAb showed strong dose-dependent binding curves to rhCD166-ECD-His with similar affinities, with EC₅₀=0.01 μg/mL or 0.067 nM (FIG. 5A). This ELISA result confirmed the LC-MS/MS result of CD166 as the target of MAb52-25.1 mAb, and also indicated that MAb52-29.1 mAb recognized the same target as MAb52-25.1 mAb.

The slight difference in binding affinities to cancer cell lines and striking difference in binding to HEK293FT cells by these two mAbs implicated that they recognize different epitopes on CD166. To confirm this hypothesis, a competitive ELISA assay was performed using MAb52-25.1 mAb, MAb52-29.1 mAb, or the isotype control cAbm2 in 3-fold serial dilution to mix with 1:4,000 diluted MAb52-29.1mAb-HRP, and incubated with immobilized rhCD166 (ALCAM) for one hour. After thorough washing, TMB substrate was added to detect binding signals between MAb52-29.1mAb-HRP and the antigen.

As shown in FIG. 5B, the isotype control mAb, which targets an irrelevant antigen, served as the baseline for MAb52-29.1mAb-HRP and rhCD166-ECD-His binding. MAb52-29.1 mAb, as a positive control of self-competition exhibited a dose-dependent reduction in binding signals. MAb52-25.1 mAb showed a nearly overlapping curve as the isotype control mAb, confirming that MAb52-25.1 mAb and MAb52-29.1 mAb bind to non-overlapping epitopes of CD166.

The human CD166 protein shares 99% and 93% sequence identities with cynomolgus monkey and mouse CD166, respectively. Both MAb52-25.1 and MAb52-29.1 mAbs interacted weakly with the subpopulation appeared to be neutrophils in monkey peripheral blood (FIG. 2D), suggesting their targeting epitopes also present in monkeys.

To explore whether these epitopes also exist in mouse CD166, an ELISA assay was performed using immobilized recombinant mouse CD166-ECD (rmCD166-ECD-His), which was similarly expressed and purified as rhCD166-ECD-His (Sino Biological US). However, neither MAb52-25.1 mAb nor MAb52-29.1 mAb showed cross-species interaction with rmCD166-ECD-His (FIG. 5C), indicating that their targeting epitopes on human CD166 do not exist in mouse CD166. On contrary, the commercial anti-human CD166 mAb (Clone No. 3A6, Proteintech) bound to an epitope of CD166 shared between human and mouse.

### C. Western Blot Confirming a Conformational Epitope Bound by MAb52-29.1 mAb

For further verify the target, 1.0 μg rhCD166-ECD-His was electrophoresed along with various cancer cell lysates containing approximately 2.0 μg total proteins on a 4-12% SurePAGE™

Bis-Tris gels under non-reducing conditions. After gel-transferring to polyvinylidene fluoride (PVDF) membrane (MilliporeSigma), the proteins were reacted with MAb52-29.1 mAb for one hour, washed, then probed with Peroxidase AffiniPure™ Goat Anti-Mouse IgG (Jackson ImmnoReaserch Laboratories) and HRP-conjugated anti-β-actin mAb (Proteintech), followed by reacting with SuperSignal™ West Pico PLUS Chemiluminescent Substrate (Thermo Fisher Scientific).

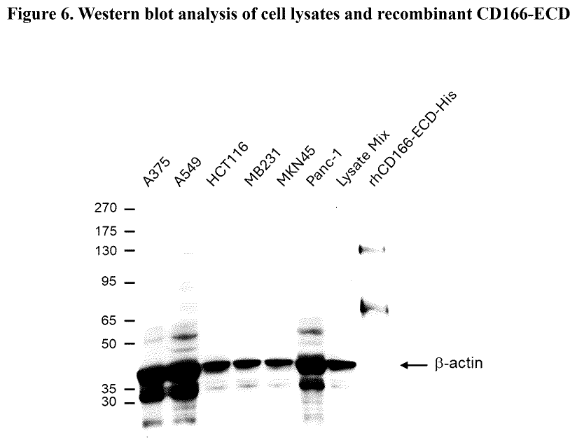

As expected, MAb52-29.1 mAb failed to detect any specific band in cancer cell lysates, likely due to denaturation of the conformational epitope during electrophoresis (FIG. 6). Two bands at approximately 80 kDa and 130 kDa were detected in the lane of rhCD166-ECD-His, which has a calculated molecular mass of 57.4 kDa but is expected as an 78-kDa band in SDS-PAGE under reducing condition. Hence, the 80-kDa and 130-kDa bands on the non-reducing gel corresponded well to a glycosylated monomeric and a non-glycosylated dimeric rhCD166-ECD-His, respectively, confirming the direct interaction between MAb52-29.1 mAb and human CD166. Weak detection of rhCD166-ECD-His despite a 1.0 μg loading further supported that MAb52-29.1 mAb binds a conformation-dependent epitope on CD166, which was disrupted by SDS-PAGE sample preparation.

D. Binding Kinetics and Affinity Between MAb52-29.1 mAb and rhCD166-ECD-his Determined by Octet

The binding kinetics and affinity of MAb52-29.1 mAb to rhCD166-ECD-His was determined using Bio-Layer Interferometry (BLI) label-free technology with an Octet® QKe System (Molecular Devices, San Jose, CA). Anti-Mouse IgG Fc Capture (AMC) Biosensors (Sartorius, Fremont, CA) were immobilized with 100 nM MAb52-29.1 mAb for 10 min, followed by a 5-min wash with 1x Kinetic Buffer (Sartorius). After the baseline stabilized, sensors loaded with MAb52-29.1 mAb were exposed to rhCD166-ECD-His for 15 min to obtain the association curve, followed by 30 min of dissociation in 1×Kinetic Buffer. The assay was repeated multiple times using 2-fold serial dilutions of rhCD166-ECD-His, ranging from 100 nM to 6.25 nM, in 1x Kinetic Buffer. Kinetic data were processed with average referencing and fit to a 1:1 interaction model using Octet® Data Analysis software (version 10) to determine the association rate constant (kₒₙ) and dissociation rate constant (k_{off} or k_{dis}). The equilibrium dissociation constant (K_{D}) in molar units (M) was calculated using the formula K_{D}=k_{off}/kₒₙ. The biosensors were regenerated with 10 mM glycine, pH 1.70.

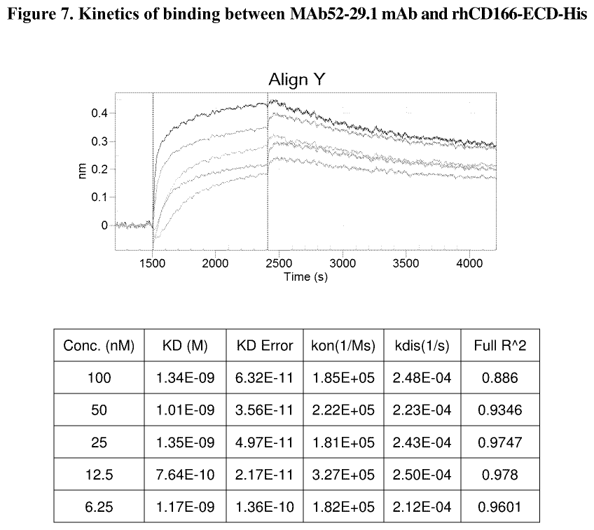

The aligned sensorgram illustrating the association and dissociation between MAb52-29.1 mAb and rhCD166-ECD-His is shown in FIG. 7. A fast association rate and a slow dissociation rate suggest a high binding affinity. The affinity (K_{D}) calculated was approximately 1.12×10⁻⁹ M or 1.12 nM.

E. Detection of ALCAM Gene Expression in Cancer Cell Lines by qRT-PCR

CD166 is known to widely expressed in epithelial cells, neuronal cells, fibroblasts, endothelial cells, keratinocytes, immune cells, and stem cells. However, MAb52-29.1 mAb and MAb52-25.1 mAb exhibited no binding to normal PBMCs, BMMCs, and CB-MNCs, except the cells that were likely neutrophils. Additionally, MAb52-29.1 mAb only cross-reacted with kidney, but not with other normal tissues. Therefore, their targeting epitopes on CD166 are highly related to cancer. To assess whether the different expression levels between normal and cancer cells were regulated at the gene or protein levels, the relative expression levels of the ALCAM gene, which encodes CD166, were measured in various cancer cell lines using quantitative reverse transcription-polymerase chain reaction (qRT-PCR).

Six cell lines representing different types of cancer were used for qRT-PCR analysis. These include A375 (melanoma), A549 (NSCLC), BT549d (TNBC), HCT-116 (CRC), MKN45 (GC; AffiGen, Baileys Harbor, WI) cells, and Panc-1 (PDAC) cells, which showed much higher surface binding signals than neutrophils in reaction with MAb52-29.1 mAb or MAb52-25.1 mAb (FIG. 1A and FIG. 2). These cell lines were cultured in DMEM supplemented with 10% (v/v) FBS until reaching exponential phase, detached, and washed with PBS. Total RNA was isolated from the cells using Quick-RNA™ Miniprep Kit (Zymo Research, Irvine, CA), and residual genomic DNA (gDNA) was removed by DNase I digestion (Thermo Fisher Scientific) according to the manufacturer's protocol. First-strand cDNA was synthesized at 25° C. for 10 min, 37° C. for 120 min, and 85° C. for 5 min using the High Capacity cDNA Reverse Transcription Kits (Thermo Fisher Scientific) in a 20-μL reaction containing 1.0 μg of total RNA, 2.0 μL of 10×RT buffer, 4 mM dNTP mix, 2.0 μL of RT Random Primers, 1.0 μL of MultiScribe™ Reverse Transcriptase, and 1.0 μL of RNase Inhibitor. Real-time PCR was performed on the ABI7800 Real Time PCR System (Thermo Fisher Scientific) using Luna Universal qPCR Master Mix (New England Biolabs, Ipswich, MA) in a 20-μL reaction with 0.2 μL of first-strand cDNA as the template. The cycling conditions were as follows: 95° C. for 60 sec, followed by 40 cycles of 95° C. for 15 sec and 60° C. for 30 sec. Relative mRNA expression levels were calculated using the comparative cycle threshold (2^{-ΔΔCT}) method (Livak and Schmittgen, Methods 2001). Ribosomal protein lateral stalk subunit P0 (RPLP0) served as the housekeeping control. The primer sequences for qPCR were:

ALCAM Forward:
(SEQ ID NO: 29)
5′-TCCAGAACACGATGAGGCAGAC-3′
ALCAM Reverse:
(SEQ ID NO: 30)
5′-GTAGACGACACCAGCAACAAGG-3′
RPLP0 Forward:
(SEQ ID NO: 31)
5′-GGCGACCTGGAAGTCCAACT-3′
RPLP0 Reverse:
(SEQ ID NO: 32)
5′-CCATCAGCACCACAGCCTTC-3′

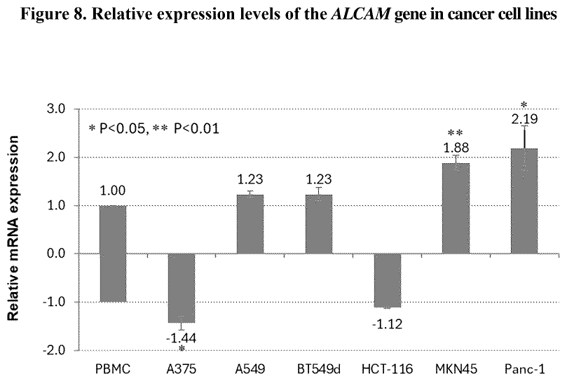

Compared with normal human PBMCs, the ALCAM gene was significantly upregulated in Panc-1 (P<0.01) and MKN45 (P<0.05) cell lines, whereas downregulated in A375 cells (P<0.05) (FIG. 8). No significant difference was observed with A549, BT549d or HCT-116 cells. Thus, the abundance of the targeting epitopes for both MAb52-25.1 mAb and MAb52-29.1 mAb does not correlate with the ALCAM gene expression levels.

### Example 3

### In Vitro Effects of MAb52-29.1 mAb and MAb52-25.1 mAb on Cancer Cell Lines

### A. Cell Invasion Assay of MAb52-29.1 mAb

MAb52-29.1 mAb or MAb52-25.1 mAb did not show significant effect on cancer cell proliferation (data not shown). However, a cell invasion assay using the QCM ECMatrix Cell Invasion Assay Kit (MilliporeSigma) suggested that MAb52-29.1 mAb interferes with cancer cell invasion. This was done with A375, A549, BT549d, HCT-116, MKN45, and Panc-1 cell lines, which were seeded at a density of 3×10⁵ cells/well in serum-free DMEM supplemented with 1.0% (w/v) BSA into the 24-well plate provided by the kit. The cells were treated with 150 μg/mL of MAb52-29.1 mAb or an isotype control mAb and incubated overnight at 37° C. with 5% CO₂ and humidity. Invasive cells that migrated through the ECM layer were stained according to the manufacturer's instructions and quantified by measuring absorbance at 560 nm (A560) using a microplate reader. Each sample was measured in triplicates.

For each cell line, the difference in number of cells that invaded through the ECM matrix between the MAb52-29.1 mAb-treated group (test) and isotype control mAb-treated group (control) was calculated the following formula:

$$Percent change in invasive cells ( \% ) = [ ( {A}_{560} test - {A}_{560} control ) / {A}_{560} control ] \times 100.$$

Isotype control samples were used as the baseline, represented as 0±standard deviation (SD) of triplicated samples. A positive result indicates an increase in cell invasion, whereas a negative result signifies a percentage of reduction in cell invasion.

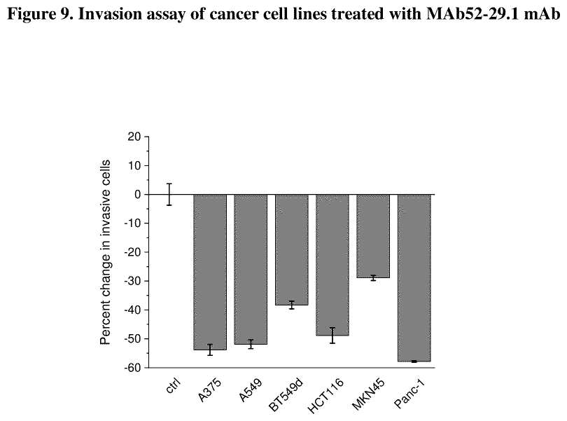

After 24 hours of co-incubation with MAb52-29.1 mAb, all six cell lines exhibited a significant reduction, ranging from 30% to 60%, in the number of cells that migrated across the ECMatrix™ membrane (FIG. 9). Although MAb52-29.1 mAb did not demonstrate cytotoxic effect on cancer cells, it may partially inhibit cancer metastasis.

### B. Internalization Assay of MAb52-29.1 mAb and MAb52-25.1 mAb by Cancer Cell Lines

To assess whether binding of MAb52-29.1 mAb and MAb52-25.1 mAb to the conformational epitopes of CD166 could trigger the internalization of CD166. MAb52-29.1 mAb, MAb52-25.1 mAb, and an isotype control mAb were conjugated with fluorescein (FITC) using Abcam's FITC Conjugation Kit (Abcam). This was done by incubating the mAbs with Modifier Reagent and FITC Mix at RT for 3 hours, followed by quenching of any unbound FITC. Cell lines were cultured in DMEM medium supplemented with 10% (v/v) FBS and plated at optimal densities ranging from 1 to 5×10⁴ cells per well in four Falcon flat-bottomed 96-well tissue culture microplates (Corning, Corning, NY). The cells were incubated overnight at 37° C. with 5% CO₂ and humidity. The cells were pre-cooled to 4° C. and then co-incubated with 0.1 μg/mL FITC-labeled mAb at 4° C. to allow surface binding. One hour later, the cells were rinsed twice with ice-cold PBS to remove the unbound FITC-labeled mAb. One plate was subsequently stained for 15 min at 4° C. with 4′,6-Diamidino-2-Phenylindole, dihydrochloride (DAPI; Biotium, Fremont, CA), rinsed with ice-cold PBS, and then photographed using the BZ-X800 All-in-one Fluorescence Microscope (Keyence, Itasca, IL). Meanwhile, warm culture medium was added to the remaining three plates and incubated at 37° C. for 15 min, 30 min, 2 hours, respectively, to promote internalization of the mAb-antigen complexes. These plates were also stained with DAPI for 15 min at 37° C. prior to PBS wash and imaging.

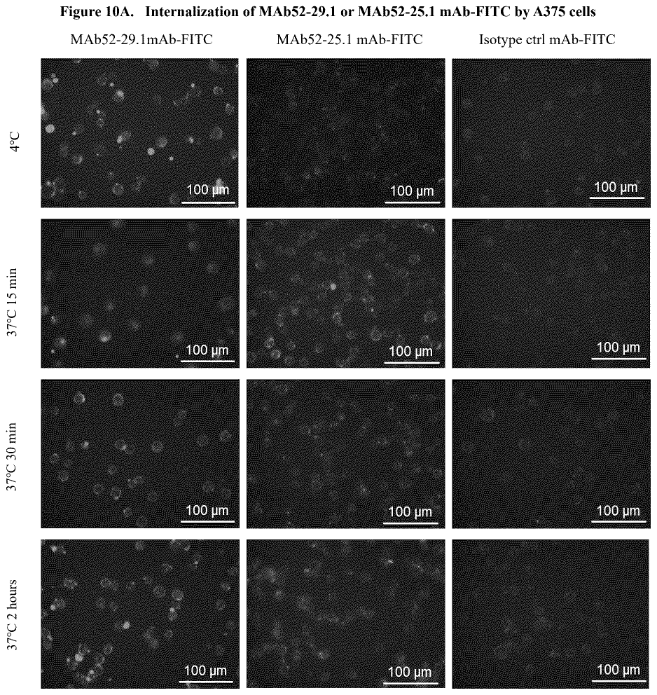

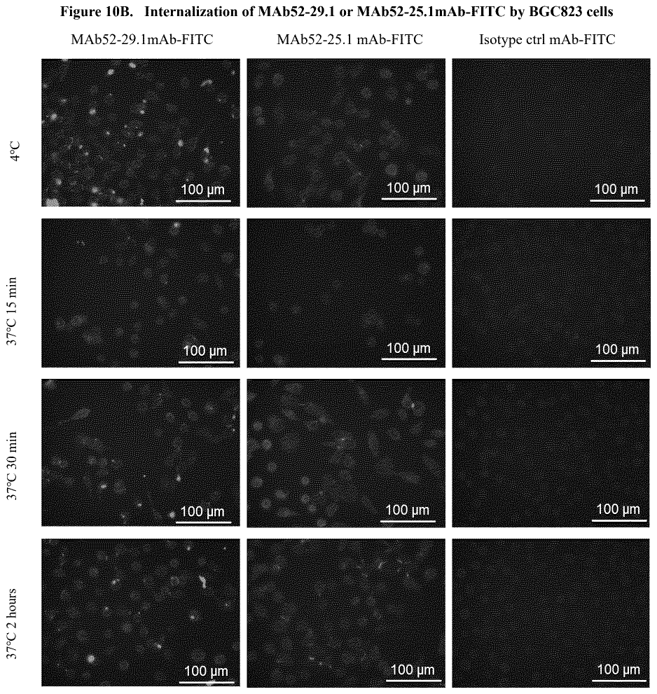

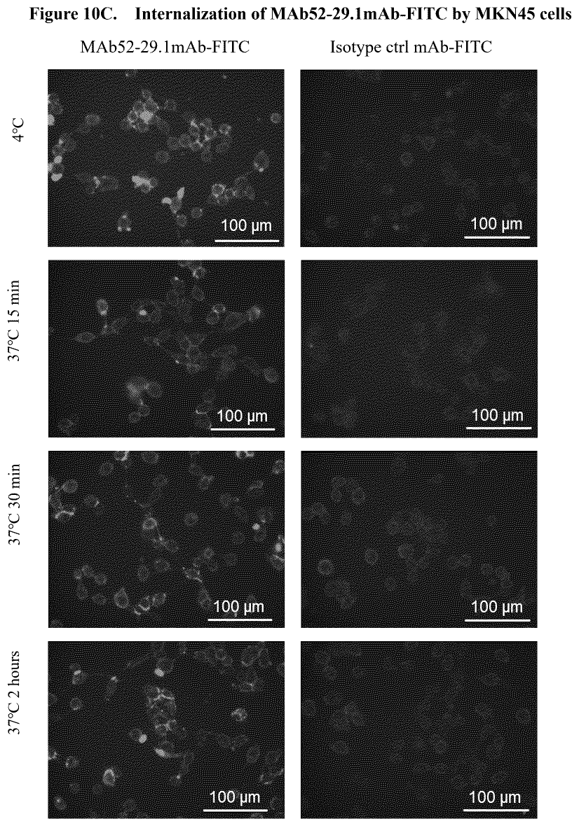

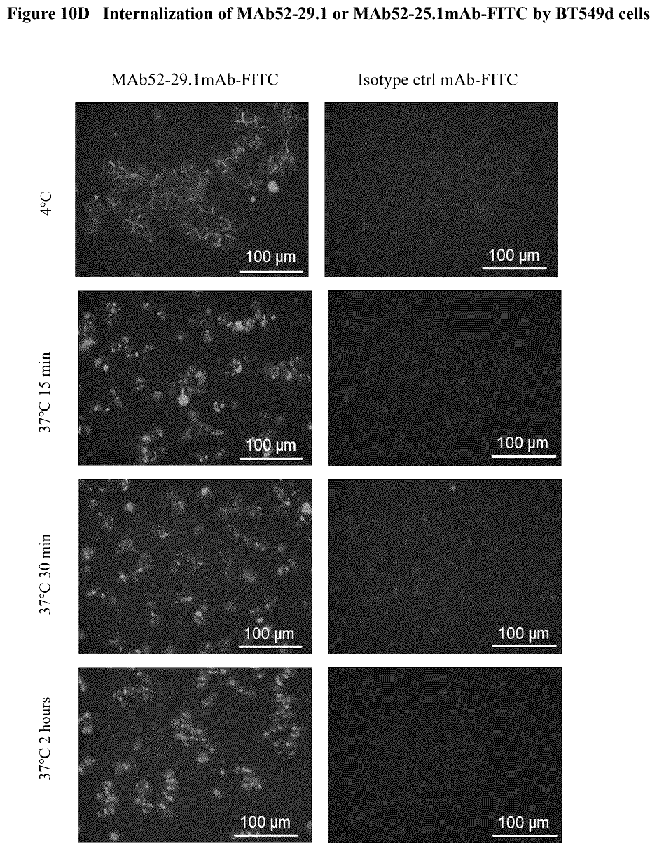

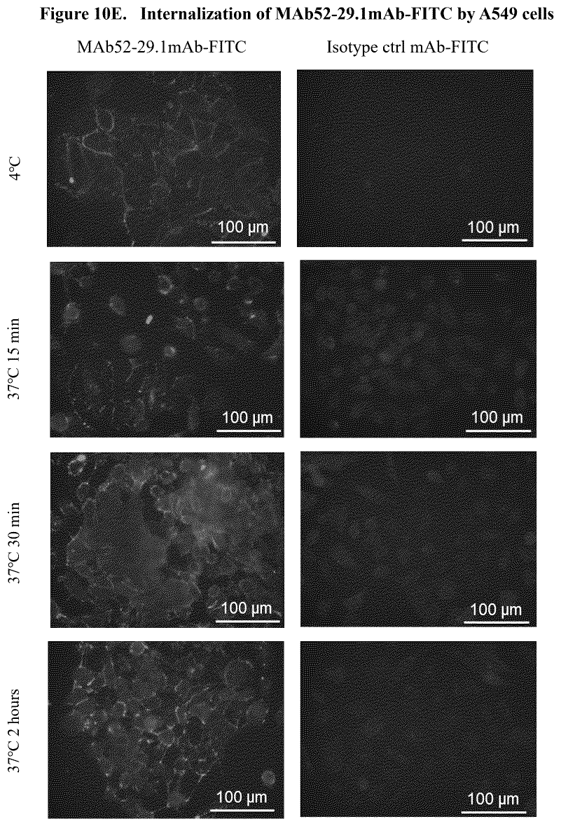

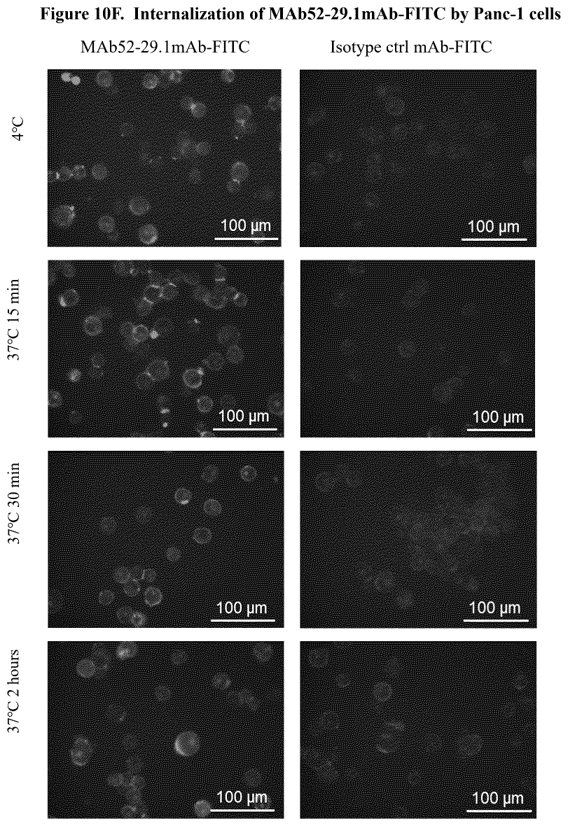

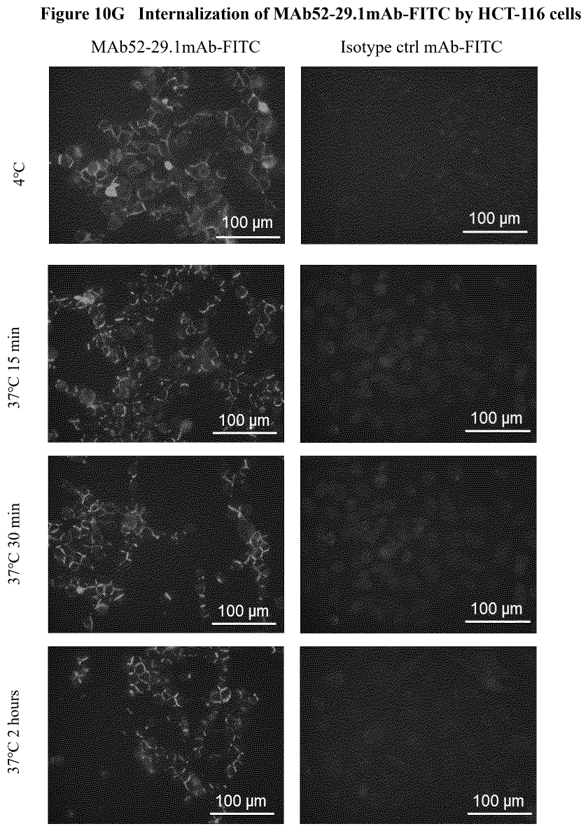

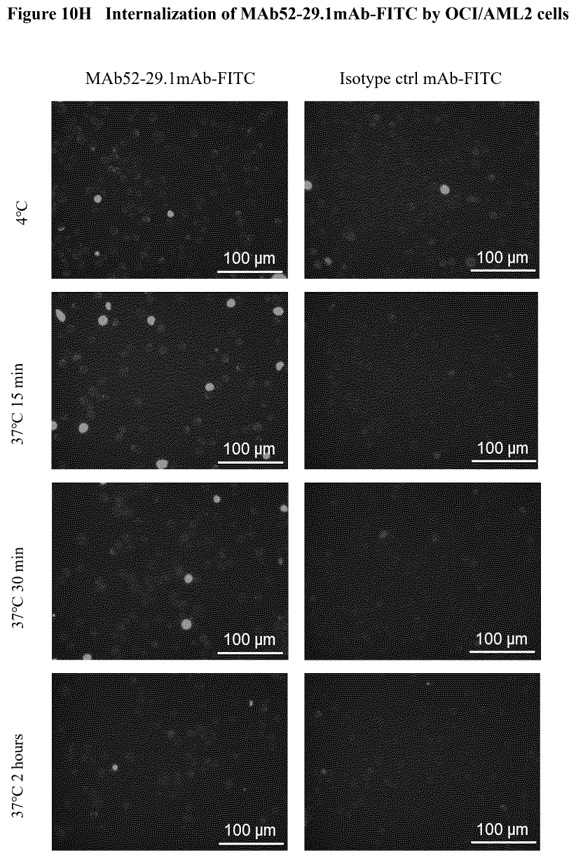

The isotype control mAb-FITC did not bind to any of these cell lines in FACS analysis, but low fluorescence background was observed in A375, MKN45, and Panc-1 cells that were subtracted for evaluating surface binding and internalized (FIGS. 10A to 10H, right panels).

MAb52-29.1mAb-FITC exhibited green fluorescence outlining the cell membrane after one hour of incubation at 4° C., confirming its binding to the target antigen on the cell surface (FIGS. 10A to 10H, left panels). The intensity of surface fluorescence correlated well with the cell staining results shown in FIG. 1A, with BGC823 (GC; AffiGen, WI; FIG. 10B) and OCI/AML2 (FIG. 10H) cells displaying weaker fluorescence than the other cell lines. A375 (FIG. 10A) and BT549d (FIG. 10D) cells showed the highest internalization rate, followed by BGC823 (FIG. 10B), MKN45 (FIG. 10C), and Panc-1 (FIG. 10F) cells, symbolized by the progressive accumulation of intracellular fluorescence as incubation continued at 37° C. from 15 min to 2 hours. A549 (FIG. 10E) and OCI/AML2 (FIG. 10H) cells exhibited relatively low amount of intracellular fluorescence after 2-hour incubation at 37° C. In contrast, no obvious internalization was observed with HCT-116 cells despite high surfacing binding of MAb52-29.1mAb-FITC (FIG. 10G).

MAb52-25.1mAb-FITC was partially internalized by A375 and BGC823 cells (FIGS. 10A and 10B, middle panels), whereas the efficiency was lower than MAb52-29.1mAb-FITC.

These findings implied that internalization efficiency of MAb52-29.1mAb-FITC or MAb52-25.1mAb-FITC triggered by CD166 binding varies in different cell lines. This variation may be influenced by intrinsic turnover rates of CD166 and PTMs such as glycosylation, phosphorylation, and ubiquitination. Efficient internalization of MAb52-29.1 mAb and MAb52-25.1mAb in multiple cancer types supporting its potential as an ADC candidate for multi-cancer treatment.

### Example 4

Molecular Cloning of the Light and Heavy Chain Genes and Expression of Chimeric Antibodies (cAbs)

### A. Cloning and Sequencing of the Light and Heavy Chain Genes by 5′-RACE

The cDNA sequences encoding the variable light (VL) and variable heavy (VH) regions of the MAb52-29.1 and MAb52-25.1 hybridoma were determined using 5′-Rapid Amplification of cDNA Ends (5′-RACE). Total RNA was extracted from the hybridoma cells using the Quick-RNA™ Miniprep Kit (Zymo Research). Reverse transcription and first-strand cDNA synthesis were performed using the SMARTer RACE 5′/3′ Kit (Takara Bio USA, San Jose, CA). The resulting cDNAs was then subjected to 5′-RACE PCR amplification using the following reverse primers to pair with the common forward primer, Universal Primer A Mix (UPM), provided by the kit.

Light chain reverse primer:
(SEQ ID NO: 24)
5′-CTGCTCACTGGATGGTGGGAAGATGG-3′
Heavy chain reverse primer:
(SEQ ID NO: 25)
5′-AGCTGGGAAGGTGTGCACAC-3′

The amplified PCR products were cloned into the pRACE vector (Takara) and transformed into Stellar™ E. coli competent cells (Takara) under ampicillin selection. Ten E. coli colonies containing VL and VH DNA fragments were randomly selected for plasmid purification using Hi-Speed Mini Plasmid Kits (IBI Scientific, Dubuque, Iowa). The purified plasmids were subjected to Sanger sequencing (Molecular Cloning Laboratories, South San Francisco, CA), and all sequences were analyzed through BLAST (Basic Local Alignment Search Tool) search against public databases, including NCBI IgBlast (https://www.ncbi.nlm.nih.gov/igblast/), the international ImMunoGeneTics (IMGT) Information System® (http://www.imgt.org/IMGT_vquest/), and Uniprot (https://www.uniprot.org/blast), for the novelty of the V domains.

Consensus cDNA sequences of VL and VH of MAb52-29.1 mAb are shown below as SEQ ID NO: 1 and SEQ ID NO: 3, respectively. The encoded amino acid sequences are shown in the SUMMARY section as SEQ ID NO: 2 and SEQ ID NO: 4, respectively.

(SEQ ID NO: 1)
GACATCCAGATGACACAATCTTCATCCTCCTTTTCTGTATCTCTAGGAGACAGAGTCACCATTA
CTTGCAAGGCAAGTGAGGACATATATAATCGATTAGCCTGGTATCAGCAGAAACCAGGAAATGC
TCCTAGGCTCTTAATATCAGGTGTAAGCAGTTTGGAAGCTGGAATTCCTTCAAGATTCAGTGGC
AGTGGATCTGGAAAGGATTACACTCTCAGCATTTCCAGTCTTCAGACTGAAGATGTTGCTGCTT
ATTACTGTCAACAGTATTGGAGTTCTCCGTACACGTTCGGAGGGGGGACCAAACTGGAAATAAA
A
(SEQ ID NO: 3)
GAAGTGAAGCTGGTGGAGTCTGGGGGAGGCTTAGTGCAGCCTGGAGGGTCCCTGAAACTCTCCT
GTGCAGCCTCTGGATTCACTTTCAGGACCGCTTTTATGTCTTGGATTCGCCAAACTCCAGAGAA
GAGGCTGGAATTGGTCGCAGAAATTAGTGATAGTGGTGGCAGCACCTACTTTTCAGACACTGTA
AAGGGCCGATTTACCATCTCCAGAGACAATGCCAAGAACAGTCTGTACCTGCAAATGAGTAGTC
TGAAGTCTGAGGACACGGCCATGTATTACTGTGCAAGACGGGGACAGCTCGGACTACGGGGATT
CTTTGACTGCTGGGGCCAAGGCACCACTCTCACAGTCTCCTCA

Consensus cDNA sequences of VL and VH of MAb52-25.1 mAb are shown below as SEQ ID NO: 11 and SEQ ID NO: 13, respectively. The encoded amino acid sequences are shown in the SUMMARY section as SEQ ID NOs: 12, 14, respectively.

(SEQ ID NO: 11)
GATATCCAGATGACACAGACTACATCCTCCCTGTCTGCCTCTCTGGGAGACAGAGTCACCATCG
ATTGCAGGGCGAGTCAGGACATCAACAATTATTTAAACTGGTATCAACAGAAACCAGATGGAAC
TGTTAAACTCCTGATCCACTACACATCAAGATTACACTCAGGAGTCCCATCAAGGTTCAGAGGC
AGTGGGTCTGGAACAGATTTTTCTCTCACCATTACCAACCTGGAACAAGAAGATGTTGCCACTT
ACTTTTGTCAACAGGGTAATACGATGTGGACGTTCGGTGGAGGCACCAAGTTGGATATCAAA
(SEQ ID NO: 13)
GAAGTGAAACTGGTGGAGTCTGGGGGAGGCTTAGTGGAGCCTGGAACGTCTCTGAAACTCTCCT
GTGCAGCCTCTGAATTCACTTTCAGTAATTATGGAATGTCTTGGGTTCGCCAGACTTCAGACAA
GAGGCTGGAGTGGGTCGCATCCATTAGTAGTGGTAATGGGGTTTATTATTCAGACAATGTAAAG
GGCCGATTCACCATCTCCAGAGAGAATGCCAAGAACACCCTGTATTTGCAAATGAGTAGTCTGA
AGTCTGAAGACACGGCCTTATATTACTGTGCAAGAGTTGATGGTAGTTATGTATATTTCGATGT
CTGGGGCTCAGGGACCACGGTCACCGTCTCCTCA

B. Construction of Expression Plasmid for MAb52-29.1cAbm2

A free cysteine residue (Cys or C) was found in the CDR3 of MAb52-29.1 mAb heavy chain at position 102 (Kabat numbering system). To avoid structural instability and aggregation of recombinantly expressed antibodies, Cys102 was substituted with serine (Ser or S) by site-directed mutagenesis using primer pairs shown as SEQ ID NO: 35 and 36 below. The encoded CDR3-H3 is referred as CDR-H3-C102S (SEQ ID NO: 37) below with the C102S point mutation shown in bold.

29C102SF:
(SEQ ID NO: 35)
5′-CGGGGATTCTTTGACTCCTGGGGCCAAGGCACC-3′
29C102SR:
(SEQ ID NO: 36)
5′-GGTGCCTTGGCCCCAGGAGTCAAAGAATCCCCG-3′
CDR-H3-C102S:
(SEQ ID NO: 37)
RGQLGLRGFFDS

The VL and VH genes of MAb52-29.1 mAb and MAb52-25.1 mAb were then amplified via PCR using Q5® High-Fidelity DNA Polymerase (New England Biolabs) from the sequencing plasmids using the cloning primer pairs shown as SEQ ID NO: 38 to 45 below.

29VL-F:
(SEQ ID NO: 38)
5′-AGCACTAGTGCCGCCACCATGAAGTTTCCTTCTCAAC-3′
29VL-R:
(SEQ ID NO: 39)
5′-GGGCGCCGCTACAGTCCGTTTTATTTCCAGTTTGG-3′
29VH-F:
(SEQ ID NO: 40)
5′-AGCTCTAGAGCCGCCACCATGAATTTCGGGCTCAG-3′
29VH-R:
(SEQ ID NO: 41)
5′-GGTGCTAGCTGAGGAGACTGTGAGAGTGGTGC-3′
25VL-F:
(SEQ ID NO: 42)
5′-AGAACTAGTGCCGCCACCATGTCCTCTGCTCAGTTCC-3′
25VL-R:
(SEQ ID NO: 43)
5′-CTGGGCGCCGCTACAGTCCGTTTGATATCCAACTTGG-3′
25VH-F:
(SEQ ID NO: 44)
5′-AGCTCTAGAGCCGCCACCATGAACTTCGGGCTCAGC-3′
25VH-R:
(SEQ ID NO: 45)
5′-GTGCTAGCTGAGGAGACGGTGACCGTGGTCCCTGAG-3′

The PCR products were gel-purified using the Monarch DNA Gel Extraction Kit (New England Biolabs) and cloned in-frame into the pCMV-hIgG1K-m2 vector with NEBuilder® HiFi DNA Assembly Cloning Kit (New England Biolabs), following the manufacturer's protocol. The pCMV-hIgG1K-m2 vector encodes the human kappa light chain constant region (CL) and the human IgG1 heavy chain constant region (CH1-CH3), under the control of the murine CMV promoter and the human CMV IE1 promoter, respectively. Additionally, each hIgG1 constant region contains two cysteine point mutations, enabling rapid screening of ADC candidates via site-specific conjugation to various payloads and linkers. Upon in-frame fusion with the murine MAb52-29.1 or MAb52-25.1 VL and VH genes, the resulting pCMV-52-29.1cAbm2 and pCMV-52-25.1cAbm2 plasmids facilitate the expression of human-murine cAbs incorporating four free Cys residues, named MAb52-29.1cAbm2 and MAb52-25.1cAbm2, respectively.

C. Expression of MAb52-29.1cAbm2 and MAb52-25.1cAbm2

Following confirmation by Sanger sequencing, pCMV-52-29.1cAbm2 and pCMV-52-25.1cAbm2 were individually electroporated into Dux-1S-HD cells, a CHO DG44-derived cell line capable of high-density growth in serum-free medium (SFM), such as CD DG44 medium (Thermo Fisher Scientific). The cells were then plated at a density of 2×10⁵ cells/well in selection medium in 96-well tissue culture microplates. After 2-3 weeks of incubation in a CO₂ incubator, stable cell lines secreting MAb52-29.1cAbm2 or MAb52-25.1cAbm2 into the culture medium were screened by ELISA to assess antibody expression and binding to rhCD166-ECD-His. The selected cell line was subsequently subcloned by limited dilution, adapted to SFM, and expanded for large-scale production of MAb52-29.1cAbm2 or MAb52-25.1cAbm2. MAb52-29.1cAbm2 and MAb52-25.1cAbm2 purified from the culture supernatants by Protein-A affinity chromatography was analyzed by SDS-PAGE to ensure the integrity and the purity.

### Example 5

Characterization of MAb52-29.1 and MAb52-25.1 cAbs for Binding to rhCD166-ECD-his Protein and Cancer Cell Lines
A. Competitive ELISA Assay of MAb52-29.1 mAb and MAb52-29.1cAbm2 in Antigen Binding

As expected, both MAb52-29.1cAbm2 and MAb52-25.1cAbm2 bound to rhCD166-ECD-His like their murine parental mAbs in ELISA assays (data not shown). A competitive ELISA assay was performed to determine whether MAb52-29.1cAbm2 retains the ability to recognize the same epitope on rhCD166 as MAb52-29.1 mAb. In this assay, unconjugated MAb52-29.1 mAb, MAb52-29.1cAbm2, or an isotype control cAbm2 with human kappa CL and IgG1 CH1-CH3 were serially diluted in three folds, mixed with 1:4,000 diluted MAb52-29.1mAb-HRP conjugated as shown in Example 2A, and incubated with immobilized rhCD166-ECD-His for one hour. After thorough washing, TMB substrate was added to detect binding signals between MAb52-29.1 mAb-HRP and the antigen.

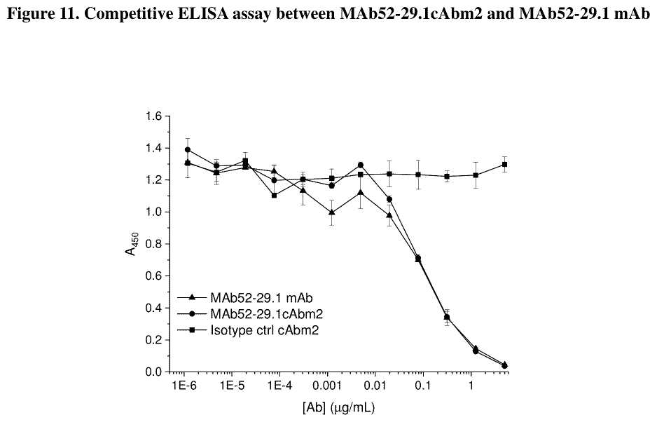

As shown in FIG. 11, the isotype control cAbm2, which targets an irrelevant antigen, served as the baseline for MAb52-29.1mAb-HRP and rhCD166-ECD-His binding. Both MAb52-29.1 mAb and MAb52-29.1cAbm2 exhibited a dose-dependent reduction in binding signals with nearly overlapping curves, indicating that they recognize overlapping or identical epitopes on CD166 and possess comparable apparent affinities. Therefore, substituting the murine antibody constant regions with human heavy chain constant regions containing Cys point mutations and the C102S mutation in CDR-H3 did not affect epitope recognition.

B. Binding Kinetics and Affinity Between MAb52-29.1cAbm2 and rhCD166-ECD-his

The interaction between MAb52-29.1cAbm2 and rhCD166-ECD-His was analyzed using the Octet® QKe System equipped with Anti-Human IgG Fc Capture (AHC) Biosensors (Sartorius) following the protocol described in Example 2D. One hundred nanomolar (100 nM) MAb52-29.1cAbm2 was immobilized onto the AHC biosensors for 10 min followed by a 5-min wash, a 15-min association phase with rhCD166-ECD-His, and a 30-min dissociation phase. The assay was repeated multiple times with rhCD166-ECD-His concentrations ranging from 200 nM to 6.25 nM, using 2-fold serial dilutions. A 1:1 binding model was applied to fit the binding curves.

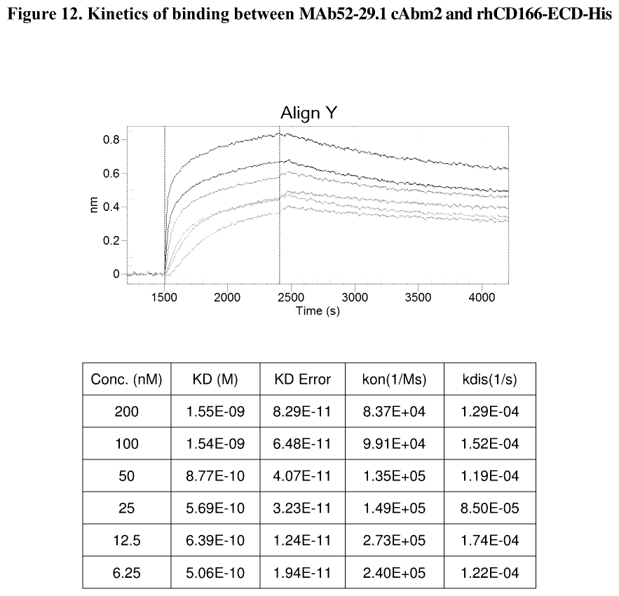

As shown in FIG. 12, MAb52-29.1cAbm2 exhibited a fast association rate and a slow dissociation rate, with a calculated K_{D} of approximately 0.95×10⁻⁹ M that was similar to the murine mAb counterpart (1.12×10⁻⁹ M; FIG. 7). This result further demonstrated that the point mutations in CDR-H3 and the constant regions did not interfere with the binding affinity of MAb52-29.1cAbm2.

C. FACS Analysis of cAbs Binding to Various Cancer Cell Lines

FACS analysis was conducted to assess the interaction between MAb52-29.1cAbm2 or MAb52-25.1cAbm2 with various cancer cell lines, following the protocol outlined in Example 1. The only modification was the replacement of the anti-mouse IgG secondary antibody with Fluorescein AffiniPure™ Goat Anti-Human IgG, Fcγ fragment specific, to detect surface-bound cAbm2. An isotype control cAbm2, consisting of human IgG1 CH1-CH3 and human Kappa CL, served as the negative control.

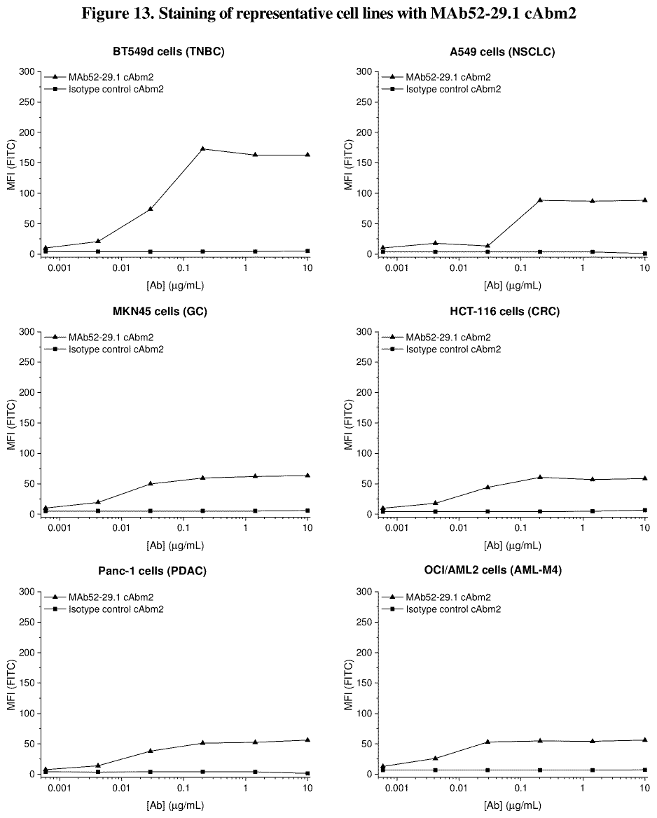

Cell staining of six representative human cancer cell lines confirmed a dose-dependent binding pattern for MAb52-29.1cAbm2 (FIG. 13), comparable to MAb52-29.1 mAb (see FIGS. 1A and 1B). MAb52-25.1cAbm2 also bound to two tested cancer cell lines as well as HEK293FT cells, similar to its mAb counterpart (data not shown). These results suggest that replacement of murine constant regions with human constant regions, including point mutations, did not compromise the binding specificity of the chimeric antibodies.

### Example 6

Preparation and Characterization of ADCs Derived from MAb52-29.1cAbm2 and MAb52-25.1cAbm2
A. Site-Specific Conjugation of MAb52-29.1cAbm2 and MAb52-25.1cAbm2 with Vc-MMAE

MAb52-29.1 mAb and MAb52-25.1 mAb demonstrated strong cancer-specificity and internalization capability in multiple cancer cell lines, making them a promising candidate for ADC therapy. To further explore their potential as ADC candidates, MAb52-29.1cAbm2, MAb52-25.1cAbm2, and the non-cell binding isotype control cAbm2, were engineered with two Cys point mutations in each IgG1 heavy chain constant region for site-specific conjugation using the maleimide-valine-citrulline-PAB-MMAE (VcMMAE) linker-payload reagent (BroadPharma, San Diego, CA). VcMMAE consists of a lysosomal-cleavable dipeptide linker (Vc), a para-aminobenzyloxycarbonyl (PABC) spacer, an anti-tubulin toxin MMAE, and a thiol-reactive maleimidocaproyl (MC) group that specifically reacts with the free thiol group in cysteines. VcMMAE has been demonstrated to be an effective payload-linker in ADC in treating many types of cancer. It is a key component in FDA-approved ADCs, including Brentuximab vedotin (Adcetris®), Polatuzumab vedotin (Polivy®), Enfortumab vedotin (Padcev®), and Tisotumab vedotin (Tivdak®) (Gogia et al. Cancers, 2023; Liu et al. Mol Cancer 2024). Consequently, we employed VcMMAE in our ADC platform to quickly screen mAbs as ADC candidates.

Site-specific conjugation was performed following the method described by Junutula et al. (Nat Biotechnol. 26:925-932, 2008). Briefly, MAb52-29.1cAbm2, MAb52-25.1cAbm2, and the non-cell binding isotype control cAbm2 antibodies were reduced with 1 mM dithiothreitol (DTT) to remove cysteine or glutathione adducts from the culture medium. The reduced antibodies were then refolded into their native structures in the presence of 0.4 mM dehydroascorbic acid (DHAA; MilliporeSigma), and the unfolded light and heavy chains were removed using an Amicon® Ultra Centrifugal Filter (50 kDa MWCO; MilliporeSigma). For conjugation, VcMMAE was mixed with each refolded antibody at a 10:1 molar ratio with gentle agitation at RT for one hour, followed by size exclusion chromatography to remove unbound VcMMAE and potential aggregates. This site-specific conjugation protocol results in ADCs with an average drug-to-antibody ratio (DAR) of approximately 4 (DAR4).

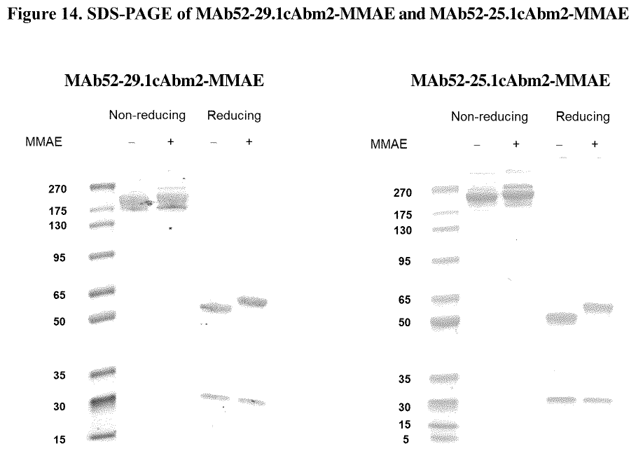

The concentration of each cAbm2-MMAE was measured using NanoDrop™ 2000 at 280 nm and confirmed with the Pierce™ Bradford Protein Assay Kit (Thermo Fisher Scientific). SDS-PAGE analysis under reducing conditions revealed distinct light and heavy chain bands, with no visible aggregation or degradation (FIG. 14). As expected, the MMAE-conjugated heavy chain bands migrated higher than the unconjugated heavy chain bands, indicating successful conjugation. Non-reducing SDS-PAGE showed a minor, but acceptable, fraction of aggregates.

B. In Vitro Inhibitory Effects of MAb52-29.1cAbm2-MMAE and MAb52-25.1cAbm2-MMAE on Cancer Cell Proliferation

The effect of MAb52-29.1cAbm2-MMAE and MAb52-25.1cAbm2-MMAE on cancer cell proliferation was evaluated using cancer cell line-based cytotoxic assays as previously described (see U.S. Pat. No. 11,939,391B2). Briefly, each cancer cell line cultured in DMEM with 10% (v/v) FBS was seeded at 3-5×10³ cells/well, depending on cell type, in a 96-well tissue culture microplate. Cells in triplicate wells were treated with MAb52-29.1cAbm2-MMAE, MAb52-25.1cAbm2-MMAE, or isotype control cAbm2-MMAE at serial dilutions with concentrations starting from 3 to 15 μg/mL (20~100 nM). After four days of incubation at 37° C. under a 5% CO₂ humidified atmosphere, cell viability was assessed by adding 1/10 volume of the Cell Counting Kit-8 (CCK-8) solution (Dojindo Molecular Technologies, Rockville, MD) and incubating for four hours at 37° C., followed by measuring the absorbance at 450 nm (A450).

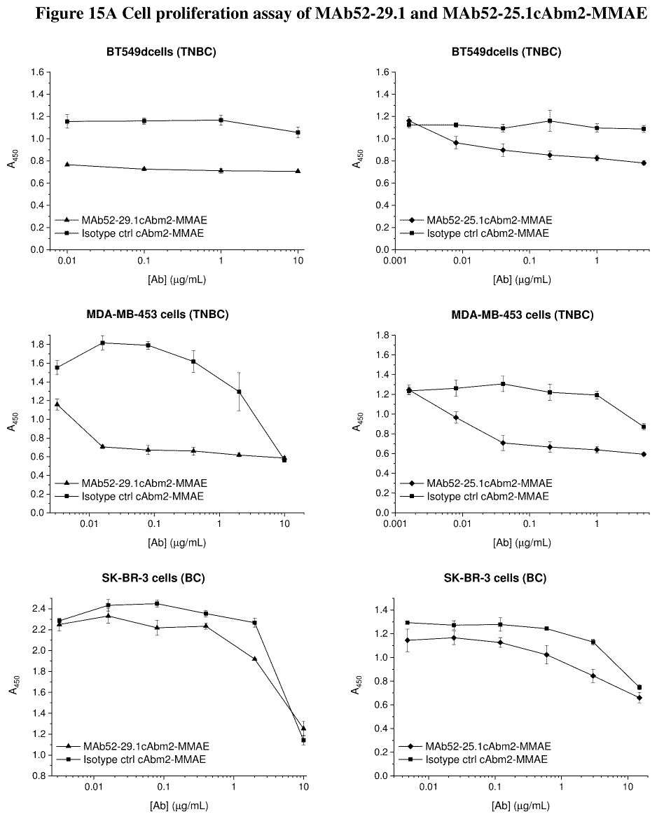

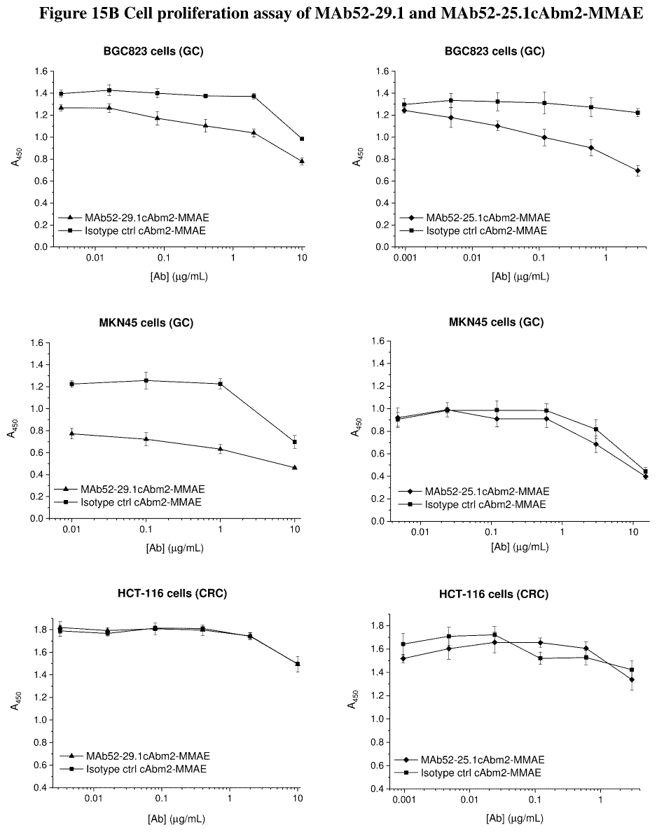

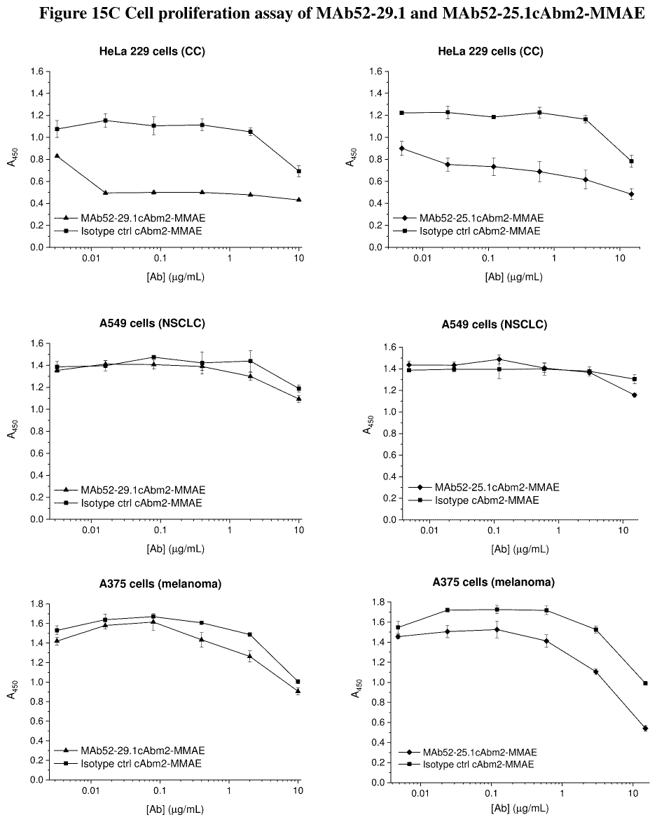

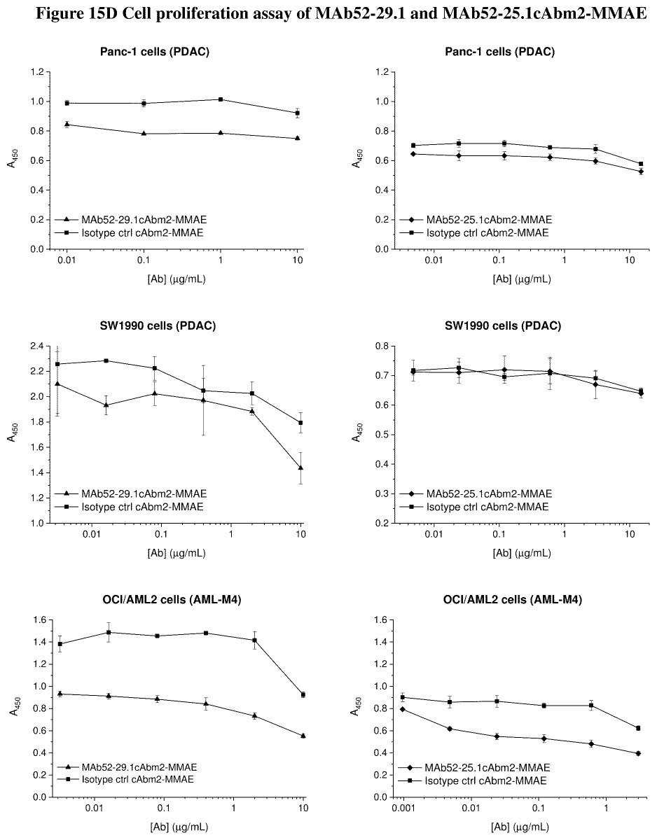

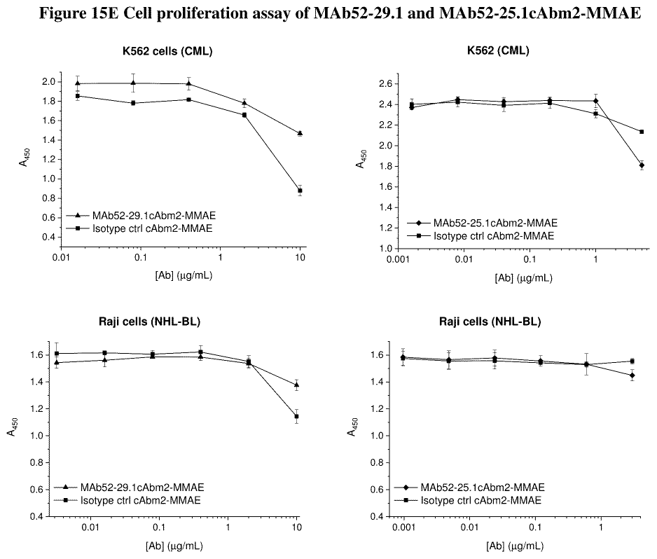

Exposure of the representative TNBC, GC, CC, and AML cell lines to MAb52-29.1cAbm2-MMAE or MAb52-25.1cAbm2-MMAE led to significant, and dose-related reductions in cell proliferation (FIG. 15). In contrast, SK-BR-3 (BC), HCT-116 (CRC), A549 (NSCLC), A375 (melanoma) and most PDAC cell lines showed weak or no response to either ADCs. These findings suggest that the anti-proliferative effect of MAb52-29.1cAbm2-MMAE and MAb52-25.1cAbm2-MMAE is restricted to certain types of cancer that is not correlation with the abundance of the epitopes, but rather proportional to the internalization efficiency of the anti-CD166 and CD166 complexes.

### Example 7

In Vivo Tumor Inhibition Study of MAb52-29.1cAbm2-MMAE on Cancer CDX Mouse Models

Both MAb52-25.1 mAb and MAb52-29.1 mAb bound to human CD166 with different conformational epitopes, whereas MAb52-29.1 mAb exhibited slightly higher cellular binding affinity and internalization efficiency than MAb52-25.1 mAb. Consequently, the MAb52-29.1 mAb-derived ADC was further investigated for its anti-tumor activities in cancer cell-derived xenograft (CDX) mouse models.

Six CDX mouse models, including BGC823, MKN45, BT549d, A549, A375, and HCT-116, were utilized to evaluate the antitumor effects of a single-dose treatment with MAb52-29.1cAbm2-MMAE (DAR~4). For each model, 2×10⁶ to 5×10⁶ cancer cells, suspended in 0.1 ml of PBS, were subcutaneously (s.c.) inoculated into the flanks of 6-week-old male athymic nude mice (Charles River Laboratories, Wilmington, MA). Mice were randomized into five per treatment group when the majority of tumors reached 100 mm³ in size (Day 0).

Mice received intraperitoneal (i.p.) injections of either MAb52-29.1cAbm2-MMAE or isotype control cAbm2-MMAE (DAR~4) at doses of 4 mg/kg, 7 mg/kg, or 10 mg/kg. In some studies, a vehicle-only control group was treated with 0.1 ml of PBS to assess the potential off-target effect of the ADCs. Tumor size and body weight were monitored every three to four days over four to five weeks, depending on tumor aggressiveness. Tumor volumes were calculated using the formula: Volume=1/2×(width)²×length. Animals were euthanized if tumor volume exceeded 2,000 mm³ or if the tumor diameter surpassed 20 mm.

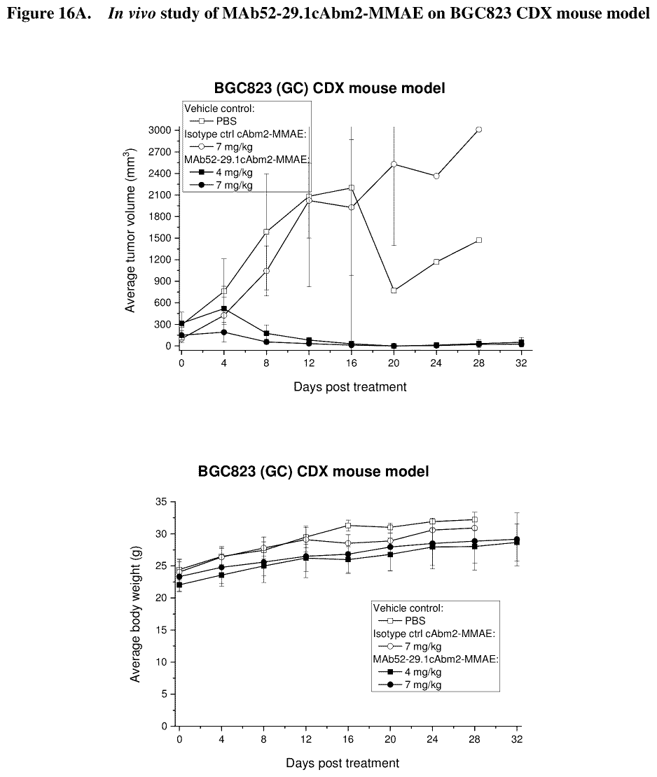

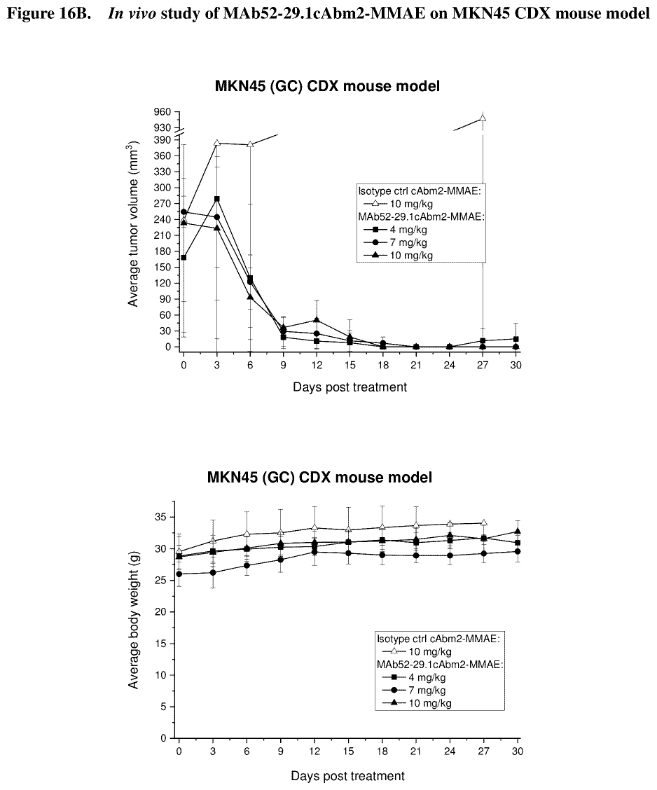

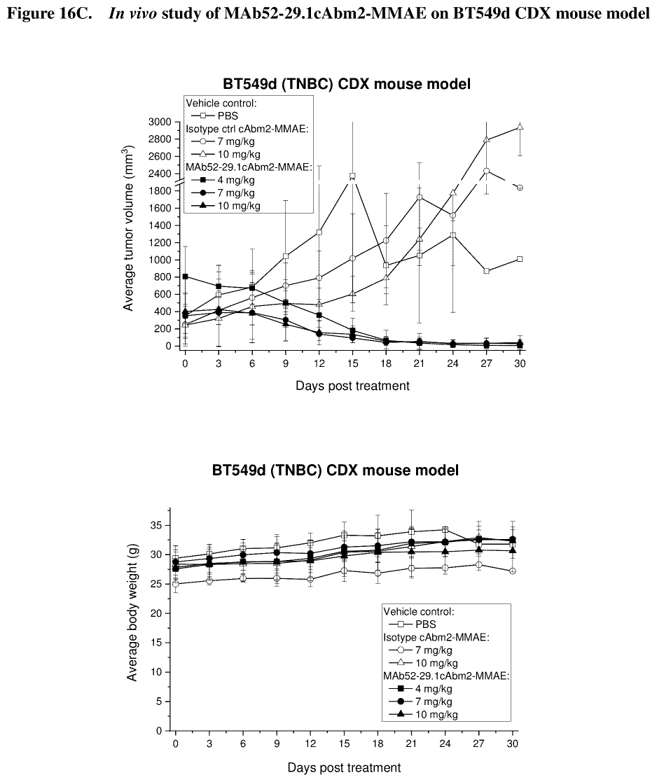

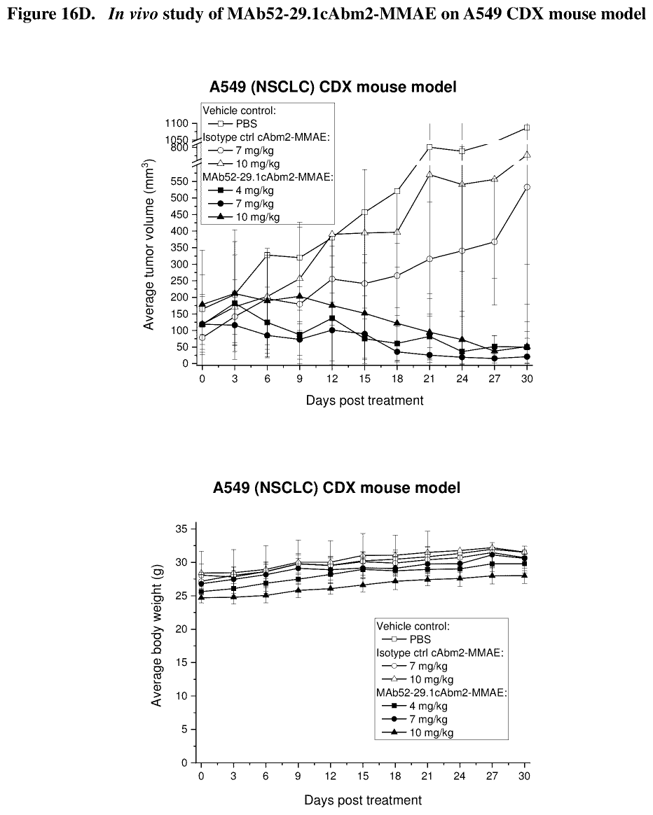

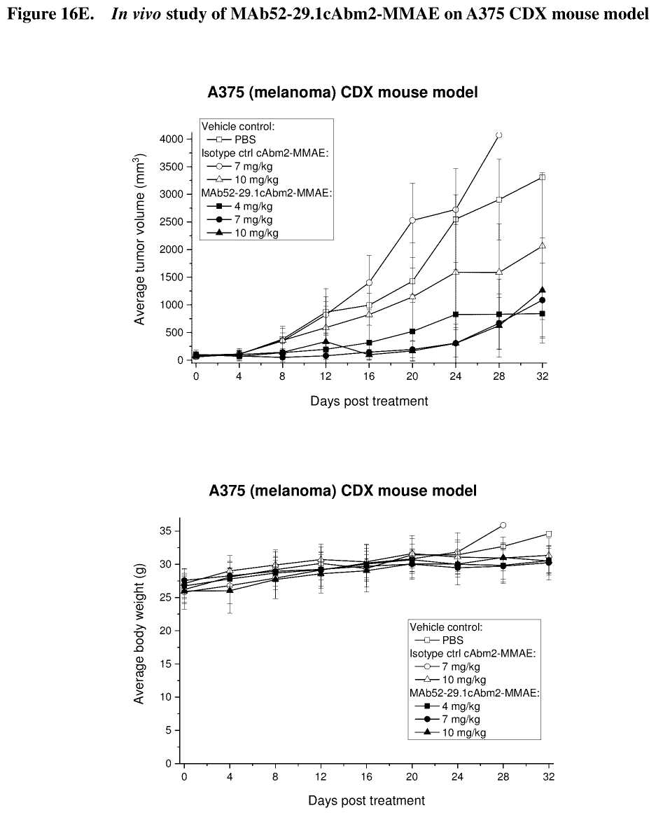

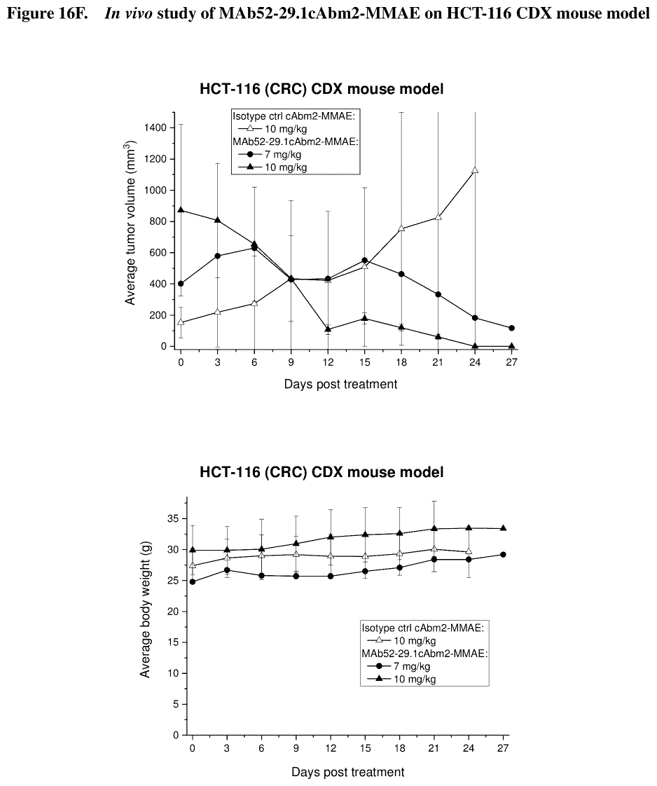

FIGS. 16A to 16F illustrate the average tumor volume and body weight in each treatment group. Compared to the vehicle-treated and isotype control cAbm2-MMAE-treated groups, MAb52-29.1cAbm2-MMAE significantly reduced tumor volumes in the BGC823, MKN45, BT549d, and A549 CDX models across all three tested doses (FIGS. 16A to 16D). In these models, tumor nodules halted growth within six to eight days post-treatment, with most tumors continuing to regress until became undetectable, suggesting a strong bystander effect of the MAb52-29.1 ADC.

In contrast, MAb52-29.1cAbm2-MMAE was less effective against A375 tumors as it reduced tumor growth rate but failed to achieve full suppression (FIG. 16E), indicating that multiple-dose treatment may be necessary for complete tumor eradication. Preliminary results from the HCT-116 tumor model showed encouraging tumor regression in cases where large tumors had reached sizes of 1400 to 1800 mm³ prior to treatment recessed upon MAb52-29.1cAbm2-MMAE treatment (FIG. 16F). However, due to the small sample size and a large variation of tumor size, a definitive conclusion could not be drawn.

Nevertheless, the anti-tumor effect of a single-dose MAb52-29.1cAbm2-MMAE administration persisted for more than three to four weeks across multiple models. It is anticipated that multiple-dose regimens could achieve complete tumor control in these CDX models.

In the BT549d, A549, and A375 CDX models, the isotype control cAbm2-MMAE exhibited a slightly reduced tumor growth rate compared to the vehicle-treated group (FIGS. 16C, 16D, and 16E), suggesting a minor non-specific effect, potentially due to the instability of the cleavable Val-Cit-PAB linker in vivo. While this difference was not significant, it highlights the need for a more stable linker in future ADC development.

As observed in the in vitro assays, the varying responses of different tumor models to MAb52-29.1cAbm2-MMAE may be influenced by several factors, including differences in surface exposure of the binding epitope on CD166, the internalization efficiency of the ADC-CD166 complex, and cellular sensitivity to MMAE. Additionally, in in vivo studies, ADC penetration through the tumor microenvironment (TME) could further impact therapeutic efficacy.

No adverse effects, such as weight loss, fever, lethargy, vomiting, diarrhea, bleeding, or loss of appetite, were observed in any of the MMAE-conjugated antibody-treated groups.

### Example 8

### Humanization of MAb52-29.1 mAb and Verification of Target Binding for MAb52-29.1HuAbm2s

### A. Engineering MAb52-29.1 HuAbs Via CDR Grafting and Strategic Back-Mutations

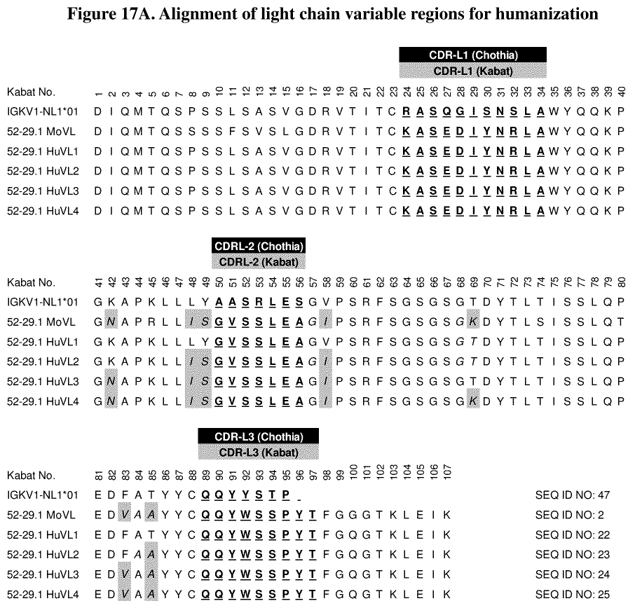

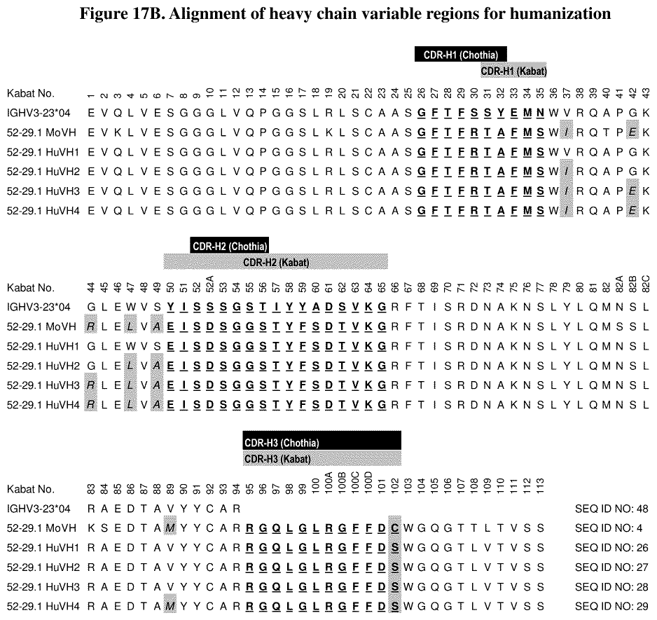

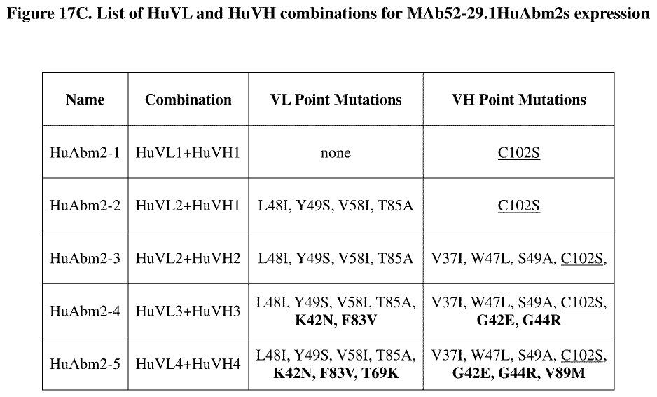

The VL (SEQ ID NO: 2) and VH (SEQ ID NO: 4) regions of murine MAb52-29.1 mAb were compared against human antibody germline sequences available in public databases. The optimal human germline sequences selected as the templates for humanization are shown in FIGS. 17A and 17B. Specifically, the VL frameworks (FRs) of human IGKV1-NL1*01 (SEQ ID NO: 46) and the VH FRs from human IGHV3-23*04 (SEQ ID NO: 47) were chosen to replace the corresponding murine FRs of MAb52-29.1 mAb. The joint (J) region sequences selected were from human IGKJ2 (YTFGQGTKLEIK, SEQ ID NO: 48) and IGHJ5 (DSWGQGTLVTVSS, SEQ ID NO: 49) sequences. The sequence identities between murine and human FRs range from 73.3% to 82.6% for VL and 57.1% to 90% for VH. The HuVL1 (SEQ ID NO: 21) and HuVH1 (SEQ ID NO: 25) sequences were designed using CDR grafting, incorporating murine CDRs into human FR sequences. In contrast, HuVL2, HuVL3, HuVL4 (SEQ ID NOs: 22, 23, 24) and HuVH2, HuVH3, HuVH4 (SEQ ID NOs: 26, 27, 28) include back mutations to preserve epitope binding and structural integrity. FIG. 17C shows the specific mutations in these HuVLs and HuVHs, along with their combinations to generate five humanized MAb52-29.1 variants.

The cDNA fragments encoding these HuVL and HuVH sequences were synthesized and annealed respectively with the human light and heavy chain constant regions encoded by the pCMV-hIgG1K-m2 expression vector following the protocol described in Example 4B. The expression plasmids also contained two free cysteine point mutations in each of the Fc region to allow site-specific conjugation via disulfide bonds. Sanger sequencing confirmed the antibody coding regions in the plasmids, which were then transfected into HEK293FT cells (Thermo Fisher Scientific) using FectoPro™ Transfection Reagent (PolyPlus Illkirch, France) according to the manufacturer's protocol. Cell cultures were maintained in 30 mL of SFM containing DMEM with 1.0% (w/v) BSA. Five days post-transfection, the five humanized antibodies, designated as MAb52-29.1HuAbm2-1 to -5, secreted in the culture supernatants were quantified using a capture ELISA assay immobilized with AffiniPure™ Goat Anti-Human IgG, Fcγ Fragment Specific polyclonal antibody (Jackson ImmunoResearch laboratories), and assessed for antigen binding by ELISA.

B. Binding of Humanized MAb52-29.1 with rhCD166-ECD-his Assessed by ELISA

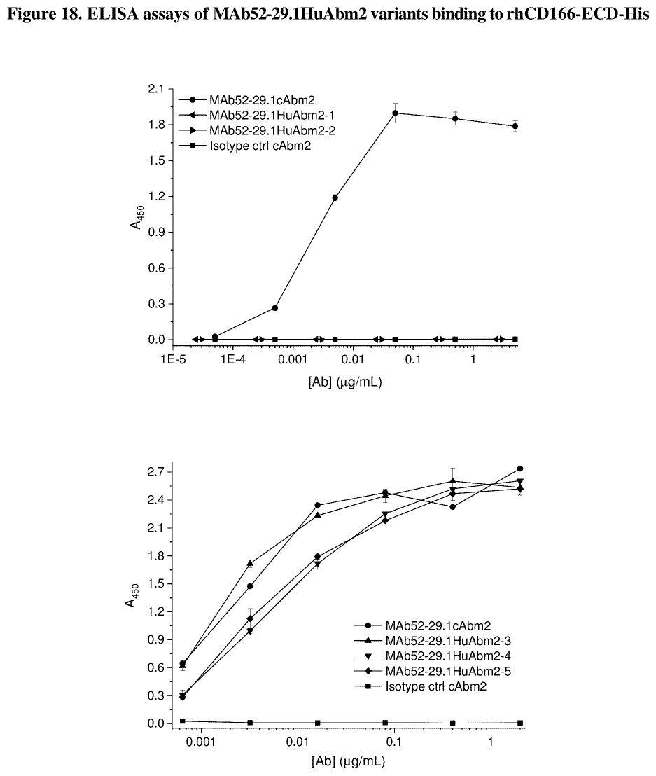

The culture supernatants containing different MAb52-29.1HuAbm2 variants were assessed for binding to immobilized rhCD166-ECD-His using an ELISA assay. The results showed that HuAbm2-1, which lacked FR back mutations, and HuAbm2-2, which contained four FR back mutations in VL, failed to bind rhCD166-ECD-His (FIG. 18, upper panel). In contrast, HuAbm2-3, HuAbm2-4, and HuAbm2-5 restored binding capacity in a dose-dependent manner (FIG. 18, lower panel), indicating that the back mutations were essential for target recognition. However, HuAbm2-4 and HuAbm2-5 exhibited lower binding affinity compared to HuAbm2-3, which displayed a binding curve similar to MAb52-29.1cAbm2, suggesting that additional back mutations impaired function. Based on these findings, the humanized variant comprising HuVL2 and HuVH2, with four back mutations in both VL and VH, was designated as MAb52-29.1HuAbm2 and selected for cell line generation and further evaluations.

C. Binding Kinetics and Affinity Between MAb52-29.1HuAbm2 and rhCD166-ECD-his

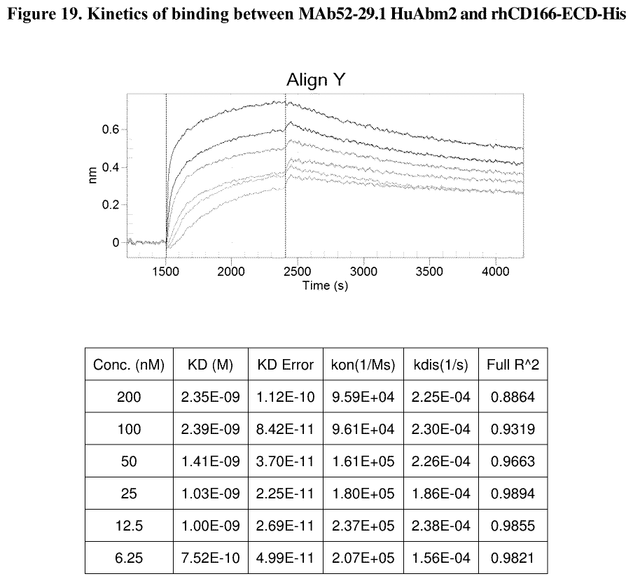

The binding kinetics and affinity between purified MAb52-29.1HuAbm2 and rhCD166-ECD-His were determined using the same protocol as MAb52-29.1cAbm2. MAb52-29.1HuAbm2 demonstrated an average binding affinity constant (K_{D}) of 1.56×10-9 M or 1.56 nM (FIG. 19), which is close to MAb52-29.1 mAb (1.12×10⁻⁹ M) and MAb52-29.1cAbm2 (0.95×10⁻⁹ M). These results indicate that replacing the murine FR sequences with the selected human FRs, along with the designed back mutations, did not compromise the antibody's binding affinity to its target.

### D. Binding and Internalization of MAb52-29.1HuAbm2 by Various Cancer Cell Lines

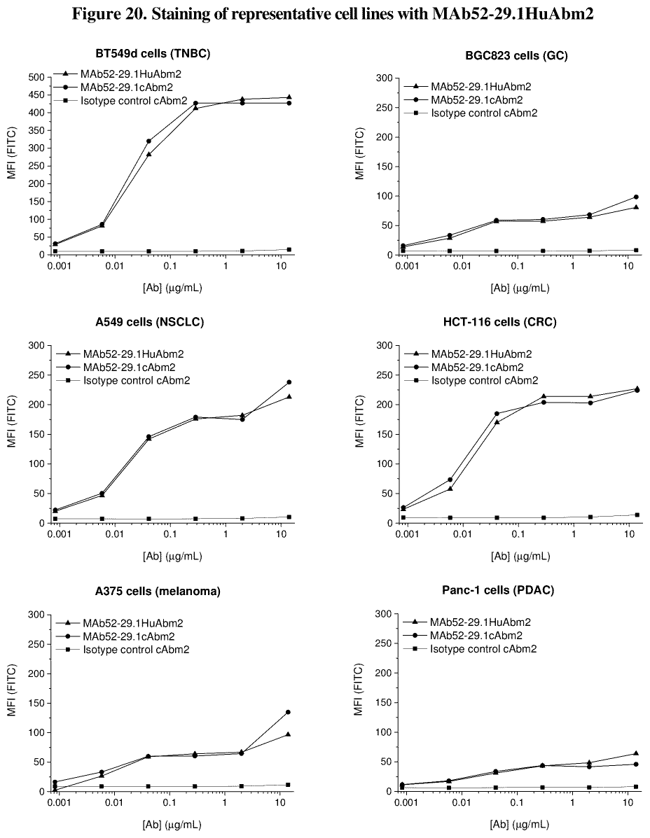

Cell staining of six human cancer cell lines, representing different types of cancer, confirmed a dose-dependent binding pattern for MAb52-29.1HuAbm2, comparable to MAb52-29.1cAbm2 (FIG. 20). The relative MFI values are consistent with those of MAb52-29.1 mAb in FACS assays (FIG. 1A).

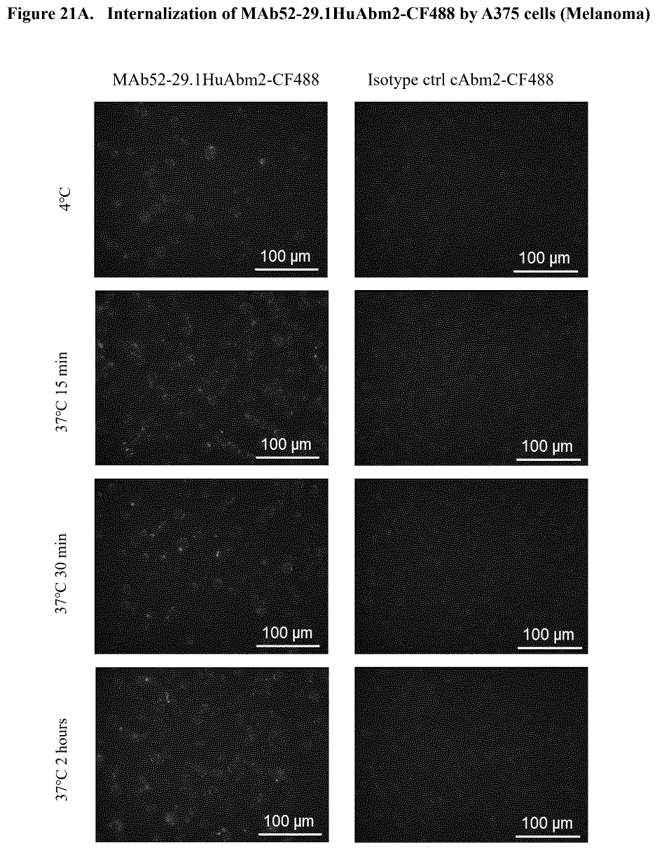

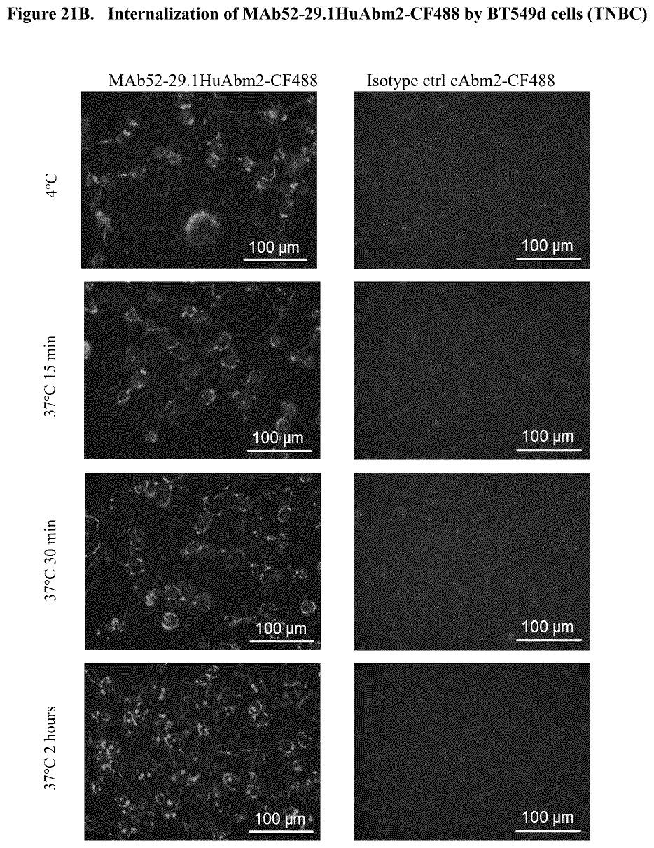

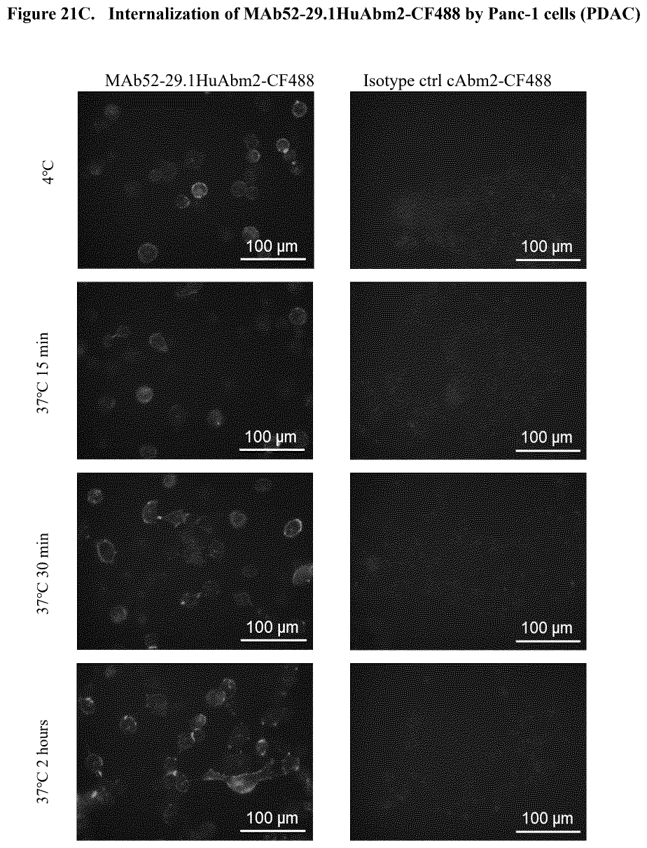

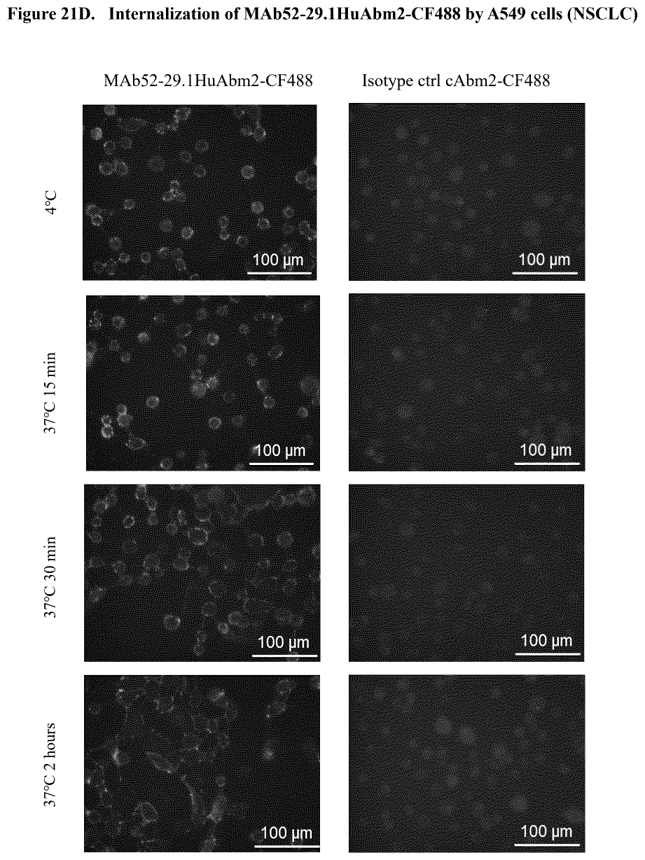

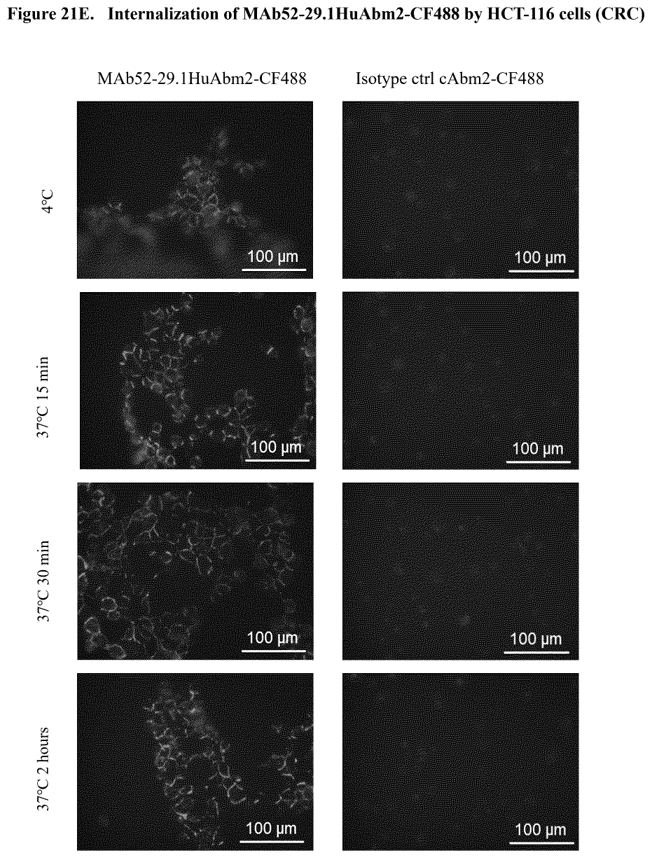

Using CF488-labeled MAb52-29.1HuAbm2 and an isotype control cAbm2, conjugated with the Mix-n-Stain Antibody Labeling Kit (Biotium, Fremont, CA), an internalization assay was conducted following the protocol described in Example 3B. MAb52-29.1mAb-CF488 was efficiently internalized by A375, BT549d, and Panc-1 cells (FIGS. 21A to 21C), whereas A549 and HCT-116 cells displayed limited intracellular fluorescence despite high surface binding signals (FIGS. 21D and 21E).

These findings indicate that humanization preserved the internalization capability of MAb52-29.1 mAb, further supporting its potential for ADC development in clinical cancer therapy.

### Example 9

### Preparation and Characterization of MAb52-29.1HuAbm2 ADC

A. Site-Specific Conjugation of MAb52-29.1HuAbm2 with Vc-MMAE

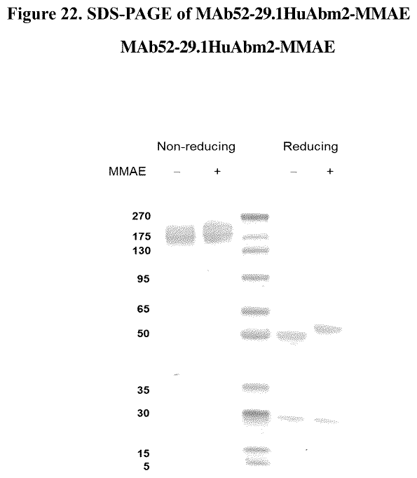

To evaluate whether the ADC derived from MAb52-29.1HuAbm2 has anti-cancer activity like MAb52-29.1cAbm2-MMAE, VcMMAE was used for site-specific conjugation following the procedure described in Example 6. MMAE was conjugated to four free cysteine residues within the heavy chain constant regions of MAb52-29.1HuAbm2, generating an ADC with approximately DAR4. SDS-PAGE analysis confirmed successful conjugation, as indicated by the apparent molecular weight shift of both the non-reduced MAb52-29.1HuAbm2-MMAE and the reduced heavy chain-MMAE (FIG. 22).

B. Binding of MMAE-Conjugated MAb52-29.1HuAbm2 and MAb52-29.1cAbm2 with Cancer Cell Lines

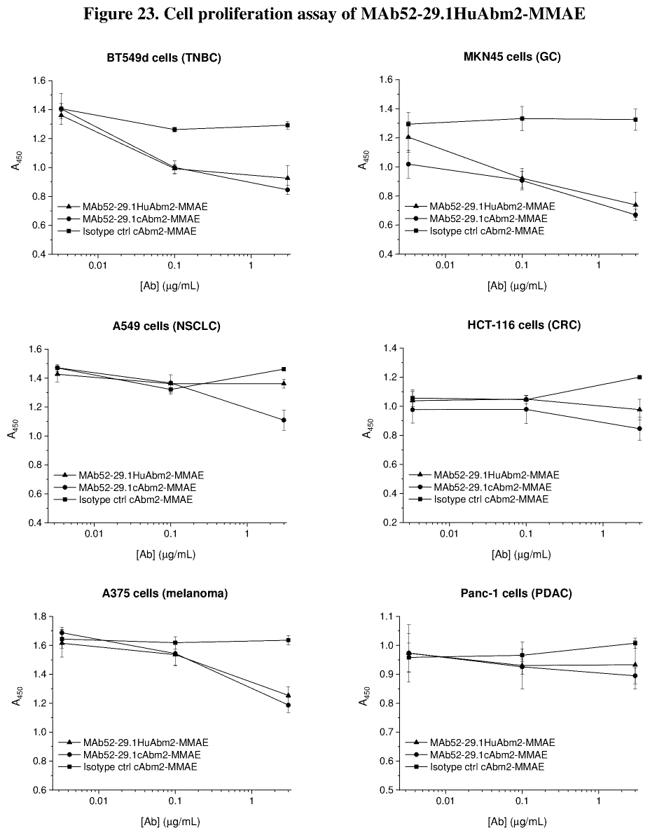

In FACS analyses, both humanized and chimeric MAb52-29.1 antibodies, with or without MMAE-conjugation, exhibited similar binding properties to various cancer cell lines tested (FIG. 23). This suggests that MMAE-conjugation to the Fc regions of the antibodies does not interfere with the binding activity of either MAb52-29.1cAbm2 or MAb52-29.1HuAbm2. Since MAb52-29.1HuAbm2 recognized overlapped epitope like MAb52-29.1cAbm2, and could be internalized by various cancer cell lines, it is reasonable to assume that MAb52-29.1HuAbm2-MMAE retains the same biological activity as MAb52-29.1cAbm2-MMAE.

### C. In Vitro Inhibitory Effects of MAb52-29.1HuAbm2-MMAE on Cancer Cell Proliferation

An in vitro cell proliferation assay was conducted following the protocol described in Example 6. MAb52-29.1HuAbm2-MMAE and MAb52-29.1cAbm2-MMAE exhibited similarly potent growth inhibition in A375, BT549d and MKN45 cell lines (FIG. 23). In contrast, both ADCs showed minimal effects on A549, HCT-116, and Panc-1 cells, which might be attributed to poor internalization properties with A549 and HCT-116 cell lines, and insensitivity of Panc-1 to MMAE toxicity.

In summary, MAb52-29.1HuAbm2 retained the binding capability to human CD166 and various human cancer cell lines. The tested ADC, MAb52-29.1HuAbm2-MMAE, demonstrated sustained inhibitory activity on representative TNBC, GC, and melanoma cell lines in vitro. We expect improved affinity and potency after antibody maturation. MAb52-29.1HuAbm2 is likely to maintain its anti-cancer potency in vivo, making it a promising drug candidate for cancer therapy in humans.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “including”, “containing”, “having” and “have” are to be read as synonyms and expansively and without limitation. The methods and processes illustratively described herein suitably may be practiced in differing orders of steps, and that they are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

TABLE 1
Sequence Listing

| SEQ ID | | |
|---|---|---|
| NO: | Sequence | Note |
| 1 | GACATCCAGATGACACAATCTTCATCCTCCTTTTCTGTATCTCTA | MAb52-29.1 moVL |
| | GGAGACAGAGTCACCATTACTTGCAAGGCAAGTGAGGACATATAT | |
| | AATCGATTAGCCTGGTATCAGCAGAAACCAGGAAATGCTCCTAGG | |
| | CTCTTAATATCAGGTGTAAGCAGTTTGGAAGCTGGAATTCCTTCA | |
| | AGATTCAGTGGCAGTGGATCTGGAAAGGATTACACTCTCAGCATT | |
| | TCCAGTCTTCAGACTGAAGATGTTGCTGCTTATTACTGTCAACAG | |
| | TATTGGAGTTCTCCGTACACGTTCGGAGGGGGGACCAAACTGGAA | |
| | ATAAAA | |
| 2 | DIQMTQSSSSFSVSLGDRVTITCKASEDIYNRLAWYQQKPGNAPR | MAb52-29.1 moVL |
| | LLISGVSSLEAGIPSRFSGSGSGKDYTLSISSLQTEDVAAYYCQQ | |
| | YWSSPYTFGGGTKLEIK | |
| 3 | GAAGTGAAGCTGGTGGAGTCTGGGGGAGGCTTAGTGCAGCCTGGA | MAb52-29.1 moVH |
| | GGGTCCCTGAAACTCTCCTGTGCAGCCTCTGGATTCACTTTCAGG | |
| | ACCGCTTTTATGTCTTGGATTCGCCAAACTCCAGAGAAGAGGCTG | |
| | GAATTGGTCGCAGAAATTAGTGATAGTGGTGGCAGCACCTACTTT | |
| | TCAGACACTGTAAAGGGCCGATTTACCATCTCCAGAGACAATGCC | |
| | AAGAACAGTCTGTACCTGCAAATGAGTAGTCTGAAGTCTGAGGAC | |
| | ACGGCCATGTATTACTGTGCAAGACGGGGACAGCTCGGACTACGG | |
| | GGATTCTTTGACTGCTGGGGCCAAGGCACCACTCTCACAGTCTCC | |
| | TCA | |
| 4 | EVKLVESGGGLVQPGGSLKLSCAASGFTFRTAFMSWIRQTPEKRL | MAb52-29.1 moVH |
| | ELVAEISDSGGSTYFSDTVKGRFTISRDNAKNSLYLQMSSLKSED | |
| | TAMYYCARRGQLGLRGFFDCWGQGTTLTVSS | |
| 5 | EDIYNRLA | MAb52-29.1 CDR-L1 |
| 6 | GVSSLEA | MAb52-29.1 CDR-L2 |
| 7 | QQYWSSPYT | MAb52-29.1 CDR-L3 |
| 8 | GFTFRTAFMS | MAb52-29.1 CDR-H1 |
| 9 | EISDSGGSTYFSDTVKG | MAb52-29.1 CDR-H2 |
| 10 | RGQLGLRGFFDC | MAb52-29.1 CDR-H3 |
| 11 | GATATCCAGATGACACAGACTACATCCTCCCTGTCTGCCTCTCTG | MAb52-25.1 moVL |
| | GGAGACAGAGTCACCATCGATTGCAGGGCGAGTCAGGACATCAAC | |
| | AATTATTTAAACTGGTATCAACAGAAACCAGATGGAACTGTTAAA | |
| | CTCCTGATCCACTACACATCAAGATTACACTCAGGAGTCCCATCA | |
| | AGGTTCAGAGGCAGTGGGTCTGGAACAGATTTTTCTCTCACCATT | |
| | ACCAACCTGGAACAAGAAGATGTTGCCACTTACTTTTGTCAACAG | |
| | GGTAATACGATGTGGACGTTCGGTGGAGGCACCAAGTTGGATATC | |
| | AAA | |
| 12 | DIQMTQTTSSLSASLGDRVTIDCRASQDINNYLNWYQQKPDGTVK | MAb52-25.1 moVL |
| | LLIHYTSRLHSGVPSRFRGSGSGTDFSLTITNLEQEDVATYFCQQ | |
| | GNTMWTFGGGTKLDIK | |
| 13 | GAAGTGAAACTGGTGGAGTCTGGGGGAGGCTTAGTGGAGCCTGGA | MAb52-25.1 moVH |
| | ACGTCTCTGAAACTCTCCTGTGCAGCCTCTGAATTCACTTTCAGT | |
| | AATTATGGAATGTCTTGGGTTCGCCAGACTTCAGACAAGAGGCTG | |
| | GAGTGGGTCGCATCCATTAGTAGTGGTAATGGGGTTTATTATTCA | |
| | GACAATGTAAAGGGCCGATTCACCATCTCCAGAGAGAATGCCAAG | |
| | AACACCCTGTATTTGCAAATGAGTAGTCTGAAGTCTGAAGACACG | |
| | GCCTTATATTACTGTGCAAGAGTTGATGGTAGTTATGTATATTTC | |
| | GATGTCTGGGGCTCAGGGACCACGGTCACCGTCTCCTCA | |
| 14 | EVKLVESGGGLVEPGTSLKLSCAASEFTFSNYGMSWVRQTSDKRL | MAb52-25.1 moVH |
| | EWVASISSGNGVYYSDNVKGRFTISRENAKNTLYLQMSSLKSEDT | |
| | ALYYCARVDGSYVYFDVWGSGTTVTVSS | |
| 15 | QDINNYLN | MAb52-25.1 CDR-L1 |
| 16 | YTSRLHS | MAb52-25.1 CDR-L2 |
| 17 | QQGNTMWT | MAb52-25.1 CDR-L3 |
| 18 | EFTFSNYGMS | MAb52-25.1 CDR-H1 |
| 19 | SISSGNGVYYSDNVKG | MAb52-25.1 CDR-H2 |
| 20 | VDGSYVYFDV | MAb52-25.1 CDR-H3 |
| 21 | DIQMTQSPSSLSASVGDRVTITCKASEDIYNRLAWYQQKPGKAPK | MAb52-29.1 HuVL1 |
| | LLLYGVSSLEAGVPSRFSGSGSGTDYTLTISSLQPEDFATYYCQQ | |
| | YWSSPYTFGQGTKLEIK | |
| 22 | DIQMTQSPSSLSASVGDRVTITCKASEDIYNRLAWYQQKPGKAPK | MAb52-29.1 HuVL2 |
| | LLISGVSSLEAGIPSRFSGSGSGTDYTLTISSLQPEDFAAYYCQQ | |
| | YWSSPYTFGQGTKLEIK | |
| 23 | DIQMTQSPSSLSASVGDRVTITCKASEDIYNRLAWYQQKPGNAPK | MAb52-29.1 HuVL3 |
| | LLISGVSSLEAGIPSRFSGSGSGTDYTLTISSLQPEDVAAYYCQQ | |
| | YWSSPYTFGQGTKLEIK | |
| 24 | DIQMTQSPSSLSASVGDRVTITCKASEDIYNRLAWYQQKPGNAPK | MAb52-29.1 HuVL4 |
| | LLISGVSSLEAGIPSRFSGSGSGKDYTLTISSLQPEDVAAYYCQQ | |
| | YWSSPYTFGQGTKLEIK | |
| 25 | EVKLVESGGGLVQPGGSLKLSCAASGFTFRTAFMSWIRQTPEKRL | MAb52-29.1 HuVH1 |
| | ELVAEISDSGGSTYFSDTVKGRFTISRDNAKNSLYLQMSSLKSED | |
| | TAMYYCARRGQLGLRGFFDSWGQGTTLTVSS | |
| 26 | EVQLVESGGGLVQPGGSLRLSCAASGFTFRTAFMSWVRQAPGKGL | MAb52-29.1 HuVH2 |
| | EWVSEISDSGGSTYFSDTVKGRFTISRDNAKNSLYLQMNSLRAED | |
| | TAVYYCARRGQLGLRGFFDSWGQGTLVTVSS | |
| 27 | EVQLVESGGGLVQPGGSLRLSCAASGFTFRTAFMSWIRQAPGKGL | MAb52-29.1 HuVH3 |
| | ELVAEISDSGGSTYFSDTVKGRFTISRDNAKNSLYLQMNSLRAED | |
| | TAVYYCARRGQLGLRGFFDSWGQGTLVTVSS | |
| 28 | EVQLVESGGGLVQPGGSLRLSCAASGFTFRTAFMSWIRQAPEKRL | MAb52-29.1 HuVH4 |
| | ELVAEISDSGGSTYFSDTVKGRFTISRDNAKNSLYLQMNSLRAED | |
| | TAMYYCARRGQLGLRGFFDSWGQGTLVTVSS | |
| 29 | TCCAGAACACGATGAGGCAGAC | ALCAM Forward |
| | | primer |
| 30 | GTAGACGACACCAGCAACAAGG | ALCAM Reverse |
| | | primer |
| 31 | GGCGACCTGGAAGTCCAACT | RPLP0 Forward |
| | | primer |
| 32 | CCATCAGCACCACAGCCTTC | RPLP0 Reverse |
| | | primer |
| 33 | CTGCTCACTGGATGGTGGGAAGATGG | Light chain reverse |
| | | primer |
| 34 | AGCTGGGAAGGTGTGCACAC | Heavy chain reverse |
| | | primer |
| 35 | CGGGGATTCTTTGACTCCTGGGGCCAAGGCACC | 29C102SF primer |
| 36 | GGTGCCTTGGCCCCAGGAGTCAAAGAATCCCCG | 29C102SR primer |
| 37 | RGQLGLRGFFDS | CDR-H3-C102S |
| 38 | AGCACTAGTGCCGCCACCATGAAGTTTCCTTCTCAAC | 29VL-F primer |
| 39 | GGGCGCCGCTACAGTCCGTTTTATTTCCAGTTTGG | 29VL-R primer |
| 40 | AGCTCTAGAGCCGCCACCATGAATTTCGGGCTCAG | 29VH-F primer |
| 41 | GGTGCTAGCTGAGGAGACTGTGAGAGTGGTGC | 29VH-R primer |
| 42 | AGAACTAGTGCCGCCACCATGTCCTCTGCTCAGTTCC | 25VL-F primer |
| 43 | CTGGGCGCCGCTACAGTCCGTTTGATATCCAACTTGG | 25VL-R primer |
| 44 | AGCTCTAGAGCCGCCACCATGAACTTCGGGCTCAGC | 25VH-F primer |
| 45 | GTGCTAGCTGAGGAGACGGTGACCGTGGTCCCTGAG | 25VH-R primer |
| 46 | DIQMTQSPSSLSASVGDRVTITCRASQGISNSLAWYQQKPGKAPK | human IGKV1-NL1*01 |
| | LLLYAASRLESGVPSRFSGSGSGTDYTLTISSLQPEDFATYYCQQ | |
| | YYSTP | |
| 47 | EVQLVESGGGLVQPGGSLRLSCAASGFTFSSYEMNWVRQAPGKGL | Human IGHV3-48*03 |
| | EWVSYISSSGSTIYYADSVKGRFTISRDNAKNSLYLQMNSLRAED | |
| | TAVYYCAR | |
| 48 | YTFGQGTKLEIK | human IGKJ2 |
| 49 | DSWGQGTLVTVSS | human IGHJ5 |

### REFERENCES

Bowen M A, Patel D D, Li X, Modrell B, Malacko A R, Wang W C, Marquardt H, Neubauer M, Pesando J M, Francke U, et al. (1995) Cloning, mapping, and characterization of activated leukocyte-cell adhesion molecule (ALCAM), a CD6 ligand. J Exp Med. 181:2213-20.
Cayrol R, Wosik K, Berard J L, Dodelet-Devillers A, Ifergan I, Kebir H, et al. (2007) Activated leukocyte cell adhesion molecule promotes leukocyte trafficking into the central nervous system. Nat Immunol 9:137-145.
Chitteti B R, Kobayashi M, Cheng Y, Zhang H, Poteat B A, Broxmeyer H E, Pelus L M, Hanenberg H, Zollman A, Kamocka M M, Carlesso N, Cardoso A A, Kacena M A,Srour E F (2014) CD166 regulates human and murine hematopoietic stem cells and the hematopoietic niche. Blood. 124:519-29.
Darvishi B, Boroumandieh S, Majidzadeh-A K, Salehi M, Jafari F, Farahmand L (2020) The role of activated leukocyte cell adhesion molecule (ALCAM) in cancer progression, invasion, metastasis and recurrence: A novel cancer stem cell marker and tumor-specific prognostic marker. Exp Mol Pathol. 2020.104443
Ferragut F, Vachetta V S, Troncoso M F, Rabinovich G A, Elola M T (2021) ALCAM/CD166: A pleiotropic mediator of cell adhesion, stemness and cancer progression. Cytokine Growth Factor Rev. 61:27-37.
Fujiwara K, Ohuchida K, Sada K, Horioka K, Ulrich C D, Shindo K, Ohtsuka T, Takahata S, Mizumoto K, Oda Y, Tanaka M (2014) CD166/ALCAM expression is characteristic of tumorigenicity and invasive and migratory activities of pancreatic cancer cells. PLOS One. 9: e107247.
Gogia P, Ashraf H, Bhasin S, Xu Y (2023) Antibody-Drug Conjugates: A Review of Approved Drugs and Their Clinical Level of Evidence. Cancers (Basel) 15:3886.
Gurrea-Rubio M and Fox D A (2022) The dual role of CD6 as a therapeutic target in cancer and autoimmune disease. Front Med (Lausanne). 9:1026521.
Hassan Nj, Barclay A N, Brown M H (2004) Frontline: Optimal T cell activation requires the engagement of CD6 and CD166. Eur J Immunol. 34:930-40.
Hong X, Michalski C W, Kong B, Zhang W, Raggi M C, Sauliunaite D, Oliveira T D, Friess H, Kleeff J (2010) ALCAM Is Associated With Chemoresistance and Tumor Cell Adhesion in Pancreatic Cancer. J Surg Oncol. 101:564-9.
Ihnen M, Müller V, Wirtz R M, Schröder C, Krenkel S, Witzel I, Lisboa B W, Jänicke F, Milde-Langosch K (2008) Predictive impact of activated leukocyte cell adhesion molecule (ALCAM/CD166) in breast cancer. Breast Cancer Res Treat. 112:419-27.
Lee, N K, Su Y, Bidlingmaier S, Liu B (2019) Manipulation of cell-type selective antibody internalization by a guide-effector bispecific design. Mol. Cancer Ther. 18:1092-103.
Liu K, Li M, Li Y, Li Y, Chen Z, Tang Y, Yang M, Deng G, Liu H (2024) A review of the clinical efficacy of FDA-approved antibody-drug conjugates in human cancers. Mol Cancer. 23:62. Junutula J, Raab H, Clark S, et al. (2008) Site-specific conjugation of a cytotoxic drug to an antibody improves the therapeutic index. Nat Biotechnol. 26:925-932.
Roth A, Drummond D C, Conrad F, Hayes M E, Kirpotin D B, Benz C C, Marks J D, Liu B (2007) Anti-CD166 single chain antibody-mediated intracellular delivery of liposomal drugs to prostate cancer cells. Mol. Cancer Ther. 6:2737-2746.
van Kempen L C, Nelissen J M, Degen W G, et al. (2001) Molecular basis for the homophilic activated leukocyte cell adhesion molecule (ALCAM)-ALCAM interaction. J Biol Chem 276:25783-25790.
von Lersner A, Droesen L, Zijlstra A (2019) Modulation of cell adhesion and migration through regulation of the immunoglobulin superfamily member ALCAM/CD166. Clin Exp Metastasis. 36:87-95.
Wang Y, Yu W, Zhu J, et al. (2019) Anti-CD166/4-1B B chimeric antigen receptor T cell therapy for the treatment of osteosarcoma. J Exp Clin Cancer Res. 38:168.
Wiiger M T, Gehrken H B, Fodstad O, Maelandsmo G M, Andersson Y (2010) A novel human recombinant single-chain antibody targeting CD166/ALCAM inhibits cancer cell invasion in vitro and in vivo tumour growth. Cancer Immunol Immunother 59:1665-74.
Yang Y, Sanders A J, Dou Q P, Jiang D G, Li A X, Jiang W G (2021) The Clinical and Theranostic Values of Activated Leukocyte Cell Adhesion Molecule (ALCAM)/CD166 in Human Solid Cancers. Cancers (Basel). 13:

What is claimed is:

1. A binding agent of human CD166 including a light chain variable region wherein CDR1 to CDR3 have sequences at least 70% identical to, respectively, SEQ ID NO: 5, SEQ ID NO: 6, and SEQ ID NO: 7.

2. The binding agent of claim 1, wherein CDR1 to CDR3 of the light chain variable region have sequences identical to, respectively, SEQ ID NO: 5, SEQ ID NO: 6, and SEQ ID NO: 7.

3. A binding agent having a light chain variable region which is at least 70% identical to SEQ ID NO: 2.

4. A binding agent including a heavy chain variable region wherein CDR1 to CDR3 have sequences at least 70% identical to, respectively, SEQ ID NO: 8, SEQ ID NO: 9, and SEQ ID NO: 10.

5. The binding agent of claim 4, wherein CDR1 to CDR3 of the heavy chain variable region have sequences identical to, respectively, SEQ ID NO: 8, SEQ ID NO: 9, and SEQ ID NO: 37.

6. A binding agent having a heavy chain variable region which is at least 70% identical to SEQ ID NO: 4.

7. An isolated DNA or RNA molecule encoding the light chain variable region of a binding agent of claim 2.

8. An isolated DNA molecule of claim 7 having the nucleotide sequence of SEQ ID NO: 1.

9. An isolated DNA or RNA molecule encoding the heavy chain variable region of a binding agent of claim 5.

10. An isolated DNA molecule of claim 9 having the nucleotide sequence of SEQ ID NO: 3.

11. The binding agent of claim 1 or 4 wherein the binding agent is a monoclonal antibody.

12. The binding agent of claim 11 wherein the monoclonal antibody is a murine, human, humanized, chimeric, bispecific, or multi-specific antibody.

13. The binding agent of claim 1 wherein the binding agent is a diabody, a single domain antigen binding (SDAB) molecule, or a VL domain.

14. The binding agent of claim 4 wherein the binding agent is a diabody, a single domain antigen binding (SDAB) molecule, a VH domain, or a VHH domain.

15. The binding agent of claim 11 wherein the monoclonal antibody has an IgGl heavy chain and a κ light chain.

16. The binding agent of claim 1 or 4 wherein the binding agent is a Fab, Fab′, F(ab′)2, rIgG, Fv, or Fd fragment.

17. The binding agent of claim 1 or 4 wherein the binding agent is a scFv or a sc (Fv)₂.

18. The binding agent of claim 11 wherein the monoclonal antibody is class: IgD, IgE, IgG, IgA, or IgM, or a sub-class of one of said classes.

19. The monoclonal antibody of claim 18 wherein the sub-class is IgGI, IgG2, IgG3, IgG4, IgAQ1 or IgA2.

20. The monoclonal antibody of claim 18 having a κ light chain.

21. A vector comprising the isolated DNA molecules of claim 8 or 10.

22. The binding agent of claim 1 or 4 conjugated with an anti-tumor agent.

23. The binding agent of claim 22 wherein the anti-tumor agent is monomethyl auristatin E.

23. The binding agent of claim 22 wherein the conjugation is with a linker.

24. The binding agent of claim 23 wherein the linker is cleavable or non-cleavable.

25. The binding agent of claim 24 wherein the linker is a lysosomal-cleavable dipeptide linker.

26. The binding agent of claim 25 wherein the lysosomal-cleavable dipeptide linker includes a para-aminobenzyloxycarbonyl (PABC) spacer and a thiol-reactive maleimidocaproyl group that specifically reacts with a free thiol group in cysteines, as well as anti-tubulin toxin monomethyl auristatin E.

27. A binding agent of human CD166 including a light chain variable region wherein CDR1 to CDR3 have sequences at least 70% identical to, respectively, SEQ ID NO: 15, SEQ ID NO: 16, and SEQ ID NO: 17.

28. A binding agent of human CD166 including a heavy chain variable region wherein CDR1 to CDR3 have sequences at least 70% identical to, respectively, SEQ ID NO: 18, SEQ ID NO: 19, and SEQ ID NO: 20.
